SEQUENCE LISTING (58 sequences)

SEQ ID NO: 1 - protein, 31 aa, Homo sapiens
HAEGTFTSDVSSYLEGQAAKEFIAWLVKGRG

SEQ ID NO: 2 - protein, 31 aa, synthetic construct
  SITE 2: note = Aib
HXEGTFTSDVSSYLEGQAAKEFIAWLVRGRG

SEQ ID NO: 3 - protein, 31 aa, synthetic construct; identical to SEQ ID NO: 2
  SITE 2: note = Aib
  MOD_RES 31: note = C-terminus is amidated

SEQ ID NO: 4 - protein, 39 aa, synthetic construct
HGEGTFTSDLSKQMEEEAVRLFIEWLKNGGPSSGAPPPS

SEQ ID NO: 5 - protein, 39 aa, synthetic construct; identical to SEQ ID NO: 4
  MOD_RES 39: note = C-terminus is amidated

SEQ ID NO: 6 - protein, 44 aa, synthetic construct
  MOD_RES 44: note = C-terminus is amidated
HGEGTFTSDLSKQMEEEAVRLFIEWLKNGGPSSGAPPSKKKKKK

SEQ ID NO: 7 - protein, 44 aa, synthetic construct; identical to SEQ ID NO: 6

SEQ ID NO: 8 - protein, 39 aa, synthetic construct
  SITE 2: note = D-Ala
  SITE 14: note = Nle
HAEGTFTSDLSKQXEEEAVRLFIEWLKQGGPSSGAPPPC

SEQ ID NO: 9 - protein, 39 aa, synthetic construct; identical to SEQ ID NO: 8
  SITE 2: note = D-alanine
  SITE 14: note = Nle
  MOD_RES 39: note = C-terminus is amidated

SEQ ID NO: 10 - protein, 39 aa, synthetic construct; identical to SEQ ID NO: 8
  SITE 2: note = D-alanine
  SITE 14: note = Nle
  SITE 39: note = sulfur of cysteine is PEGylated

SEQ ID NO: 11 - protein, 39 aa, synthetic construct; identical to SEQ ID NO: 8
  SITE 2: note = D-alanine
  SITE 14: note = Nle
  SITE 39: note = sulfur of cysteine is PEGylated
  MOD_RES 39: note = C-terminus is amidated

SEQ ID NO: 12 - protein, 31 aa, synthetic construct
HAEGTFTSDVSSYLEGQAAKEFIAWLVRGRG

SEQ ID NO: 13 - protein, 31 aa, synthetic construct; identical to SEQ ID NO: 12
  MOD_RES 31: note = C-terminus is amidated

SEQ ID NO: 14 - protein, 31 aa, synthetic construct
HVEGTFTSDVSSYLEEQAAREFIKWLVRGRG

SEQ ID NO: 15 - protein, 31 aa, synthetic construct; identical to SEQ ID NO: 14
  MOD_RES 31: note = C-terminus is amidated

SEQ ID NO: 16 - protein, 31 aa, synthetic construct
HGEGTFTSDVSSYLEGQAAKEFIAWLVRGRG

SEQ ID NO: 17 - protein, 31 aa, synthetic construct; identical to SEQ ID NO: 16
  MOD_RES 31: note = C-terminus is amidated

SEQ ID NO: 18 - protein, 32 aa, synthetic construct
  SITE 2: note = Aib
HXEGTFTSDVSSYLEGQAAKEFIAWLVRGRGL

SEQ ID NO: 19 - protein, 32 aa, synthetic construct; identical to SEQ ID NO: 18
  SITE 2: note = Aib
  MOD_RES 32: note = C-terminus is amidated

SEQ ID NO: 20 - protein, 39 aa, synthetic construct
  SITE 2: note = Aib
  SITE 13: note = Aib
  MOD_RES 39: note = C-terminus is amidated
YXEGTFTSDYSIXLDKIAQKAFVQWLIAGGPSSGAPPPS

SEQ ID NO: 21 - protein, 39 aa, synthetic construct; identical to SEQ ID NO: 20
  SITE 2: note = Aib
  SITE 13: note = Aib

SEQ ID NO: 22 - protein, 30 aa, synthetic construct
HSQGTFTSDKSEYLDSERARDFVAWLEAGG

SEQ ID NO: 23 - protein, 30 aa, synthetic construct; identical to SEQ ID NO: 22
  MOD_RES 30: note = C-terminus is amidated

SEQ ID NO: 24 - protein, 29 aa, synthetic construct
  SITE 2: note = 1-amino-cyclobutanecarboxylic acid (Ac4c)
  MOD_RES 29: note = C-terminus is amidated
HXQGTFTSDYSKYLDERAAKDFIKWLESA

SEQ ID NO: 25 - protein, 29 aa, synthetic construct
  SITE 2: note = 1-amino-cyclobutanecarboxylic acid (Ac4c)
HXQGTFTSDYSKYLDERAAKDFIKWLESA

SEQ ID NO: 26 - protein, 29 aa, synthetic construct
  SITE 2: note = Aib
  SITE 16: note = E at position 16 and K at position 20 are connected via a lactam bridge
  SITE 20: note = E at position 16 and K at position 20 are connected via a lactam bridge
HXQGTFTSDYSKYLDEKAAKEFIQWLLQT

SEQ ID NO: 27 - protein, 29 aa, synthetic construct
  SITE 2: note = Aib
  SITE 16: note = E at position20 and K at position 20 are connected via a lactam bridge
  SITE 20: note = E at position20 and K at position 20 are connected via a lactam bridge
  MOD_RES 29: note = C-terminus is amidated
HXQGTFTSDYSKYLDEKAAKEFIQWLLQT

SEQ ID NO: 28 - protein, 34 aa, synthetic construct
  SITE 2: note = Aib
  MOD_RES 34: note = C-terminus is amidated
HXQGTFTSDYSKYLDEKKAKEFVEWLLEGGPSSG

SEQ ID NO: 29 - protein, 34 aa, synthetic construct; identical to SEQ ID NO: 28
  SITE 2: note = Aib

SEQ ID NO: 30 - protein, 33 aa, synthetic construct
  SITE 2: note = Aib
HXEGSFTSELATILDKQAARDFIAWLIQHKITD

SEQ ID NO: 31 - protein, 33 aa, synthetic construct; identical to SEQ ID NO: 30
  SITE 2: note = Aib
  MOD_RES 33: note = C-terminus is amidated

SEQ ID NO: 32 - protein, 39 aa, synthetic construct
  SITE 2: note = Aib
  SITE 13: note = alpha-methyl-leucine (alphaMeL)
  SITE 20: note = Aib
  MOD_RES 39: note = C-terminus is amidated
YXQGTFTSDYSIXLDKKAQXAFIEYLLEGGPSSGAPPPS

SEQ ID NO: 33 - protein, 39 aa, synthetic construct; identical to SEQ ID NO: 32
  SITE 2: note = Aib
  SITE 13: note = alpha-methyl-leucine (alphaMeL)
  SITE 20: note = Aib

SEQ ID NO: 34 - protein, 18 aa, synthetic construct
WWEQDRDWDFDVFGGGTP

SEQ ID NO: 35 - protein, 11 aa, synthetic construct
  DISULFID 3..9: note = cysteines at position 3 and 9 are connected via a disulfide bridge
DICLPRWGCLW

SEQ ID NO: 36 - protein, 18 aa, synthetic construct
  DISULFID 7..13: note = cysteines at position 7 and 13 are connected via a disulfide bridge
RLIEDICLPRWGCLWEDD

SEQ ID NO: 37 - protein, 46 aa, synthetic construct
LAEAKVLANRELDKYGVSDFYKRLINKAKTVEGVEALKLHILAALP

SEQ ID NO: 38 - protein, 46 aa, synthetic construct
IAEAKEAANAELDSYGVSDFYKRLIDKAKTVEGVEALKDAILAALP

SEQ ID NO: 39 - protein, 8 aa, synthetic construct
  SITE 1: note = fluorescein-(AEEA), wherein AEEA is 2-(2-(2-aminoethoxy)acetyl
  SITE 5: note = K(palmitate)
XEYEXEYE

SEQ ID NO: 40 - protein, 31 aa, synthetic construct; identical to SEQ ID NO: 2
  SITE 2: note = Aib
  LIPID 20: note = ([2-(2-amino-ethoxy)-ethoxy]-acetyl)2-(gamma-Glu)-C O-(CH2)16-CO2H

SEQ ID NO: 41 - protein, 31 aa, synthetic construct; identical to SEQ ID NO: 12
  LIPID 20: note = palmitoyl-Gln-OH

SEQ ID NO: 42 - protein, 31 aa, synthetic construct; identical to SEQ ID NO: 14
  LIPID 24: note = ([2-(2-amino-ethoxy)-ethoxy]-acetyl)2-(gamma-Glu)-C O-(CH2)16-CO2H

SEQ ID NO: 43 - protein, 31 aa, synthetic construct; identical to SEQ ID NO: 16
  LIPID 20: note = ([2-(2-amino-ethoxy)-ethoxy]-acetyl)2-(gamma-Glu)-C O-(CH2)20-CO2H

SEQ ID NO: 44 - protein, 32 aa, synthetic construct; identical to SEQ ID NO: 18
  SITE 2: note = Aib
  LIPID 20: note = AEEA-Aib-gammaGlu-ODDA

SEQ ID NO: 45 - protein, 39 aa, synthetic construct; identical to SEQ ID NO: 20
  SITE 2: note = Aib
  SITE 13: note = Aib
  LIPID 20: note = ([2-(2-amino-ethoxy)-ethoxy]-acetyl)2-(gammaGlu)1-C O-(CH2)18-CO2H
  MOD_RES 39: note = C-terminus is amidated

SEQ ID NO: 46 - protein, 30 aa, synthetic construct; identical to SEQ ID NO: 22
  LIPID 10: note = gamma-Glu-palmitoyl

SEQ ID NO: 47 - protein, 29 aa, synthetic construct
  SITE 2: note = 1-amino-cyclobutanecarboxylic acid (Ac4c)
  LIPID 24: note = [l7-carboxy-heptadecanoyl]-isoGlu-GSGSGG
  MOD_RES 29: note = C-terminus is amidated
HXQGTFTSDYSKYLDERAAKDFIKWLESA

SEQ ID NO: 48 - protein, 29 aa, synthetic construct
  SITE 2: note = Aib
  SITE 16: note = E at position 16 and the K at position 20 are connected via a lactam bridge
  SITE 20: note = E at position 16 and the K at position 20 are connected via a lactam bridge
  LIPID 17: note = glucuronic acid C-18
  MOD_RES 29: note = C-terminus is amidated
HXQGTFTSDYSKYLDEKAAKEFIQWLLQT

SEQ ID NO: 49 - protein, 34 aa, synthetic construct; identical to SEQ ID NO: 28
  SITE 2: note = Aib
  LIPID 20: note = ([2-(2-amino-ethoxy)-ethoxy]-acetyl)2-(gamma-Glu)-C O-(CH2)18-CO2H
  MOD_RES 34: note = C-terminus is amidated

SEQ ID NO: 50 - protein, 33 aa, synthetic construct; identical to SEQ ID NO: 30
  SITE 2: note = Aib
  LIPID 16: note = [17-carboxy-heptadecanoyl]-isoGlu

SEQ ID NO: 51 - protein, 39 aa, synthetic construct; identical to SEQ ID NO: 32
  SITE 2: note = Aib
  SITE 13: note = alpha-methyl-leucine (alphaMeL)
  SITE 20: note = Aib
  LIPID 17: note = (2-[2-(2-amino-ethoxy)-ethoxy]-acetyl)-(gammaGlu)-C O-(CH2)18-CO2H
  MOD_RES 39: note = C-terminus is amidated

SEQ ID NO: 52 - protein, 35 aa, synthetic construct
  MOD_RES 1: note = D-Lysine, wherein the side chain is acylated with COC16H32CO2H via a gamma Glu-(MINIPEG)2-gamma Glu spacer, wherein MINIPEG is COCH2OCH2CH2OCH2CH2NH
  SITE 2: note = n-methyl glycine (sarcosine)
  MOD_RES 35: note = side chain of lysine is acylated with COC16H32CO2H via a gamma Glu-(MINIPEG)2-gamma Glu spacer, wherein MINIPEG is COCH2OCH2CH2OCH2CH2NH
KXSVSEIQLMHNLGKHLNSMERVEWLRKKLQDVHK

SEQ ID NO: 53 - protein, 35 aa, synthetic construct; identical to SEQ ID NO: 52
  MOD_RES 1: note = D-Lysine, wherein the side chain is acylated with COC16H32CO2H via a gamma Glu-(MINIPEG)2-gamma Glu spacer, wherein (miniPEG)2 is COCH2OCH2CH2OCH2CH2NH
  SITE 2: note = n-methyl glycine (sarcosine)
  MOD_RES 35: note = side chain of lysine is acylated with COC16H32CO2H via a gamma Glu-(MINIPEG)2-gamma Glu spacer, wherein (miniPEG)2 is COCH2OCH2CH2OCH2CH2NH

SEQ ID NO: 54 - protein, 40 aa, synthetic construct
  SITE 2: note = AiB
HXEGTFTSDVSSYLEEEAAKEFIAWLVRGGPSSGAPPPSK

SEQ ID NO: 55 - protein, 31 aa, synthetic construct
  SITE 2: note = Aib
HXEGTFTSDVSSYLEEQAAKEFIAWLVRGGG

SEQ ID NO: 56 - protein, 71 aa, synthetic construct
  SITE 2: note = Aib
HXEGTFTSDVSSYLEEQAAKEFIAWLVRGGGGAQPGAQPGAQPGAQPGAQPGAQPGAQPGAQPGAQPGQKP

SEQ ID NO: 57 - protein, 39 aa, synthetic construct; identical to SEQ ID NO: 20
  SITE 13: note = AiB
  SITE 2: note = 2; note = Aib

SEQ ID NO: 58 - protein, 191 aa, Homo sapiens [sequence omitted]

(19) United States
(12) Patent Application Publication
(10) Pub. No.: US 2026/0069702 A1
(43) Pub. Date: Mar. 12, 2026

(54) Drug Compounds Comprising Albumin-Binding Moieties

(71) Applicant: Ascendis Pharma A/S, Hellerup (DK)

(72) Inventors: Kennett SPROGØE, Hellerup (DK); Nicola BISEK, Heidelberg (DE); Samuel WEISBROD, Heidelberg (DE)

(21) Appl. No.: 19/257,318

(22) Filed: Jul. 1, 2025

Related U.S. Application Data
(63) Continuation of application No. PCT/EP2024/055725, filed on Mar. 5, 2024.

(30) Foreign Application Priority Data
Mar. 6, 2023 (EP) 23160259.0
Oct. 16, 2023 (EP) 23203738.2

(51) Int. Cl. A61K 47/64; A61K 9/00

(52) U.S. Cl. CPC A61K 47/64; A61K 9/0019

(57) ABSTRACT

The present invention relates to compounds or a pharmaceutically acceptable salt thereof of formula (Ia) or (Ib), wherein each -D- is independently a drug moiety; each -AB¹ and -AB² is independently an albumin-binding moiety; each -L¹- is independently a linker moiety covalently and reversibly connected to -D-; each -L²- is independently a single chemical bond or is absent; and x and y are an integer; to pharmaceutical compositions comprising at least one such compound and to their uses.

### CROSS REFERENCE TO RELATED APPLICATIONS

This application is a continuation of PCT/EP2024/055725 filed Mar. 5, 2024, incorporated by reference in its entirety for all purposes, which claims priority to EP Patent Application No. 23160259.0 filed Mar. 6, 2023, and EP Patent Application No. 23203738.2 filed Oct. 16, 2023.

### REFERENCE TO AN ELECTRONIC SEQUENCE LISTING

The contents of the electronic sequence listing (633695SEQLST.xml: size 88,000 bytes, created Jun. 27, 2025) are herein incorporated by reference in their entirety.

The present invention relates to compounds or a pharmaceutically acceptable salt thereof of formula (Ia) or (Ib), wherein each -D- is independently a drug moiety; each -AB¹ and -AB² is independently an albumin-binding moiety; each -L¹- is independently a linker moiety covalently and reversibly connected to -D-; each -L²- is independently a single chemical bond or is absent; and x and y are an integer; to pharmaceutical compositions comprising at least one such compound and to their uses.

Whereas some classes of therapeutic proteins, including antibodies, have long inherent half-lives, many other molecules, particularly protein or peptide hormones are often susceptible to enzymatic degradation, renal clearance and/or rapid receptor-mediated clearance, which leads to a short plasma elimination half-life. Furthermore, due to the challenges associated with oral delivery, most peptide- or protein-based drugs require parenteral administration, which combined with a short half-life necessitates frequent injections, resulting in discomfort and inconvenience for the patient. This may negatively impact adherence and, ultimately, limit treatment efficacy and outcomes.

Injecting lipidated peptides has been demonstrated to have slower absorption from the subcutaneous tissue compared to the non-derivatized peptide as well as longer circulatory half-life due to reversible binding to albumin. The half-life of endogenous albumin in the circulation is approximately 3 weeks. This long half-life can be attributed both to its large molecular size, which minimizes renal clearance, as well as to recycling via the neonatal Fc receptor (FcRn).

In the circulation, the large molecular size protects the bound peptide from renal clearance and reduces the rate of distribution to the extravascular compartment.

The slower absorption is thought to be due to a reduced rate of diffusion in the tissue by a mechanism involving interaction of the fatty acid side chain with cell membranes and/or proteins such as albumin present at the injection site. The rate of diffusion in the tissue following injection and the passage over the capillary wall would expectedly be reduced due to the large molecular size of the albumin-peptide complex. Molecules with a molecular size greater than approximately 16 kDa are preferentially alternatively be absorbed via a lymphatic route compared to direct absorption into blood across the capillary wall.

Thus, slower absorption and reversible albumin binding has enabled extension of dosing intervals for drugs, such as peptide drugs, to up to a week. However, an even further extension of the dosing intervals would be desirable.

It is an object of the present invention to overcome the shortcomings of current therapies at least partially.

This object is achieved with a compound or a pharmaceutically acceptable salt thereof of formula (Ia) or (Ib)

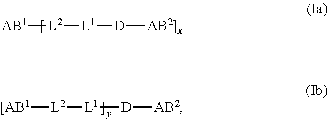

wherein
each -D- is independently a drug moiety;
each -AB¹ and -AB² is independently an albumin-binding moiety;
each -L¹- is independently a linker moiety covalently and reversibly connected to -D-;
each -L²- is independently a spacer or is absent;
x is an integer selected from the group consisting of 1, 2, 3, 4, 5, 6, 7, 8, 9, 10, 11, 12, 13, 14, 15, 16, 17, 18, 19, 20, 21, 22, 23, 24 and 25; and
y is an integer selected from the group consisting of 2, 3, 4 and 5.

The compounds of formula (Ia) and (Ib) may also be referred to as “prodrugs”. They release a drug moiety-AB² conjugate, which may also be written as H-D-AB², wherein H— is hydrogen.

Applicant discovered that the compounds of the present invention have an extended half-life that allow for a reduced dosing frequency.

This results in compounds exhibiting a further increased albumin binding and slowed absorption of the macromolecular drug-albumin complex. Such compounds also demonstrate a reduced absorption from the site of administration, such as from subcutaneous tissue, compared to administration of the corresponding unconjugated drugs. Consequently, such compounds have a prolonged half-life compared to the corresponding unconjugated drugs.

Within the present invention the terms are used having the meaning as follows.

As used herein, the term “GLP-1 receptor agonist” refers to agonists of the GLP-1 receptor (GLP1R) and optionally in addition agonists of one or more other receptors, such as for example a receptor for gastric inhibitory polypeptide (GIPR), a receptor for glucagon (GCGR), a receptor for amylin, a receptor for peptide YY (PYYR) or a receptor for glucagon-like peptide-2 (GLP2R). An “agonist” of a receptor, such as an agonist of the GLP-1 receptor, is a chemical compound that activates such receptor to produce a biological response. If a GLP-1 receptor agonist is in addition to being a GLP1R agonist also an agonist of one receptor other than GLP1R, such as an agonist of GIPR, GCGR, an amylin receptor, a PYYR or GLP2R, such GLP-1 receptor agonist is also referred to as being a “dual GLP-1 receptor agonist” or short “dual agonist”. Likewise, if a GLP-1 receptor agonist is in addition to being a GLP1R agonist also an agonist of two other receptors, which may be selected from the group consisting of GIPR, GCGR, an amylin receptor, a PYYR or GLP2R, such GLP-1 receptor agonist is also referred to as being a “triple GLP-1 receptor agonist” or short “triple agonist”.

As used herein, the term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties. The cyclic peptides may be mono-, bi-, tri- or tetracyclic peptides. The term “peptide” also includes lasso peptides.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “small molecule drug” refers to drugs that are organic compounds with a molecular weight of less than 1000 Da, such as less than 900 Da or less than 800 Da. It is understood that nucleobase-based drug moieties, such as adenine or guanine analogues, may also be a type of small molecule drugs.

As used herein, the term “medium molecule drug” or “medium size molecule drug” refer to drugs that are organic compounds which are not peptides and which are not proteins and have a molecular weight ranging from and including 1 kDa to 7.5 kDa.

As used herein, the terms “oligonucleotide” refers to double- or single-stranded RNA and DNA with preferably 2 to 1000 nucleotides and any modifications thereof. Modifications include, for example, those which provide other chemical groups that incorporate additional charge, polarizability, hydrogen bonding, electrostatic interaction, and fluxionality to the nucleic acid ligand bases or to the nucleic acid ligand as a whole. Such modifications include for example, to 2′-position sugar modifications, 5-position pyrimidine modifications, 8-position purine modifications, modifications at exocyclic amines, substitution of 4-thiouridines, substitution of 5-bromo or 5-iodo-uracil; backbone modifications, methylations, unusual base-pairing combinations such as the isobases isocytidine and isoguanosine. Modifications can also include 3′ and 5′ modifications such as capping and change of stereochemistry. The term also includes aptamers.

As used herein, the term “peptide nucleic acids” refers to organic polymers having a peptidic backbone, i.e., a backbone in which the monomers are connected to each other through peptide linkages, to which nucleobases such as adenine, cytosine, guanine, thymine and uracil, are attached. In certain embodiments, the peptide backbone comprises N-(2-aminoethyl)-glycine.

As used herein, the term “random coil” relates to any conformation of a polymeric molecule, including proteins, in which the individual monomeric elements that form said polymeric structure are essentially randomly oriented towards the adjacent monomeric elements while still being chemically bound to said adjacent monomeric elements. In particular, a polypeptide or protein having random coil conformation substantially lacks a defined secondary and tertiary structure. The nature of polypeptide random coils and their methods of experimental identification are known to the person skilled in the art. In particular, the lack of secondary and tertiary structure of a protein may be determined by circular dichroism (CD) measurements. CD spectroscopy represents a light absorption spectroscopy method in which the difference in absorbance of right- and left-circularly polarized light by a substance is measured. The secondary structure of a protein can be determined by CD spectroscopy using far-ultraviolet spectra with a wavelength between approximately 190 and 250 nm. At these wavelengths the different secondary structures commonly found in conformations each give rise to a characteristic shape and magnitude of the CD spectrum. Accordingly, by using CD spectrometry the skilled artisan is readily capable of determining whether an amino acid polymer adopts random coil conformation at physiological conditions.

When determining whether a peptide or protein adopts random coil conformation under experimental conditions using the methods as described herein, the biophysical parameters such as temperature, pH, osmolarity and protein content may be different to the physiological conditions normally found in vivo. Temperatures between 1° C. and 42° C. or preferably 4° C. to 25° C. may be considered useful to test and/or verify the biophysical properties and biological activity of a peptide or protein under physiological conditions in vitro.

Several buffers, in particular in experimental settings (for example in the determination of protein structures, in particular in circular dichroism (CD) measurements and other methods that allow the person skilled in the art to determine the structural properties of a protein/polypeptide or peptide stretch) or in buffers, solvents and/or excipients for pharmaceutical compositions, are considered to represent “physiological solutions” or “physiological conditions” in vitro. Examples of such buffers are, e.g. phosphate-buffered saline (PBS: 115 mM NaCl, 4 mM KH₂PO₄, 16 mM Na₂HPO₄ pH 7.4), Tris buffers, acetate buffers, citrate buffers or similar buffers such as those used in the appended examples. Generally, the pH of a buffer representing physiological conditions should lie in a range from 6.5 to 8.5, preferably in a range from 7.0 to 8.0, most preferably in a range from 7.2 to 7.7 and the osmolarity should lie in a range from 10 to 1000 mmol/kg H₂O, more preferably in a range from 50 to 500 mmol/kg H₂O and most preferably in a range from 200 to 350 mmol/kg H₂O. Optionally, the protein content of a buffer representing physiological conditions may lie in a range from 0 to 100 g/l, neglecting the protein with biological activity itself, whereby typical stabilizing proteins may be used, for example human or bovine serum albumin.

Other established biophysical methods for determining random coil conformation include nuclear magnetic resonance (NMR) spectroscopy, absorption spectrometry, infrared and Raman spectroscopy, measurement of the hydrodynamic volume via size exclusion chromatography, analytical ultracentrifugation and dynamic/static light scattering as well as measurements of the frictional coefficient or intrinsic viscosity.

As used herein the term “physiological conditions” refers to aqueous buffer at pH 7.4, 37° C.

As used herein the term “pharmaceutical composition” refers to a composition containing one or more active ingredients, such as for example at least one compound of the present invention, and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, a pharmaceutical composition of the present invention encompasses any composition made by admixing one or more compound of the present invention and one or more pharmaceutically acceptable excipient.

As used herein, the term “excipient” refers to a diluent, adjuvant, or vehicle with which the therapeutic, such as a drug or prodrug, is administered. Such pharmaceutical excipient may be sterile liquids, such as water and oils, including those of petroleum, animal, vegetable or synthetic origin, including but not limited to peanut oil, soybean oil, mineral oil, sesame oil and the like. Water is an example for an excipient when the pharmaceutical composition is administered orally. Saline and aqueous dextrose are examples of excipients when the pharmaceutical composition is administered intravenously. Saline solutions and aqueous dextrose and glycerol solutions are in certain embodiments employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, or sustained-release formulations. The pharmaceutical composition may be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

As used herein the term “liquid composition” refers to a mixture comprising a water-soluble compound and one or more solvents, such as water.

The term “suspension composition” relates to a mixture comprising at least one water-insoluble compound and one or more solvents, such as water.

As used herein, the term “dry composition” means that a pharmaceutical composition is provided in a dry form. Suitable methods for drying are spray-drying and lyophilization, i.e., freeze-drying. Such dry composition has a residual water content of a maximum of 10%, such as less than 5% or less than 2%, determined according to Karl Fischer. In certain embodiments such dry pharmaceutical composition is dried by lyophilization.

The term “drug” as used herein refers to a substance used in the treatment, cure, prevention, or diagnosis of a disease or used to otherwise enhance physical or mental well-being. If a drug is conjugated to another moiety, the moiety of the resulting product that originated from the drug is referred to as “drug moiety”.

As used herein the term “prodrug” refers to a covalent conjugate in which a drug moiety is reversibly and covalently connected to a specialized protective group through a reversible linker moiety, also referred to as “reversible prodrug linker moiety” or “reversible linker moiety”, which is conjugated through a reversible linkage to the drug moiety and wherein the specialized protective group alters or eliminates undesirable properties in the parent molecule. This also includes the enhancement of desirable properties in the drug and the suppression of undesirable properties. The specialized non-toxic protective group is referred to as “carrier”. A prodrug releases the reversibly and covalently bound drug moiety in the form of its corresponding drug. In other words, a prodrug is a conjugate comprising a drug moiety which is covalently and reversibly conjugated to a carrier moiety via a reversible linker moiety, which covalent and reversible conjugation of the carrier to the reversible linker moiety is either directly or through a spacer. Such conjugate releases the formerly conjugated drug moiety in the form of a free unmodified drug.

A “reversible linkage” is a linkage that is degradable, i.e., cleavable, in the absence of enzymes under physiological conditions (aqueous buffer at pH 7.4, 37° C.) with a half-life ranging from 1 hour to three months. A “stable linkage” is a linkage having a half-life under physiological conditions (aqueous buffer at pH 7.4, 37° C.) in the absence of enzymes of more than three months.

As used herein, the terms “traceless prodrug linker” or “traceless linker” means a reversible prodrug linker, i.e., a linker moiety reversibly and covalently connecting a drug moiety with a carrier, which upon cleavage releases the drug in its free form. As used herein, the term “free form” of a drug or “free drug” means the drug in its unmodified, pharmacologically active form.

As used herein, the term “reagent” means a chemical compound which comprises at least one functional group for reaction with the functional group of another chemical compound or drug. It is understood that a drug comprising a functional group, such as a primary or secondary amine or hydroxyl functional group, is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “—” indicates attachment to another moiety. Accordingly, a drug moiety is released from a prodrug as a drug.

If a chemical structure of a group of atoms is provided, which group of atoms is attached to at least one other moiety, said chemical structure may be attached to the at least one other moiety in either orientation, unless explicitly stated otherwise. For example, a moiety “—C(O)N(R¹)—” may be attached to two moieties either as “—C(O)N(R¹)—” or as “—N(R¹)C(O)—”. Similarly, a moiety

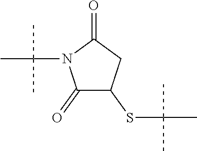

### May be Attached to Two Moieties Either as

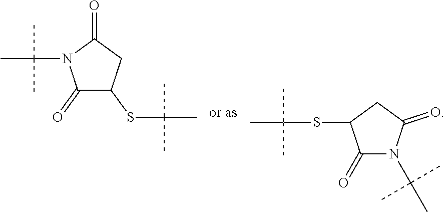

As used herein, the term “functional group” means a group of atoms which can react with other groups of atoms. Functional groups are for example selected from the group consisting of carboxylic acid, primary or secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “pharmaceutically acceptable salt(s) thereof” refers to salts that retain the biological effectiveness or properties of the compound and that typically are not biologically or otherwise undesirable. In certain embodiments, the compound is capable of forming acid/or base salts by virtue of the presence of amino and/or carboxylic functional groups or groups similar thereto. Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethanedisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulfate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, subsalicylate, tartrate, tosylate and trifluoroacetate salts. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, or tromethamine. The pharmaceutically acceptable salts can be synthesized from a parent compound, a basic or acidic moiety, by conventional chemical methods.

The term “pharmaceutically acceptable” means a substance that does not cause harm when administered to a patient and in certain embodiments means approved by a regulatory agency, such as the EMA (Europe) and/or the FDA (US) and/or any other national regulatory agency for use in animals, in particular for use in humans.

As used herein the terms “about” or “approx.” in combination with a numerical value is used to indicate a range ranging from and including the numerical value plus and minus no more than 10% of said numerical value. For example, the phrases “about 200” or “approx. 200” is used to mean a range ranging from and including 200+/−10%, i.e. ranging from and including 180 to 220. It is understood that a percentage given as “about 20%” or “approx. 20%” does not mean “20%+/−10%”, i.e. ranging from and including 10 to 30%, but “about 20%” or “approx. 20%” means ranging from and including 18 to 22%, i.e. plus and minus 10% of the numerical value which is 20.

As used herein, the term “polymer” means a molecule comprising repeating structural units, i.e., the monomers, connected by chemical bonds in a linear, circular, branched, crosslinked or dendrimeric way or a combination thereof, which may be of synthetic or biological origin or a combination of both. It is understood that a polymer may also comprise one or more other chemical groups and/or moieties, such as, for example, one or more functional groups. In certain embodiments a soluble polymer has a molecular weight of at least 0.5 kDa, e.g., a molecular weight of at least 1 kDa, a molecular weight of at least 2 kDa, a molecular weight of at least 3 kDa or a molecular weight of at least 5 kDa. If the polymer is soluble, it in certain embodiments has a molecular weight of at most 1000 kDa, such as at most 750 kDa, such as at most 500 kDa, such as at most 300 kDa, such as at most 200 kDa, or such as at most 100 kDa. It is understood that for water-insoluble polymers, such as hydrogels, no meaningful molecular weight ranges can be provided. It is understood that also a peptide or protein is a polymer in which the amino acids are the repeating structural units, even though the side chains of each amino acid may be different.

As used herein, the term “polymeric” means a reagent or a moiety comprising one or more polymers or polymer moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

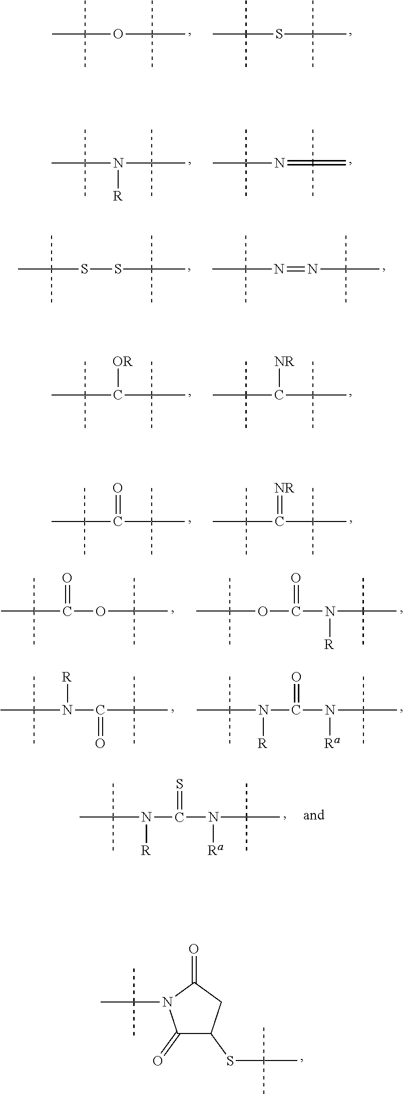

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e., to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lie. An integer for “x” given as “about x” means that the arithmetic mean numbers of monomers lie in a range of integers of x+/−10%, in certain embodiments x+/−8%, in certain embodiments x+/−5% and in certain embodiments x+/−2%.

As used herein, the term “number average molecular weight” means the ordinary arithmetic mean of the molecular weights of the individual polymers.

As used herein the term “water-soluble” with reference to the compound of the present invention means that at least 1 g of the compound may be dissolved in one liter of water at 20° C. to form a homogeneous solution. Accordingly, the term “water-insoluble” with reference to the compound means that less than 1 g of the compound may be dissolved in one liter of water at 20° C. to form a homogeneous solution.

As used herein, the term “PEG-based” in relation to a moiety or reagent means that said moiety or reagent comprises PEG. In certain embodiments a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, such as at least 95%. The remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

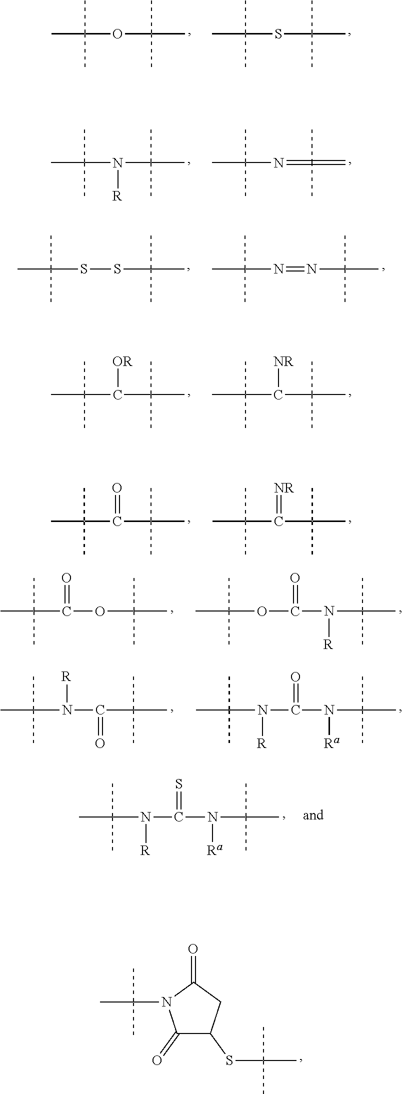

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The term “hyaluronic acid-based” is used accordingly.

The term “substituted” as used herein means that one or more —H atom(s) of a molecule or moiety are replaced by a different atom or a group of atoms, which are referred to as “substituent”.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—S—, —N(R^{x3})—, —OC(OR^{x3})(R^{x3a})—, —N(R^{x3})C(O)N(R^{x3a})—, and —OC(O)N(R^{x3})—; —R^{x1}, —R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4} is independently selected from the group consisting of —H and C₁₋₆ alkyl; wherein C₁₋₆ alkyl is optionally substituted with one or more halogen, which are the same or different.

In certain embodiments the one or more further optional substituents are independently of each other selected from the group consisting of halogen, —CN, —COOR^{x1}, —OW, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl; In certain embodiments the one or more further optional substituents are independently of each other selected from the group consisting of halogen, —CN, —COOR^{x1}, —OW, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —S^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{1xb}), —OC(O)N(R^{x1}R^{1xa}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x2}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different.

In certain embodiments a maximum of 6—H atoms of an optionally substituted molecule are independently replaced by a substituent, e.g. 5—H atoms are independently replaced by a substituent, 4—H atoms are independently replaced by a substituent, 3—H atoms are independently replaced by a substituent, 2—H atoms are independently replaced by a substituent, or 1—H atom is replaced by a substituent.

The term “interrupted” means that a moiety is inserted between two carbon atoms or—if the insertion is at one of the moiety's ends—between a carbon or heteroatom and a hydrogen atom, in certain embodiments between a carbon and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “nucleophile” refers to a reagent or functional group that forms a bond to its reaction partner, i.e., the electrophile by donating both bonding electrons.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)—and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur.

Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

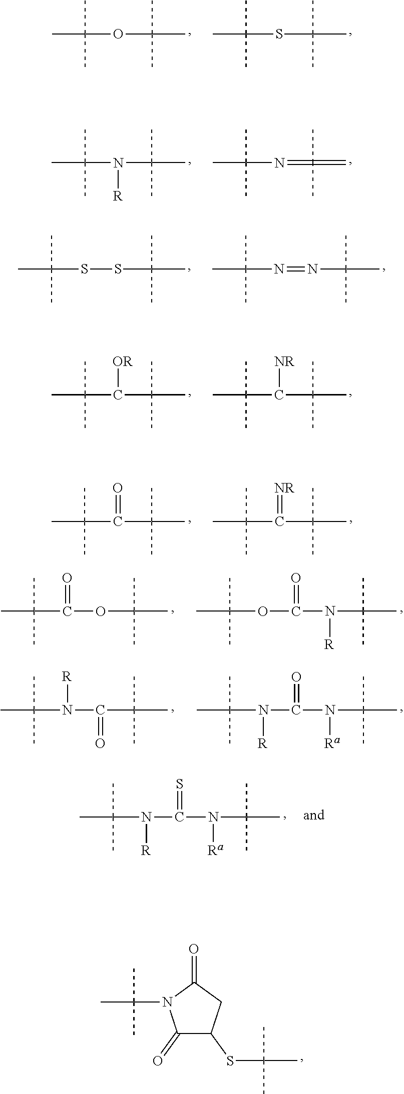

wherein
dashed lines indicate attachment to the remainder of the moiety or reagent; and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

As used herein, the term “C₃₋₁₀ cycloalkyl” means a cyclic alkyl chain having 3 to carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycyclyl” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments an 8- to 30-membered carbopolycyclyl means a cyclic moiety of two, three, four or five rings, in certain embodiments of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycyclyl” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)— and —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

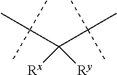

means that R^{x} and R^{y} form the following structure:

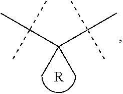

wherein R is C₃₋₁₀ cycloalkyl or 3- to 10-membered heterocyclyl.

It is also understood that the phrase “the pair R/R^{y} is joint together with the atoms to which they are attached to form a ring A” in relation with a moiety of the structure

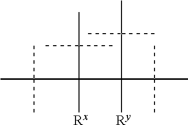

means that R^{x} and R^{y} form the following structure:

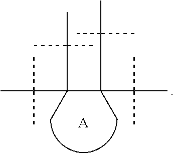

As used herein, “halogen” means fluoro, chloro, bromo or iodo. In certain embodiments halogen is fluoro or chloro.

In general, the term “comprise” or “comprising” also encompasses “consist of” or “consisting of”.

In certain embodiments the compound is of formula (Ia). In certain embodiments x of formula (Ia) is 1. In certain embodiments x of formula (Ia) is 2. In certain embodiments x of formula (Ia) is 3. In certain embodiments x of formula (Ia) is 4. In certain embodiments x of formula (Ia) is 5. In certain embodiments x of formula (Ia) is 6. In certain embodiments x of formula (Ia) is 7. In certain embodiments x of formula (Ia) is 8. In certain embodiments x of formula (Ia) is 9. In certain embodiments x of formula (Ia) is 10. In certain embodiments x of formula (Ia) is 11. In certain embodiments x of formula (Ia) is 12. In certain embodiments x of formula (Ia) is 13. In certain embodiments x of formula (Ia) is 14. In certain embodiments x of formula (Ia) is 15. In certain embodiments x of formula (Ia) is 16. In certain embodiments x of formula (Ia) is 17. In certain embodiments x of formula (Ia) is 18. In certain embodiments x of formula (Ia) is 19. In certain embodiments x of formula (Ia) is 20. In certain embodiments x of formula (Ia) is 21. In certain embodiments x of formula (Ia) is 22. In certain embodiments x of formula (Ia) is 23. In certain embodiments x of formula (Ia) is 24. In certain embodiments x of formula (Ia) is 25.

In certain embodiments the compound is of formula (Ia) with x=1.

In certain embodiments the compound is of formula (Ib). In certain embodiments y of formula (Ib) is 1. In certain embodiments y of formula (Ib) is 2. In certain embodiments y of formula (Ib) is 3. In certain embodiments y of formula (Ib) is 4. In certain embodiments y of formula (Ib) is 5. In certain embodiments y of formula (Ib) is 6. In certain embodiments y of formula (Ib) is 7. In certain embodiments y of formula (Ib) is 8. In certain embodiments y of formula (Ib) is 9. In certain embodiments y of formula (Ib) is 10. In certain embodiments y of formula (Ib) is 11. In certain embodiments y of formula (Ib) is 12. In certain embodiments y of formula (Ib) is 13. In certain embodiments y of formula (Ib) is 14. In certain embodiments y of formula (Ib) is 15. In certain embodiments y of formula (Ib) is 16. In certain embodiments y of formula (Ib) is 17. In certain embodiments y of formula (Ib) is 18. In certain embodiments y of formula (Ib) is 19. In certain embodiments y of formula (Ib) is 20. In certain embodiments y of formula (Ib) is 21. In certain embodiments y of formula (Ib) is 22. In certain embodiments y of formula (Ib) is 23. In certain embodiments y of formula (Ib) is 24. In certain embodiments y of formula (Ib) is 25.

In certain embodiments the compound is of formula (Ib) with y=2.

In certain embodiments -D- is a drug moiety selected from the group consisting of small molecule drug moieties, medium size molecule drug moieties, oligonucleotide drug moieties, peptide nucleic acid drug moieties, peptide drug moieties and protein drug moieties.

In certain embodiments -D- is a peptide drug moiety. In certain embodiments -D- is a small molecule drug moiety. In certain embodiments -D- is a medium size drug moiety. In certain embodiments -D- is an oligonucleotide drug moiety. In certain embodiments -D- is a peptide nucleic acid drug moiety. In certain embodiments -D- is a protein drug moiety.

In certain embodiments -D- or -D-AB² is a GLP-1 receptor agonist moiety. Accordingly, the conjugate of the present invention may be a GLP-1 receptor agonist conjugate.

In certain embodiments -D- or -D-AB² is a mono agonist of the GLP-1 receptor, i.e., only activates the GLP-1 receptor.

In certain embodiments -D- or -D-AB² is an agonist of the GLP-1 receptor and an agonist of a further receptor, i.e., -D- or -D-AB² is a dual GLP-1 receptor agonist. Such further receptor may be selected from the group consisting of the GIP receptor, the GCG receptor, an amylin receptor, a PYY receptor and the GLP-2 receptor.

In certain embodiments -D- or -D-AB² is an agonist of the GLP-1 receptor and of the GIP receptor. In certain embodiments -D- or -D-AB² is an agonist of the GLP-1 receptor and of the GCG receptor. In certain embodiments -D- or -D-AB² is an agonist of the GLP-1 receptor and of an amylin receptor. In certain embodiments -D- or -D-AB² is an agonist of the GLP-1 receptor and of a PYY receptor. In certain embodiments -D- or -D-AB² is an agonist of the GLP-1 receptor and of the GLP-2 receptor.

In certain embodiments -D- or -D-AB² is an agonist of the GLP-1 receptor and growth/differentiation factor 15 (GDF15). In certain embodiments -D- or -D-AB² is an agonist of the GLP-1 receptor and fibroblast growth factor 21 (FGF21).

In certain embodiments -D- or -D-AB² is an agonist of the GLP-1 receptor and an agonist of two further receptors, i.e., -D- or -D-AB² is a triple GLP-1 receptor agonist. These further receptors are in certain embodiments selected from the group consisting of the GIP receptor (GIPR), the GCG receptor (GCGR), an amylin receptor, a PYY receptor (PYYR) and the GLP-2 receptor (GLP2R).

In certain embodiments -D- or -D-AB² is a triple GLP-1 receptor agonist that activates the GLP-1 receptor, the GIP receptor and the GCG receptor. In certain embodiments -D- or -D-AB² is a triple GLP-1 receptor agonist that activates the GLP-1 receptor, the GIP receptor and an amylin receptor. In certain embodiments -D- or -D-AB² is a triple GLP-1 receptor agonist that activates the GLP-1 receptor, the GIP receptor and the PYY receptor. In certain embodiments -D- or -D-AB² is a triple GLP-1 receptor agonist that activates the GLP-1 receptor, the GIP receptor and the GLP-2 receptor. In certain embodiments -D- or -D-AB² is a triple GLP-1 receptor agonist that activates the GLP-1 receptor, the GCG receptor and an amylin receptor. In certain embodiments -D- or -D-AB² is a triple GLP-1 receptor agonist that activates the GLP-1 receptor, the GCG receptor and a PYY receptor. In certain embodiments -D- or -D-AB² is a triple GLP-1 receptor agonist that activates the GLP-1 receptor, the GCG receptor and the GLP-2 receptor. In certain embodiments -D- or -D-AB² is a triple GLP-1 receptor agonist that activates the GLP-1 receptor, an amylin receptor and a PYY receptor. In certain embodiments -D- or -D-AB² is a triple GLP-1 receptor agonist that activates the GLP-1 receptor, an amylin receptor and the GLP-2 receptor. In certain embodiments -D- or -D-AB² is a triple GLP-1 receptor agonist that activates the GLP-1 receptor, a PYY receptor and the GLP-2 receptor.

In certain embodiments -D- is a human GLP-1 of SEQ ID NO:1:

HAEGTFTSDVSSYLEGQAAKEFIAWLVKGRG.

In certain embodiments -D- is human GLP-1 analog of SEQ ID NO:1, which peptide sequence may comprise one or more amino acid changes compared to SEQ ID NO: 1. Such amino acid changes may be the addition of one or more amino acid residues, the deletion of one or more amino acid residues, the substitution of one or more amino acid residues or may be any combination thereof. Such amino acid change may be at the N-terminus, the C-terminus and/or at an internal site of the GLP-1 of SEQ ID NO:1. In certain embodiments such human GLP-1 analog has a maximum of 3 amino acid changes compared to SEQ ID NO:1, i.e., a maximum of three amino acids are added to, deleted from or substituted compared to the sequence of SEQ ID NO:1.

### In Certain Embodiments -D- has the Sequence

(SEQ ID NO: 2)
HX₁EGTFTSDVSSYLEGQAAKEFIAWLVRGRG,

wherein X₁ is 2-aminoisobutyric acid (Aib).

### In Certain Embodiments -D- has the Sequence

(SEQ ID NO: 3)
HX₁EGTFTSDVSSYLEGQAAKEFIAWLVRGRG,

wherein X₁ is Aib and the C-terminal glycine, i.e., the glycine at position 31, is amidated as a C-terminal primary amide.
In Certain Embodiments -D- is Exenatide. Exenatide has the Sequence

(SEQ ID NO: 4)
HGEGTFTSDLSKQMEEEAVRLFIEWLKNGGPSSGAPPPS.

### In Certain Embodiments -D- has the Sequence

(SEQ ID NO: 5)
HGEGTFTSDLSKQMEEEAVRLFIEWLKNGGPSSGAPPPS,

wherein the C-terminal serine, i.e., the serine at position 39, is amidated as a C-terminal primary amide.
In Certain Embodiments -D- is Lixisenatide. Lixisenatide has the Sequence

(SEQ ID NO: 6)
HGEGTFTSDLSKQMEEEAVRLFIEWLKNGGPSSGAPPSKKKKKK,

wherein the C-terminal lysine, i.e., the lysine at position 44, is amidated as a C-terminal primary amide.

### In Certain Embodiments -D- has the Sequence

(SEQ ID NO: 7)
HGEGTFTSDLSKQMEEEAVRLFIEWLKNGGPSSGAPPSKKKKKK.

### In Certain Embodiments -D- has the Sequence

(SEQ ID NO: 8)
HX₁EGTFTSDLSKQX₂EEEAVRLFIEWLKQGGPSSGAPPPC,

wherein X₁ is D-alanine and X₂ is norleucine (Nle).
In certain embodiments -D- has the sequence

(SEQ ID NO: 9)
HX₁EGTFTSDLSKQX₂EEEAVRLFIEWLKQGGPSSGAPPPC,

wherein X₁ is D-alanine, X₂ is Nle and the C-terminal cysteine, i.e., the cysteine at position 39, is amidated as a C-terminal primary amide.
In Certain Embodiments -D- is PEG-Loxenatide. PEG-Loxenatide has the Sequence

(SEQ ID NO: 10)
HX₁EGTFTSDLSKQX₂EEEAVRLFIEWLKQGGPSSGAPPPC,

wherein X₁ is D-alanine; X₂ is Nle; the cysteine at position 39 is chemically modified through conjugation to the thiol group of the cysteine side-chain with

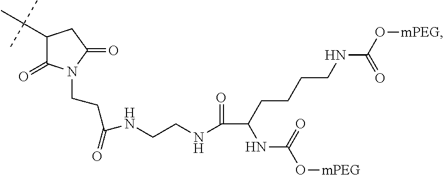

wherein
the dashed line indicates attachment to the thiol group of the cysteine side chain of the cysteine at position 39, and each mPEG is methoxypoly(ethylene glycol) with a molecular weight of approx. 20 kDa.

### In Certain Embodiments -D- has the Sequence

(SEQ ID NO: 11)
HX₁EGTFTSDLSKQX₂EEEAVRLFIEWLKQGGPSSGAPPPC,

wherein X₁ is D-alanine; X₂ is Nle; the cysteine at position 39 is chemically modified through conjugation to the thiol group of the cysteine side-chain with

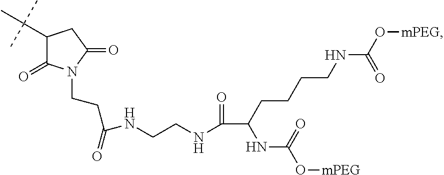

wherein
the dashed line indicates attachment to the thiol group of the cysteine side chain of the cysteine at position 39, each mPEG is methoxypoly(ethylene glycol) with a molecular weight of approx. 20 kDa, and the C-terminal cysteine, i.e., the cysteine at position 39, is amidated as a C-terminal primary amide.

### In Certain Embodiments -D- has the Sequence

(SEQ ID NO: 12)
HAEGTFTSDVSSYLEGQAAKEFIAWLVRGRG.

### In Certain Embodiments -D- has the Sequence

(SEQ ID NO: 13)
HAEGTFTSDVSSYLEGQAAKEFIAWLVRGRG,

and the C-terminal glycine, i.e., the glycine at position 31, is amidated as a C-terminal primary amide.

### In Certain Embodiments -D- has the Sequence

(SEQ ID NO: 14)
HVEGTFTSDVSSYLEEQAAREFIKWLVRGRG.

### In Certain Embodiments -D- has the Sequence

(SEQ ID NO: 15)
HVEGTFTSDVSSYLEEQAAREFIKWLVRGRG,

and the C-terminal glycine, i.e., the glycine at position 31, is amidated as a C-terminal primary amide.

### In Certain Embodiments -D- has the Sequence

(SEQ ID NO: 16)
HGEGTFTSDVSSYLEGQAAKEFIAWLVRGRG.

### In Certain Embodiments -D- has the Sequence

(SEQ ID NO: 17)
HGEGTFTSDVSSYLEGQAAKEFIAWLVRGRG,

and the C-terminal glycine, i.e., the glycine at position 31, is amidated as a C-terminal primary amide.

### In Certain Embodiments -D- has the Sequence

(SEQ ID NO: 18)
HX₁EGTFTSDVSSYLEGQAAKEFIAWLVRGRGL,

wherein X₁ is Aib.

### In Certain Embodiments -D- has the Sequence

(SEQ ID NO: 19)
HX₁EGTFTSDVSSYLEGQAAKEFIAWLVRGRGL,

wherein X₁ is Aib and the C-terminal leucine, i.e., the leucine at position 32, is amidated as a C-terminal primary amide.

### In Certain Embodiments -D- has the Sequence

(SEQ ID NO: 20)
YX₁EGTFTSDYSIX₂LDKIAQKAFVQWLIAGGPSSGAPPPS,

wherein X₁ is Aib, X₂ is Aib and the C-terminal serine, i.e., the serine at position 39, is amidated as a C-terminal primary amide.

### In Certain Embodiments -D- has the Sequence

(SEQ ID NO: 21)
YX₁EGTFTSDYSIX₂LDKIAQKAFVQWLIAGGPSSGAPPPS,

wherein X₁ is Aib and X₂ is Aib.

### In Certain Embodiments -D- has the Sequence

(SEQ ID NO: 22)
HSQGTFTSDKSEYLDSERARDFVAWLEAGG.

### In Certain Embodiments -D- has the Sequence

(SEQ ID NO: 23)
HSQGTFTSDKSEYLDSERARDFVAWLEAGG,

wherein the C-terminal glycine, i.e., the glycine at position 30, is amidated as a C-terminal primary amide.

### In Certain Embodiments -D- has the Sequence

(SEQ ID NO: 24)
HX₁QGTFTSDYSKYLDERAAKDFIKWLESA,

wherein X₁ is 1-amino-cyclobutanecarboxylic acid (Ac4c); and the C-terminal alanine, i.e. the alanine at position 29, is amidated as a C-terminal primary amide.

### In Certain Embodiments -D- has the Sequence

(SEQ ID NO: 25)
HX₁QGTFTSDYSKYLDERAAKDFIKWLESA,

wherein X₁ is 1-amino-cyclobutanecarboxylic acid (Ac4c).

### In Certain Embodiments -D- has the Sequence

(SEQ ID NO: 26)
HX₁QGTFTSDYSKYLDEKAAKEFIQWLLQT,

wherein X₁ is Aib and the glutamic acid at position 16 and the lysine at position 20 are connected via a lactam bridge.

### In Certain Embodiments -D- has the Sequence

(SEQ ID NO: 27)
HX₁QGTFTSDYSKYLDEKAAKEFIQWLLQT,

wherein X₁ is Aib, the glutamic acid at position 16 and the lysine at position 20 are connected via a lactam bridge and the C-terminal threonine, i.e., the threonine at position 29, is amidated as a C-terminal primary amide.

### In Certain Embodiments -D- has the Sequence

(SEQ ID NO: 28)
HX₁QGTFTSDYSKYLDEKKAKEFVEWLLEGGPSSG,

wherein X₁ is Aib and the C-terminal glycine, i.e., the glycine at position 34, is amidated as a C-terminal primary amide.

### In Certain Embodiments -D- has the Sequence

(SEQ ID NO: 29)
HX₁QGTFTSDYSKYLDEKKAKEFVEWLLEGGPSSG,

wherein X₁ is Aib.

### In Certain Embodiments -D- has the Sequence

(SEQ ID NO: 30)
HX₁EGSFTSELATILDKQAARDFIAWLIQHKITD,

wherein X₁ is Aib.

### In Certain Embodiments -D- has the Sequence

(SEQ ID NO: 31)
HX₁EGSFTSELATILDKQAARDFIAWLIQHKITD,

wherein X₁ is Aib and the C-terminal aspartic acid, i.e., the aspartic acid at position 33, is amidated as a C-terminal primary amide.

### In Certain Embodiments -D- has the Sequence

(SEQ ID NO: 32)
YX₁QGTFTSDYSIX₂LDKKAQX₃AFIEYLLEGGPSSGAPPPS,

wherein X₁ is Aib, X₂ is α-methyl-leucine (αMeL), X₃ is Aib; and the C-terminal serine, i.e. the serine at position 39, is amidated as a C-terminal primary amide.

### In Certain Embodiments -D- has the Sequence

(SEQ ID NO: 33)
YX₁QGTFTSDYSIX₂LDKKAQX₃AFIEYLLEGGPSSGAPPPS,

wherein X₁ is Aib, X₂ is α-methyl-leucine (αMeL) and X₃ is Aib.
In certain embodiments -D- has the sequence

(SEQ ID NO: 54)
HX₁EGTFTSDVSSYLEEEAAKEFIAWLVRGGPSSGAPPPSK,

wherein X₁ is Aib.

### In Certain Embodiments -D- has the Sequence

(SEQ ID NO: 55)
HX₁EGTFTSDVSSYLEEQAAKEFIAWLVRGGG,

wherein X₁ is Aib.

### In Certain Embodiments -D- has the Sequence

(SEQ ID NO: 56)
HX₁EGTFTSDVSSYLEEQAAKEFIAWLVRGGGGAQPGAQPGAQPGAQPG
AQPGAQPGAQPGAQPGAQPGQKP,

wherein X₁ is Aib.

### In Certain Embodiments -D- has the Sequence

(SEQ ID NO: 57)
YX₁EGTFTSDYSIX₂LDKIAQKAFVQWLIAGGPSSGAPPPS,

wherein X₁ and X₂ are both Aib.

In certain embodiments -D- is a drug selected from the group consisting of insulins; amylin and amylin/calcitonin; PYY; GIP; MSH; C5a binders; GDF15; PCSK9 I; immune stimulants; urocortin2; MIC-1; IL-1R antagonists; leptin; gastrin; glucagon; exendin-4; GLP-1; GLP-2; and GIP.

In certain embodiments -D- is a drug moiety selected from the group consisting of insulin; insulin analogues; amylin; dual amylin/calcitonin agonists; PYY; GIP; MSH; C₅ inhibitors or modulators; GDF15; PCSK9 inhibitors; immune stimulants; urocortin II; MIC-1; IL-1R antagonists; leptin; gastrin; glucagon; oxyntomodulin; neurokinin A; tachykinin/neurokinin receptor 2 (NK2R) agonists; neurokinin receptor (NKR) agonists and GLP-2.

In certain embodiments -D- is an insulin, such as insulin detemir, insulin degludec or insulin. In certain embodiments -D- is amylin or amylin/calcitonin, such as for example cagrilintide. In certain embodiments -D- is PYY, such as NNC0165-1875. In certain embodiments -D- is GIP. In certain embodiments -D- is MSH. In certain embodiments -D- is a C₅a binder, such as zilucoplan. In certain embodiments -D- is GDF15, such as NN LA-GDF15. In certain embodiments -D- is PCSK9 i. In certain embodiments -D- is an immune stimulant, such as romurtide or mifamurtide. In certain embodiments -D- is muramyl dipeptide. In certain embodiments -D- is urocortin2. In certain embodiments -D- is MIC-1. In certain embodiments -D- is an IL-1R antagonist. In certain embodiments -D- is leptin. In certain embodiments -D- is gastrin.

In certain embodiments -D- is selected from the list consisting of growth hormones, such as human growth hormone; FGF21; EGF(a); and coagulations factors.

In certain embodiments -D- is a growth hormone, such as a human growth hormone, such as somapacitan. In certain embodiments -D- is FGF21, such as NNCO194 0499. In certain embodiments -D- is EGF(a). In certain embodiments -D- is a coagulation factor.

In certain embodiments -D- is selected from cytotoxic small molecule drugs; chemotherapy small molecule drugs; and immune activating small molecule drugs.

In certain embodiments -D- is a cytotoxic small molecule drug. In certain embodiments -D- is a chemotherapy small molecule drug. In certain embodiments -D- is and immune activating small molecule drug, such as telratolimod.

In certain embodiments -D- is paclitaxel. In certain embodiments -D- is doxorubicin. In certain embodiments -D- is 5-FU.

In certain embodiments -D- is a PTH moiety.

A moiety -AB¹ is conjugated to -D- via -L²-L¹-, wherein -L²- is conjugated to -AB¹ and -L¹- is conjugated to -D-. Upon cleavage of the linkage between -L¹- and -D- the moiety -AB¹ remains conjugated to -L²-. A moiety -AB² is directly conjugated to -D-, such as through a stable linkage. This means that upon cleavage of the linkage between -L¹- and -D- a conjugate H-D-AB² is released, wherein H—is hydrogen. Accordingly, the moiety “H-D-AB²” is also referred to as the corresponding free drug of a moiety -D-AB².

A moiety -AB¹ and -AB² binds to albumin, such as human albumin, under physiological conditions (aqueous buffer pH 7.4, 37.4° C.).

In certain embodiments -AB¹ and -AB² have a different structure. In certain embodiments -AB¹ and -AB² have the same structure.

Each -AB¹ and -AB² may independently be a fatty acid-based albumin-binding moiety or a peptidic albumin binding moiety.

In certain embodiments -AB¹ and -AB² are independently a fatty acid-based albumin-binding moiety. In certain embodiments -AB¹ and -AB² are both a fatty acid-based albumin-binding moiety and have the same structure. In certain embodiments -AB¹ and -AB² are both a fatty acid-based albumin-binding moiety but have a different structure.

In certain embodiments -AB¹ and/or -AB² are independently of formula (A):

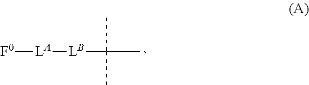

wherein
the dashed line indicates attachment to -L²- or -D-, respectively; —F⁰ is of formula (a-1)

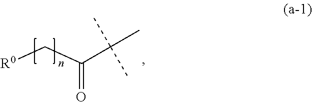

wherein
the dashed line indicates attachment to -L^{A}-;
—R⁰ is selected from the group consisting of —CR¹R^{1a}R^{1b}, —COOR¹,

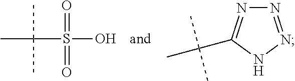

—R¹, —R^{1a} and —R^{1b} are selected from the group consisting of —H, methyl, ethyl, propyl and isopropyl;
n is an integer ranging from and including 8, 9, 10, 11, 12, 13, 14, 15, 16, 17, 18, 19, 20, 21 and 22;
-L^{A}- is absent or is of formula (a-2)

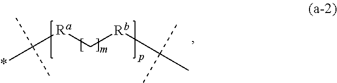

wherein
the unmarked dashed line indicates attachment to -L^{B}-;
the dashed line marked with the asterisk indicates attachment to —F⁰;
—R^{a}—is selected from the group consisting of

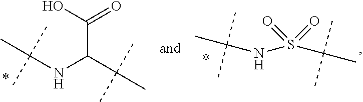

wherein
the dashed line marked with the asterisk indicates attachment to —F⁰;
the unmarked dashed line indicates attachment to the remainder of -L^{A}-.
—R^{b}—is

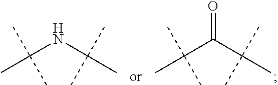

m is an integer selected from the group consisting of 1, 2, 3, 4, 5, 6, 7, 8, 9 and 10
p is an integer selected from the group consisting of 1, 2, 3, 4, 5, 6, 7, 8, 9 and 10;
-L^{B}- is absent or is of formula (a-3)

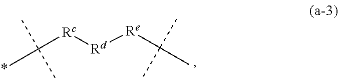

wherein
the unmarked dashed line indicates attachment to -L²- or -D-;
the dashed line marked with the asterisk indicates attachment to -L^{A};
—R^{c}—is

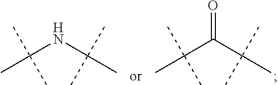

—R^{d}—is selected from the group consisting of C₁₋₅₀ alkyl, C₂₋₅₀ alkenyl and C₂₋₅₀ alkynyl, wherein C₁₋₅₀ alkyl, C₂₋₅₀ alkenyl and C₂₋₅₀ alkynyl may be substituted with one or more —R¹, which may be the same or different, and which C₁₋₅₀ alkyl, C₂₋₅₀ alkenyl or C₂₋₅₀ alkynyl may be interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- may independently be substituted with one or more —R¹, which may be the same or different;
   each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N (R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl may be substituted with one or more halogen, which may be the same or different; and
—R^{e}—is selected from the group consisting of —CH₂—,

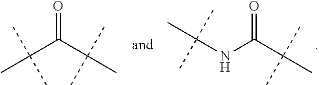

If —R^{b}—is

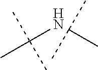

and -L^{B}- is not absent, then —R^{c}—is

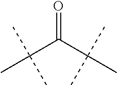

### and if —R^{b}—is

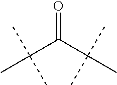

and -L^{B}- is not absent, then —R^{c}—is

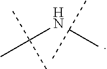

In certain embodiments -L^{A}- is of formula (a-2), i.e., is not absent. In certain embodiments -L^{A}- is absent.

In certain embodiments —R^{a}—is

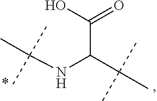

wherein the dashed line marked with the asterisk indicates attachment to —F⁰ and the unmarked dashed line indicates attachment to the remainder of -L^{A}-.

In certain embodiments —R^{a}—is

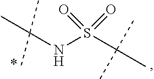

wherein the dashed line marked with the asterisk indicates attachment to —F⁰ and the unmarked dashed line indicates attachment to the remainder of -L^{A}-.

In certain embodiments —R^{b}—is

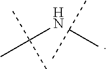

In certain embodiments —R^{b}—is

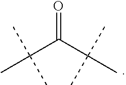

In certain embodiments m of formula (a-2) is 1. In certain embodiments m of formula (a-2) is 2. In certain embodiments m of formula (a-2) is 3. In certain embodiments m of formula (a-2) is 4. In certain embodiments m of formula (a-2) is 5. In certain embodiments m of formula (a-2) is 6. In certain embodiments m of formula (a-2) is 7. In certain embodiments m of formula (a-2) is 8. In certain embodiments m of formula (a-2) is 9. In certain embodiments m of formula (a-2) is 10.

In certain embodiments p of formula (a-2) is 1. In certain embodiments p of formula (a-2) is 2. In certain embodiments p of formula (a-2) is 3. In certain embodiments p of formula (a-2) is 4. In certain embodiments p of formula (a-2) is 5. In certain embodiments p of formula (a-2) is 6. In certain embodiments p of formula (a-2) is 7. In certain embodiments p of formula (a-2) is 8. In certain embodiments p of formula (a-2) is 9. In certain embodiments p of formula (a-2) is 10.

In certain embodiments -L^{B}- is of formula (a-3). In certain embodiments -L^{B}- is absent. If -L^{B}- is absent, the unmarked dashed line in formula (a-2) indicates attachment to -L²- or -D-, respectively.

In certain embodiments —R^{c}—is

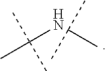

In certain embodiments —R^{c}—is

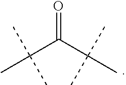

In certain embodiments —R^{e}—is —CH₂—. In certain embodiments —R^{e}—is

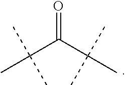

In certain embodiments —R^{e}—is

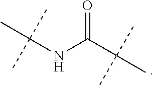

In certain embodiments both -L^{A}- and -L^{B}- are absent. If both -L^{A}- and -L^{B}- are absent, the dashed line in formula (a-1) indicates attachment to -L²- or -D-, respectively.

In certain embodiments —F⁰ is selected from the group consisting of

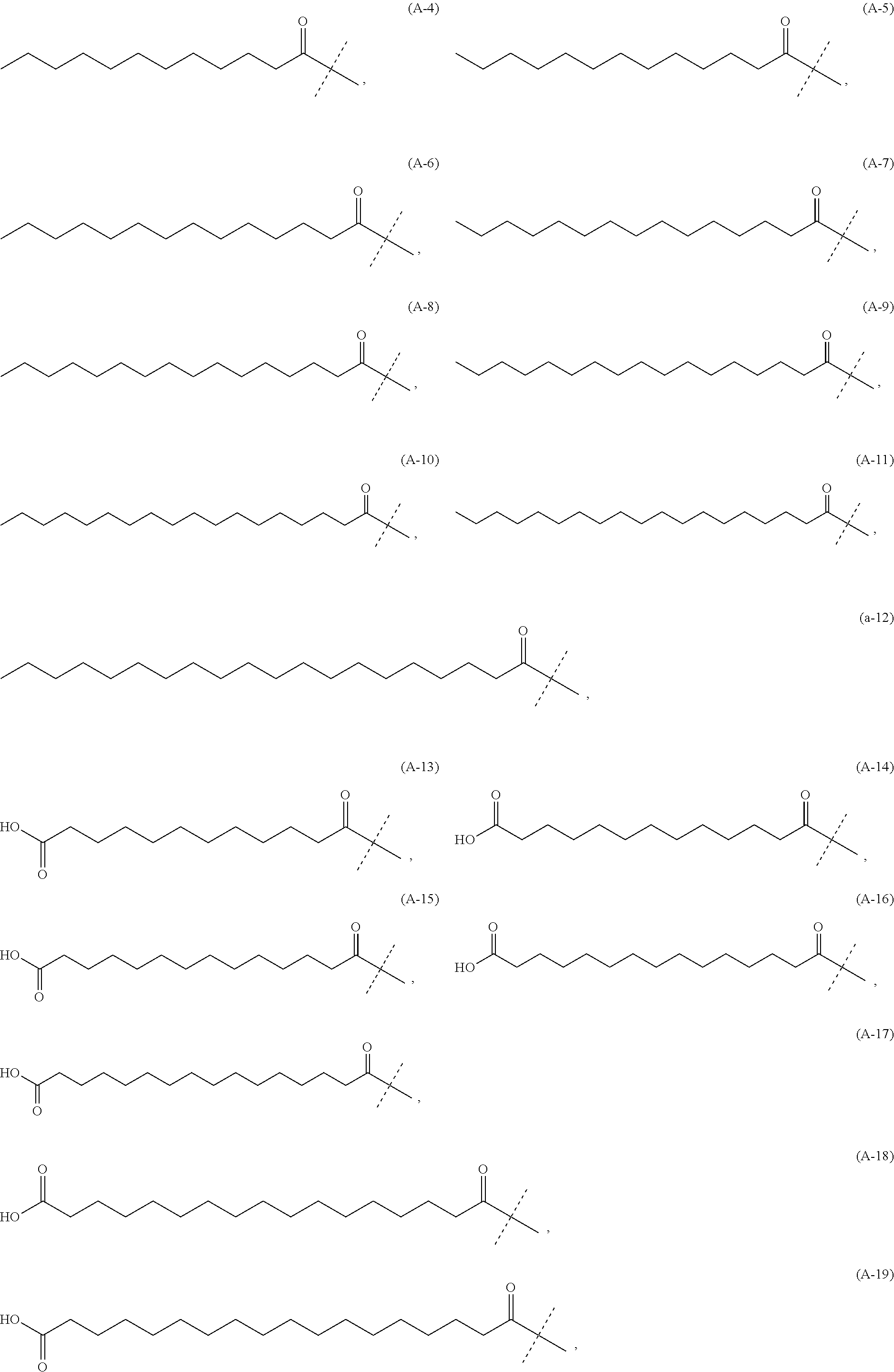

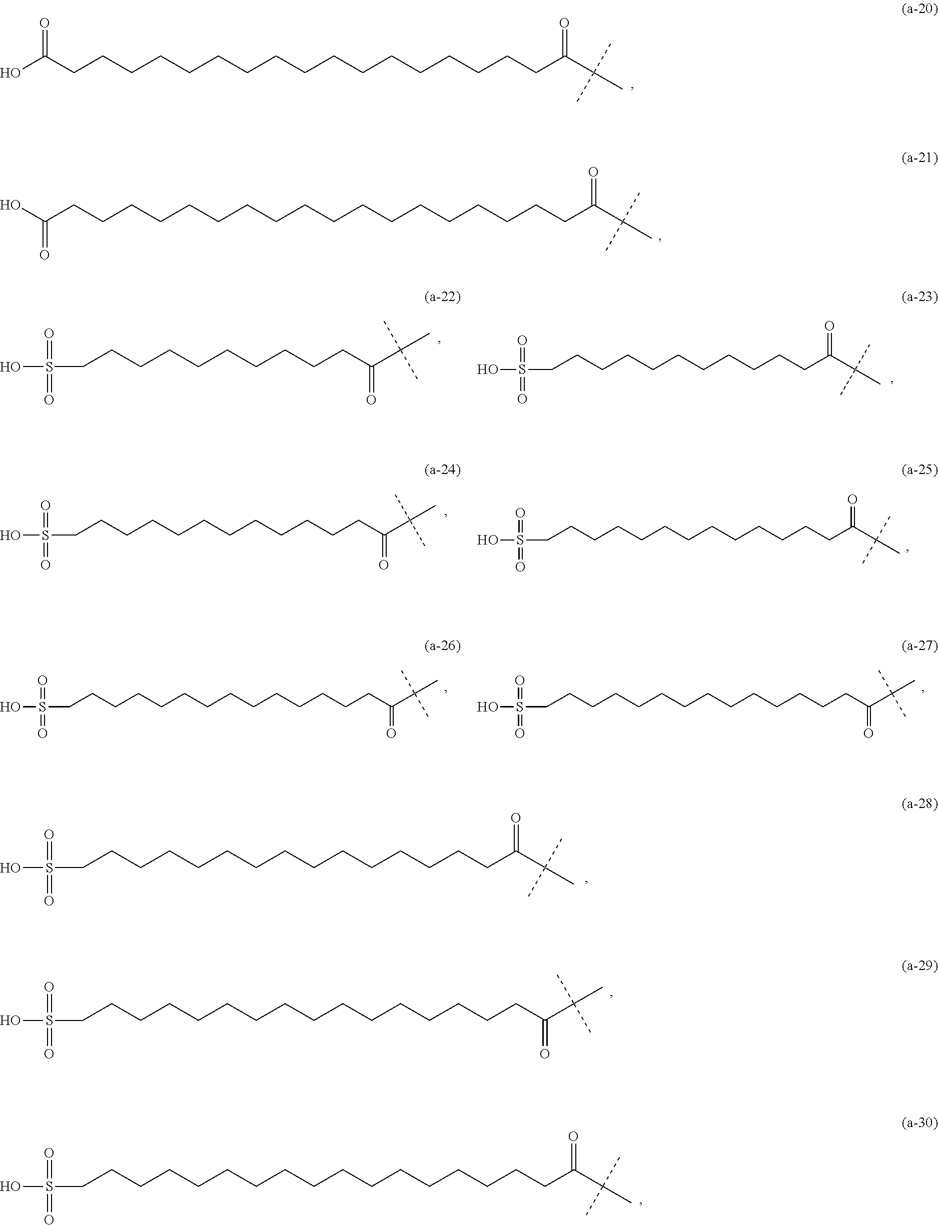

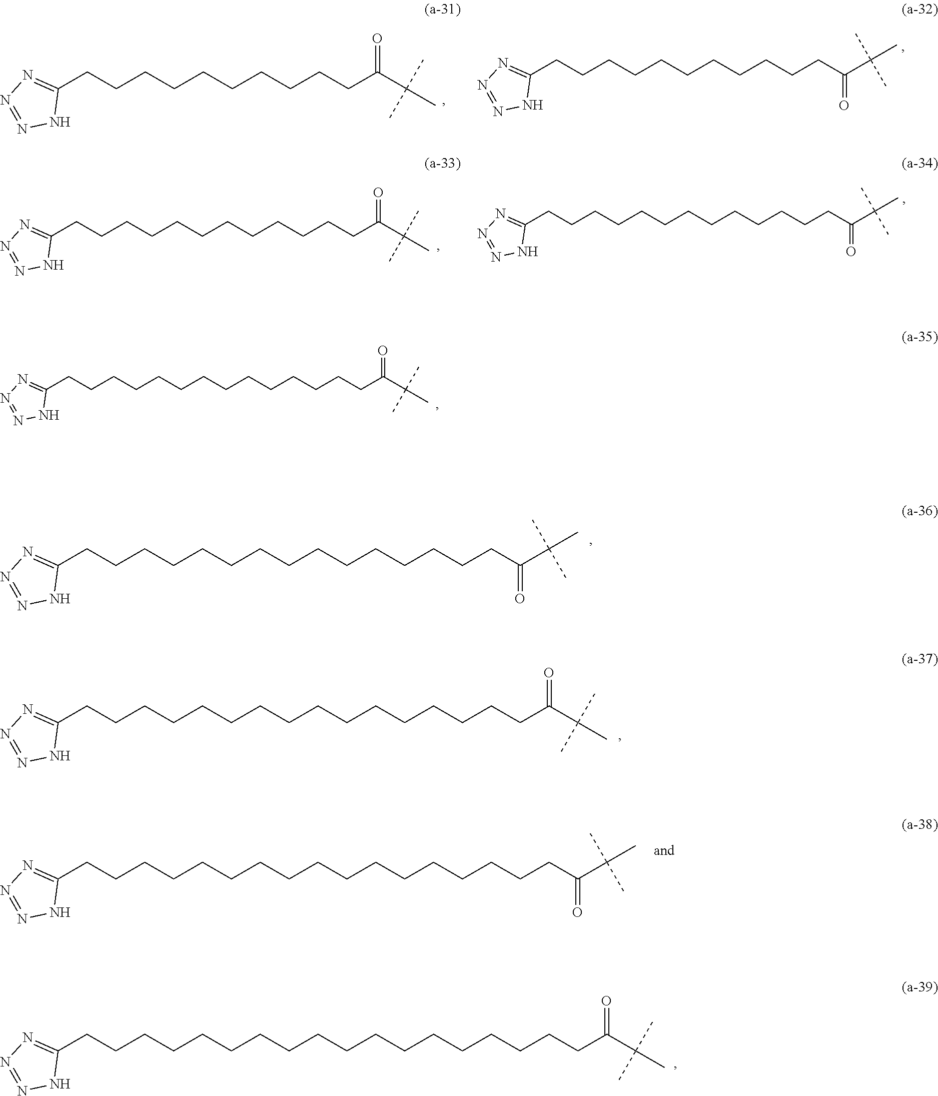

wherein dashed lines indicate attachment to -L^{A}-.

If -L^{A}- is absent, the dashed line in formulas (a-4) to (a-39) indicates attachment to -L^{B}-. If both -L^{A}- and -L^{B}- are absent, the dashed line in formulas (a-4) to (a-39) indicates attachment to -D-.

In certain embodiments —F⁰ is of formula (a-4). In certain embodiments —F⁰ is of formula (a-5). In certain embodiments —F⁰ is of formula (a-6). In certain embodiments —F⁰ is of formula (a-7). In certain embodiments —F⁰ is of formula (a-8). In certain embodiments —F⁰ is of formula (a-9). In certain embodiments —F⁰ is of formula (a-10). In certain embodiments —F⁰ is of formula (a-11). In certain embodiments —F⁰ is of formula (a-12). In certain embodiments —F⁰ is of formula (a-13). In certain embodiments —F⁰ is of formula (a-14). In certain embodiments —F⁰ is of formula (a-15). In certain embodiments —F⁰ is of formula (a-16). In certain embodiments —F⁰ is of formula (a-17). In certain embodiments —F⁰ is of formula (a-18). In certain embodiments —F⁰ is of formula (a-19). In certain embodiments —F⁰ is of formula (a-20). In certain embodiments —F⁰ is of formula (a-21). In certain embodiments —F⁰ is of formula (a-22). In certain embodiments —F⁰ is of formula (a-23). In certain embodiments —F⁰ is of formula (a-24). In certain embodiments —F⁰ is of formula (a-25). In certain embodiments —F⁰ is of formula (a-26). In certain embodiments —F⁰ is of formula (a-27). In certain embodiments —F⁰ is of formula (a-28). In certain embodiments —F⁰ is of formula (a-29). In certain embodiments —F⁰ is of formula (a-30). In certain embodiments —F⁰ is of formula (a-31). In certain embodiments —F⁰ is of formula (a-32). In certain embodiments —F⁰ is of formula (a-33). In certain embodiments —F⁰ is of formula (a-34). In certain embodiments —F⁰ is of formula (a-35). In certain embodiments —F⁰ is of formula (a-36). In certain embodiments —F⁰ is of formula (a-37). In certain embodiments —F⁰ is of formula (a-38). In certain embodiments —F⁰ is of formula (a-39).

In certain embodiments —F⁰ is of formula (a-4) and both -L^{A}- and -L^{B}- are absent. In certain embodiments —F⁰ is of formula (a-5) and both -L^{A}- and -L^{B}- are absent. In certain embodiments —F⁰ is of formula (a-6) and both -L^{A}- and -L^{B}- are absent. In certain embodiments —F⁰ is of formula (a-7) and both -L^{A}- and -L^{B}- are absent. In certain embodiments —F⁰ is of formula (a-8) and both -L^{A}- and -L^{B}- are absent. In certain embodiments —F⁰ is of formula (a-9) and both -L^{A}- and -L^{B}- are absent. In certain embodiments —F⁰ is of formula (a-10) and both -L^{A}- and -L^{B}- are absent. In certain embodiments —F⁰ is of formula (a-11) and both -L^{A}- and -L^{A}- are absent. In certain embodiments —F⁰ is of formula (a-12) and both -L^{A}- and -L^{B}- are absent. In certain embodiments —F⁰ is of formula (a-13) and both -L^{A}- and -L^{B}- are absent. In certain embodiments —F⁰ is of formula (a-14) and both -L^{A}- and -L^{B}- are absent. In certain embodiments —F⁰ is of formula (a-15) and both -L^{A}- and -L^{B}- are absent. In certain embodiments —F⁰ is of formula (a-16) and both -L^{A}- and -L^{B}- are absent. In certain embodiments —F⁰ is of formula (a-17) and both -L^{A}- and -L^{B}- are absent. In certain embodiments —F⁰ is of formula (a-18) and both -L^{A}- and -L^{B}- are absent. In certain embodiments —F⁰ is of formula (a-19) and both -L^{A}- and -L^{B}- are absent. In certain embodiments —F⁰ is of formula (a-20) and both -L^{A}- and -L^{B}- are absent. In certain embodiments —F⁰ is of formula (a-21) and both -L^{A}- and -L^{B}- are absent. In certain embodiments —F⁰ is of formula (a-22) and both -L^{A}- and -L^{B}- are absent. In certain embodiments —F⁰ is of formula (a-23) and both -L^{A}- and -L^{B}- are absent. In certain embodiments —F⁰ is of formula (a-24) and both -L^{A}- and -L^{B}- are absent. In certain embodiments —F⁰ is of formula (a-25) and both -L^{A}- and -L^{B}- are absent. In certain embodiments —F⁰ is of formula (a-26) and both -L^{A}- and -L^{B}- are absent. In certain embodiments —F⁰ is of formula (a-27) and both -L^{A}- and -L^{B}- are absent. In certain embodiments —F⁰ is of formula (a-28) and both -L^{A}- and -L^{B}- are absent. In certain embodiments —F⁰ is of formula (a-29) and both -L^{A}- and -L^{B}- are absent. In certain embodiments —F⁰ is of formula (a-30) and both -L^{A}- and -L^{B}- are absent. In certain embodiments —F⁰ is of formula (a-31) and both -L^{A}- and -L^{B}- are absent. In certain embodiments —F⁰ is of formula (a-32) and both -L^{A}- and -L^{B}- are absent. In certain embodiments —F⁰ is of formula (a-33) and both -L^{A}- and -L^{B}- are absent. In certain embodiments —F⁰ is of formula (a-34) and both -L^{A}- and -L^{B}- are absent. In certain embodiments —F⁰ is of formula (a-35) and both -L^{A}- and -L^{B}- are absent. In certain embodiments —F⁰ is of formula (a-36) and both -L^{A}- and -L^{B}- are absent. In certain embodiments —F⁰ is of formula (a-37) and both -L^{A}- and -L^{B}- are absent. In certain embodiments —F⁰ is of formula (a-38) and both -L^{A}- and -L^{B}- are absent. In certain embodiments —F⁰ is of formula (a-39) and both -L^{A}- and -L^{B}- are absent.

In certain embodiments -L^{A}- is selected from the group consisting of

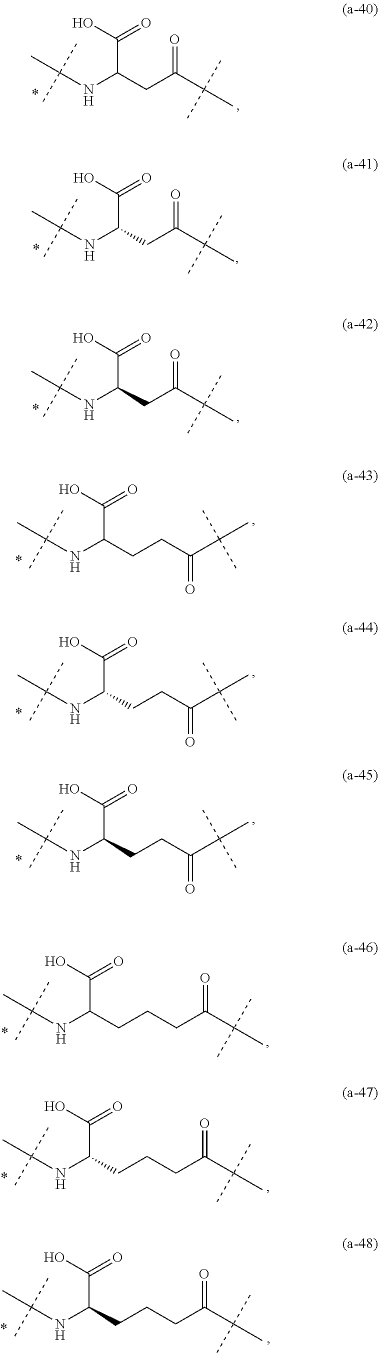

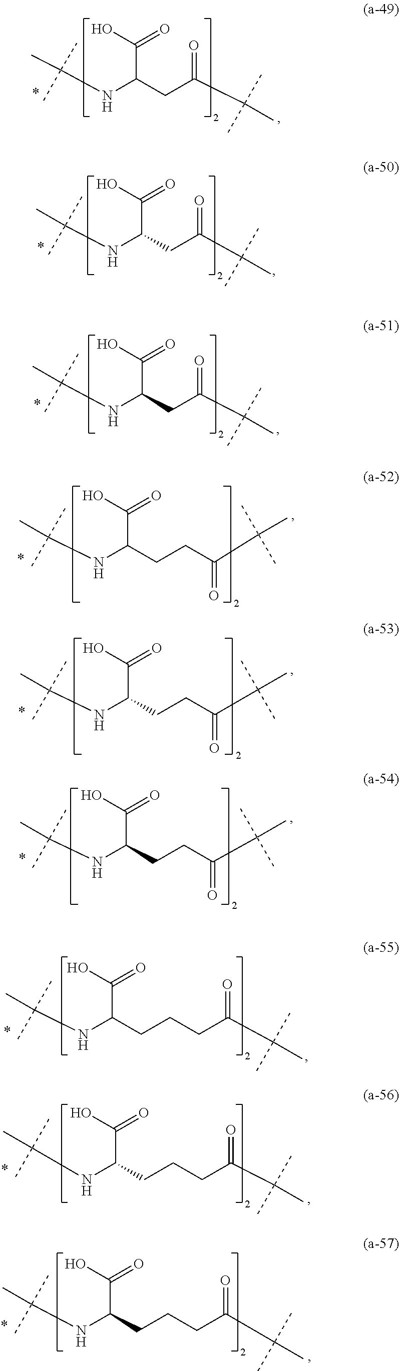

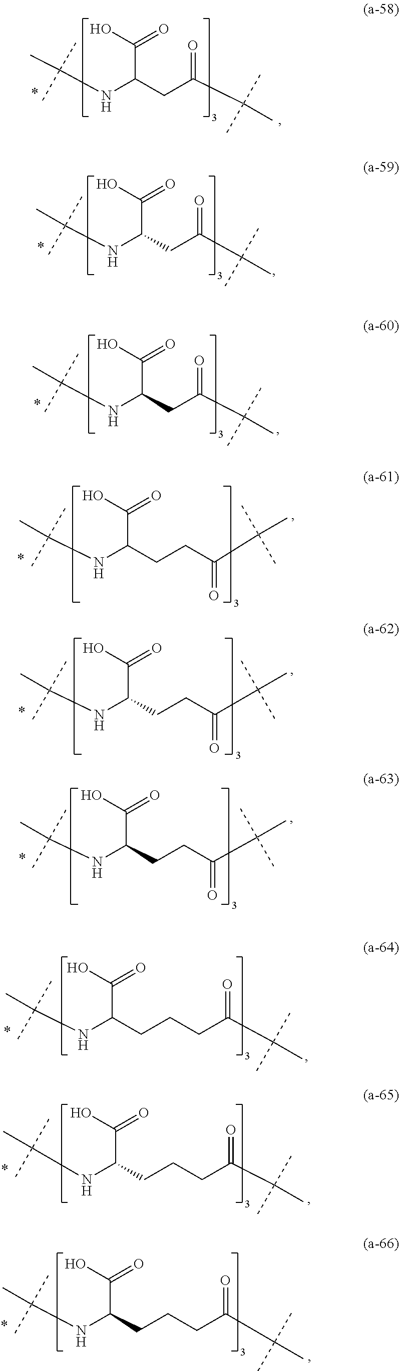

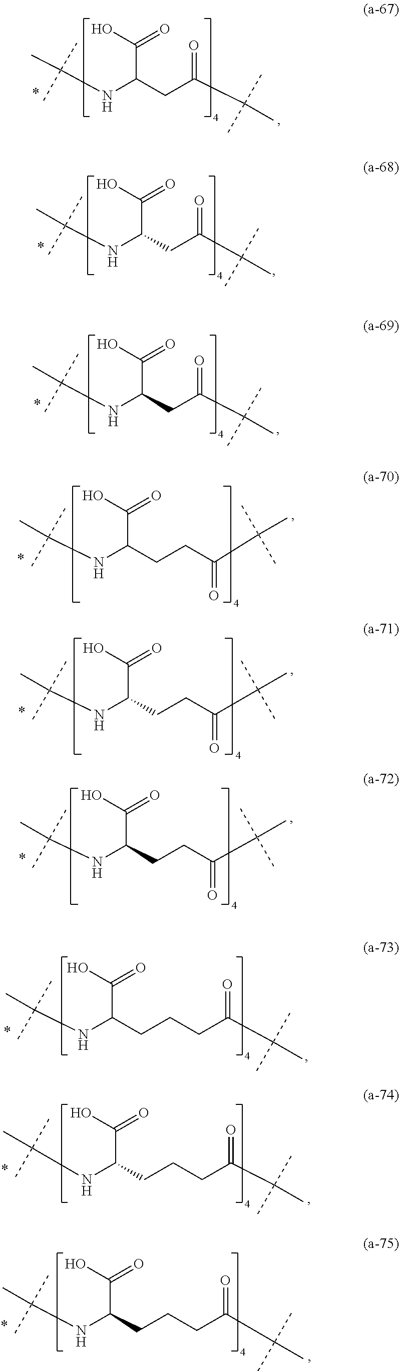

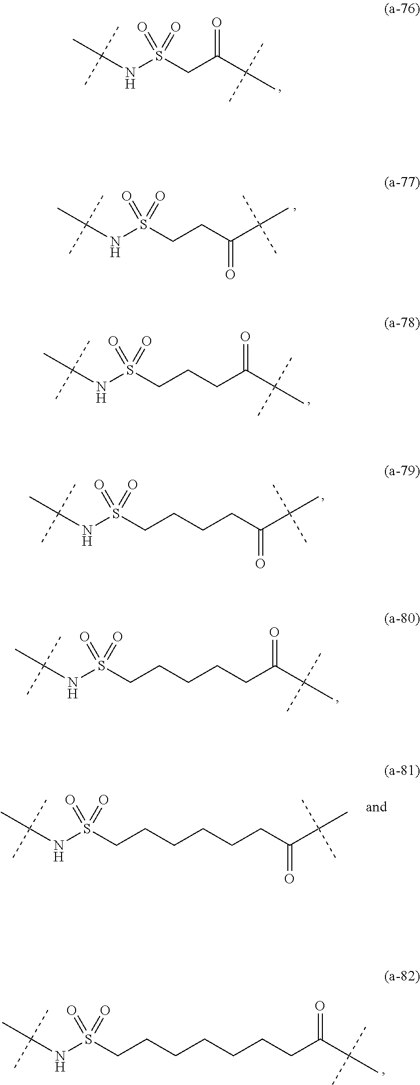

wherein a dashed line marked with an asterisk indicates attachment to —F⁰—and an unmarked dashed line indicates attachment to -L^{B}- If -L^{B}- is absent an unmarked dashed indicates attachment to -D-.

In certain embodiments -L^{A}- is of formula (a-40). In certain embodiments -L^{A}- is of formula (a-41). In certain embodiments -L^{A}- is of formula (a-42). In certain embodiments -L^{A}- is of formula (a-43). In certain embodiments -L^{A}- is of formula (a-44). In certain embodiments -L^{A}- is of formula (a-45). In certain embodiments -L^{A}- is of formula (a-46). In certain embodiments -L^{A}- is of formula (a-47). In certain embodiments -L^{A}- is of formula (a-48). In certain embodiments -L^{A}- is of formula (a-49). In certain embodiments -L^{A}- is of formula (a-50). In certain embodiments -L^{A}- is of formula (a-51). In certain embodiments -L^{A}- is of formula (a-52). In certain embodiments -L^{A}- is of formula (a-53). In certain embodiments -L^{A}- is of formula (a-54). In certain embodiments -L^{A}- is of formula (a-55). In certain embodiments -L^{A}- is of formula (a-56). In certain embodiments -L^{A}- is of formula (a-57). In certain embodiments -L^{A}- is of formula (a-58). In certain embodiments -L^{A}- is of formula (a-59). In certain embodiments -L^{A}- is of formula (a-60). In certain embodiments -L^{A}- is of formula (a-61). In certain embodiments -L^{A}- is of formula (a-62). In certain embodiments -L^{A}- is of formula (a-63). In certain embodiments -L^{A}- is of formula (a-64). In certain embodiments -L^{A}- is of formula (a-65). In certain embodiments -L^{A}- is of formula (a-66). In certain embodiments -L^{A}- is of formula (a-67). In certain embodiments -L^{A}- is of formula (a-68). In certain embodiments -L^{A}- is of formula (a-69). In certain embodiments -L^{A}- is of formula (a-70). In certain embodiments -L^{A}- is of formula (a-71). In certain embodiments -L^{A}- is of formula (a-72). In certain embodiments -L^{A}- is of formula (a-73). In certain embodiments -L^{A}- is of formula (a-74). In certain embodiments -L^{A}- is of formula (a-75). In certain embodiments -L^{A}- is of formula (a-76). In certain embodiments -L^{A}- is of formula (a-77). In certain embodiments -L^{A}- is of formula (a-78). In certain embodiments -L^{A}- is of formula (a-79). In certain embodiments -L^{A}- is of formula (a-80). In certain embodiments -L^{A}- is of formula (a-81). In certain embodiments -L^{A}- is of formula (a-82).

In certain embodiments -L^{B}- is selected from the group consisting of

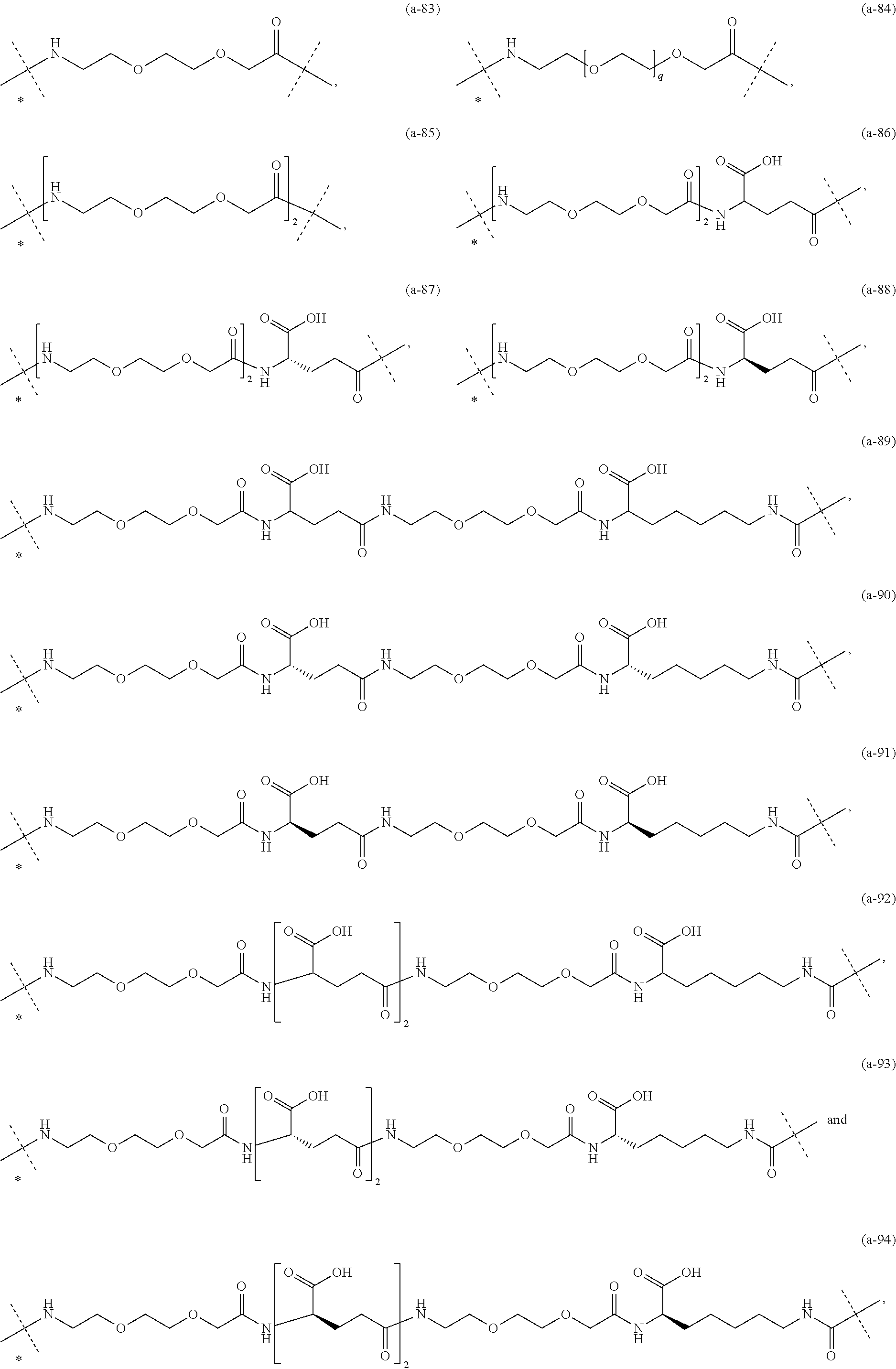

wherein
the dashed line marked with the asterisk indicates attachment to -L^{A}-;
the unmarked dashed line indicates attachment to -L²- or -D-, respectively; and
q is an integer ranging from and including 2 to 50.

If -L^{A}- is absent, the dashed line marked with the asterisk in formulas (a-83) to (a-94) indicates attachment to —F⁰.

In certain embodiments q of formula (a-84) is an integer ranging from and including 3 to 45. In certain embodiments q of formula (a-84) is an integer ranging from and including 4 to 40. In certain embodiments q of formula (a-84) is an integer ranging from and including 5 to 35. In certain embodiments q of formula (a-84) is an integer ranging from and including 6 to 30. In certain embodiments q of formula (a-84) is an integer ranging from and including 7 to 25. In certain embodiments q of formula (a-84) is an integer ranging from and including 10 to 20. In certain embodiments q of formula (a-84) is 23.

In certain embodiments -L^{B}- is of formula (a-83). In certain embodiments -L^{B}- is of formula (a-84). In certain embodiments -L^{B}- is of formula (a-84) with q=23. In certain embodiments -L^{B}- is of formula (a-85). In certain embodiments -L^{B}- is of formula (a-86). In certain embodiments -L^{B}- is of formula (a-87). In certain embodiments -L^{B}- is of formula (a-88). In certain embodiments -L^{B}- is of formula (a-89). In certain embodiments -L^{B}- is of formula (a-90). In certain embodiments -L^{B}- is of formula (a-91). In certain embodiments -L^{B}- is of formula (a-92). In certain embodiments -L^{B}- is of formula (a-93). In certain embodiments -L^{B}- is of formula (a-94).

In certain embodiments -AB¹ and/or -AB² is of formula (i)

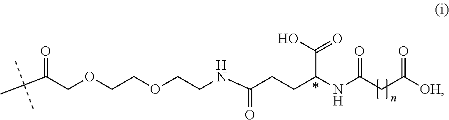

wherein the dashed line indicates attachment to -L²- or -D-, respectively;
n is an integer ranging from and including 14 to 22; and
the stereocenter marked with the asterisk is either in S- or R-configuration.

In certain embodiments -AB¹ and/or -AB² is of formula (i) and n is 14. In certain embodiments -AB¹ and/or -AB² is of formula (i) and n is 15. In certain embodiments -AB¹ and/or -AB² is of formula (i) and n is 16. In certain embodiments -AB¹ and/or -AB² is of formula (i) and n is 17. In certain embodiments -AB¹ and/or -AB² is of formula (i) and n is 18. In certain embodiments -AB¹ and/or -AB² is of formula (i) and n is 19. In certain embodiments -AB¹ and/or -AB² is of formula (i) and n is 20. In certain embodiments -AB¹ and/or -AB² is of formula (i) and n is 21. In certain embodiments -AB¹ and/or -AB² is of formula (i) and n is 22.

In certain embodiments -AB¹ and/or -AB² is of formula (i) and n is 14 and the stereocenter marked with the asterisk is in R-configuration. In certain embodiments -AB¹ and/or -AB² is of formula (i) and n is 15 and the stereocenter marked with the asterisk is in R-configuration. In certain embodiments -AB¹ and/or -AB² is of formula (i) and n is 16 and the stereocenter marked with the asterisk is in R-configuration. In certain embodiments -AB¹ and/or -AB² is of formula (i) and n is 17 and the stereocenter marked with the asterisk is in R-configuration. In certain embodiments -AB¹ and/or -AB² is of formula (i) and n is 18 and the stereocenter marked with the asterisk is in R-configuration. In certain embodiments -AB¹ and/or -AB² is of formula (i) and n is 19 and the stereocenter marked with the asterisk is in R-configuration. In certain embodiments -AB¹ and/or -AB² is of formula (i) and n is 20 and the stereocenter marked with the asterisk is in R-configuration. In certain embodiments -AB¹ and/or -AB² is of formula (i) and n is 21 and the stereocenter marked with the asterisk is in R-configuration. In certain embodiments -AB¹ and/or -AB² is of formula (i) and n is 22 and the stereocenter marked with the asterisk is in R-configuration.

In certain embodiments -AB¹ and/or -AB² is of formula (i) and n is 14 and the stereocenter marked with the asterisk is in S-configuration. In certain embodiments -AB¹ and/or -AB² is of formula (i) and n is 15 and the stereocenter marked with the asterisk is in S-configuration. In certain embodiments -AB¹ and/or -AB² is of formula (i) and n is 16 and the stereocenter marked with the asterisk is in S-configuration. In certain embodiments -AB¹ and/or -AB² is of formula (i) and n is 17 and the stereocenter marked with the asterisk is in S-configuration. In certain embodiments -AB¹ and/or -AB² is of formula (i) and n is 18 and the stereocenter marked with the asterisk is in S-configuration. In certain embodiments -AB¹ and/or -AB² is of formula (i) and n is 19 and the stereocenter marked with the asterisk is in S-configuration. In certain embodiments -AB¹ and/or -AB² is of formula (i) and n is 20 and the stereocenter marked with the asterisk is in S-configuration. In certain embodiments -AB¹ and/or -AB² is of formula (i) and n is 21 and the stereocenter marked with the asterisk is in S-configuration. In certain embodiments -AB¹ and/or -AB² is of formula (i) and n is 22 and the stereocenter marked with the asterisk is in S-configuration.

In certain embodiments -AB¹ and/or -AB² is of formula (i-a):

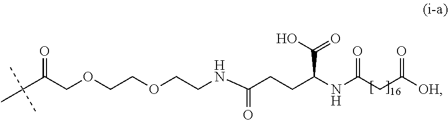

wherein
the dashed line indicates attachment to -L²- or -D-, respectively.

In certain embodiments -AB¹ and/or -AB² is of formula (i-b):

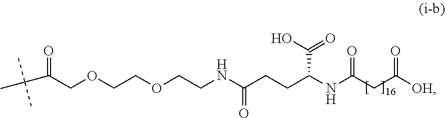

wherein
the dashed line indicates attachment to -L²- or -D-, respectively.

In certain embodiments -AB¹ and/or -AB² is of formula (i-c):

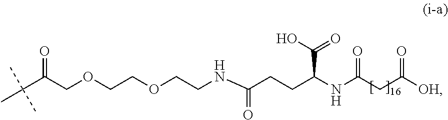

wherein
the dashed line indicates attachment to -L²- or -D-, respectively.

In certain embodiments -AB¹ and/or -AB² is of formula (i-d):

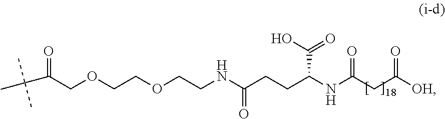

wherein
the dashed line indicates attachment to -L²- or -D-, respectively.

In certain embodiments -AB¹ and/or -AB² is of formula (i-e):

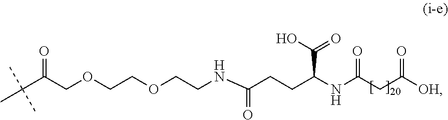

wherein
the dashed line indicates attachment to -L²- or -D-, respectively.

In certain embodiments -AB¹ and/or -AB² is of formula (i-f):

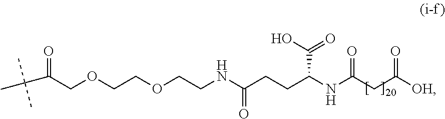

wherein
the dashed line indicates attachment to -L²- or -D-, respectively.

In certain embodiments -AB¹ and/or -AB² is of formula (ii)

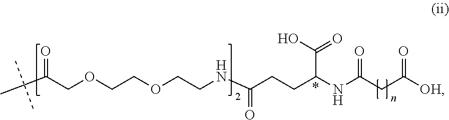

wherein the dashed line indicates attachment to -L²- or -D-, respectively;
n is an integer ranging from 14 to 22; and
the stereocenter marked with the asterisk is either in S- or R-configuration.

In certain embodiments -AB¹ and/or -AB² is of formula (ii) and n is 14. In certain embodiments—and/or or -AB² is of formula (ii) and n is 15. In certain embodiments -AB¹ and/or -AB² is of formula (ii) and n is 16. In certain embodiments -AB¹ and/or -AB² is of formula (ii) and n is 17. In certain embodiments -AB¹ and/or -AB² is of formula (ii) and n is 18. In certain embodiments -AB¹ and/or -AB² is of formula (ii) and n is 19. In certain embodiments -AB¹ and/or -AB² is of formula (ii) and n is 20. In certain embodiments -AB¹ and/or -AB² is of formula (ii) and n is 21. In certain embodiments -AB¹ and/or -AB² is of formula (ii) and n is 22.

In certain embodiments -AB¹ and/or -AB² is of formula (ii) and n is 14 and the stereocenter marked with the asterisk is in R-configuration. In certain embodiments -AB¹ and/or -AB² is of formula (ii) and n is 15 and the stereocenter marked with the asterisk is in R-configuration. In certain embodiments -AB¹ and/or -AB² is of formula (ii) and n is 16 and the stereocenter marked with the asterisk is in R-configuration. In certain embodiments -AB¹ and/or -AB² is of formula (ii) and n is 17 and the stereocenter marked with the asterisk is in R-configuration. In certain embodiments -AB¹ and/or -AB² is of formula (ii) and n is 18 and the stereocenter marked with the asterisk is in R-configuration. In certain embodiments -AB¹ and/or -AB² is of formula (ii) and n is 19 and the stereocenter marked with the asterisk is in R-configuration. In certain embodiments -AB¹ and/or -AB² is of formula (ii) and n is 20 and the stereocenter marked with the asterisk is in R-configuration. In certain embodiments -AB¹ and/or -AB² is of formula (ii) and n is 21 and the stereocenter marked with the asterisk is in R-configuration. In certain embodiments -AB¹ and/or -AB² is of formula (ii) and n is 22 and the stereocenter marked with the asterisk is in R-configuration.

In certain embodiments -AB¹ and/or -AB² is of formula (ii) and n is 14 and the stereocenter marked with the asterisk is in S-configuration. In certain embodiments -AB¹ and/or -AB² is of formula (ii) and n is 15 and the stereocenter marked with the asterisk is in S-configuration. In certain embodiments -AB¹ and/or -AB² is of formula (ii) and n is 16 and the stereocenter marked with the asterisk is in S-configuration. In certain embodiments -AB¹ and/or -AB² is of formula (ii) and n is 17 and the stereocenter marked with the asterisk is in S-configuration. In certain embodiments -AB¹ and/or -AB² is of formula (ii) and n is 18 and the stereocenter marked with the asterisk is in S-configuration. In certain embodiments -AB¹ and/or -AB² is of formula (ii) and n is 19 and the stereocenter marked with the asterisk is in S-configuration. In certain embodiments -AB¹ and/or -AB² is of formula (ii) and n is 20 and the stereocenter marked with the asterisk is in S-configuration. In certain embodiments -AB¹ and/or -AB² is of formula (ii) and n is 21 and the stereocenter marked with the asterisk is in S-configuration. In certain embodiments -AB¹ and/or -AB² is of formula (ii) and n is 22 and the stereocenter marked with the asterisk is in S-configuration.

In certain embodiments -AB¹ and/or -AB² is of formula (ii-a):

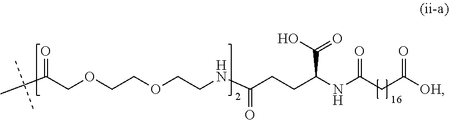

wherein
the dashed line indicates attachment to -L²- or -D-, respectively.

In certain embodiments -AB¹ and/or -AB² is of formula (ii-b):

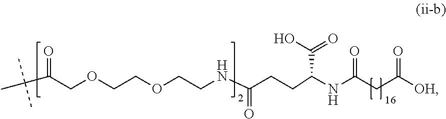

wherein
the dashed line indicates attachment to -L²- or -D-, respectively.

In certain embodiments -AB¹ and/or -AB² is of formula (ii-c):

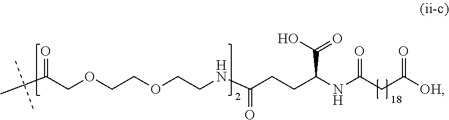

wherein
the dashed line indicates attachment to -L²- or -D-, respectively.

In certain embodiments -AB¹ and/or -AB² is of formula (ii-d):

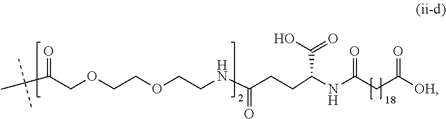

wherein
the dashed line indicates attachment to -L²- or -D-, respectively.

In certain embodiments -AB¹ and/or -AB² is of formula (ii-e):

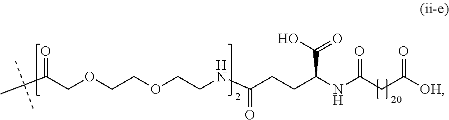

wherein
the dashed line indicates attachment to -L²- or -D-, respectively.

In certain embodiments -AB¹ and/or -AB² is of formula (ii-f):

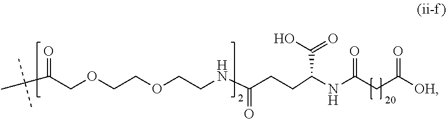

wherein
the dashed line indicates attachment to -L²- or -D-, respectively.

In certain embodiments -AB¹ and/or -AB² is of formula (iii)

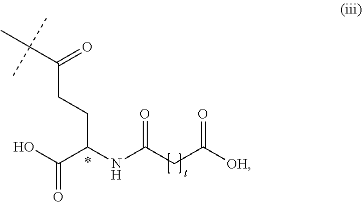

wherein
the dashed line indicates attachment to -L²- or -D-, respectively;
t is an integer ranging from and including 14 to 22; and
the stereocenter marked with the asterisk is either in S- or R-configuration.

In certain embodiments -AB¹ and/or -AB² is of formula (iii) and t is 14. In certain embodiments -AB¹ and/or -AB² is of formula (iii) and t is 15. In certain embodiments -AB¹ and/or -AB² is of formula (iii) and t is 16. In certain embodiments -AB¹ and/or -AB² is of formula (iii) and t is 17. In certain embodiments -AB¹ and/or -AB² is of formula (iii) and t is 18. In certain embodiments -AB¹ and/or -AB² is of formula (iii) and t is 19. In certain embodiments -AB¹ and/or -AB² is of formula (iii) and t is 20. In certain embodiments -AB¹ and/or -AB² is of formula (iii) and t is 21. In certain embodiments -AB¹ and/or -AB² is of formula (iii) and t is 22.

In certain embodiments -AB¹ and/or -AB² is of formula (iii) and t is 14 and the stereocenter marked with the asterisk is in R-configuration. In certain embodiments -AB¹ and/or -AB² is of formula (iii) and t is 15 and the stereocenter marked with the asterisk is in R-configuration. In certain embodiments -AB¹ and/or -AB² is of formula (iii) and t is 16 and the stereocenter marked with the asterisk is in R-configuration. In certain embodiments -AB¹ and/or -AB² is of formula (iii) and t is 17 and the stereocenter marked with the asterisk is in R-configuration. In certain embodiments -AB¹ and/or -AB² is of formula (iii) and t is 18 and the stereocenter marked with the asterisk is in R-configuration. In certain embodiments -AB¹ and/or -AB² is of formula (iii) and t is 19 and the stereocenter marked with the asterisk is in R-configuration. In certain embodiments -AB¹ and/or -AB² is of formula (iii) and t is 20 and the stereocenter marked with the asterisk is in R-configuration. In certain embodiments -AB¹ and/or -AB² is of formula (iii) and t is 21 and the stereocenter marked with the asterisk is in R-configuration. In certain embodiments -AB¹ and/or -AB² is of formula (iii) and t is 22 and the stereocenter marked with the asterisk is in R-configuration.

In certain embodiments -AB¹ and/or -AB² is of formula (iii) and t is 14 and the stereocenter marked with the asterisk is in S-configuration. In certain embodiments -AB¹ and/or -AB² is of formula (iii) and t is 15 and the stereocenter marked with the asterisk is in S-configuration. In certain embodiments -AB¹ and/or -AB² is of formula (iii) and t is 16 and the stereocenter marked with the asterisk is in S-configuration. In certain embodiments -AB¹ and/or -AB² is of formula (iii) and t is 17 and the stereocenter marked with the asterisk is in S-configuration. In certain embodiments -AB¹ and/or -AB² is of formula (iii) and t is 18 and the stereocenter marked with the asterisk is in S-configuration. In certain embodiments -AB¹ and/or -AB² is of formula (iii) and t is 19 and the stereocenter marked with the asterisk is in S-configuration. In certain embodiments -AB¹ and/or -AB² is of formula (iii) and t is 20 and the stereocenter marked with the asterisk is in S-configuration. In certain embodiments -AB¹ and/or -AB² is of formula (iii) and t is 21 and the stereocenter marked with the asterisk is in S-configuration. In certain embodiments -AB¹ and/or -AB² is of formula (iii) and t is 22 and the stereocenter marked with the asterisk is in S-configuration.

In certain embodiments -AB¹ and/or -AB² is of formula (iii-a)

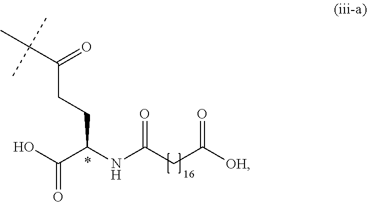

wherein
the dashed line indicates attachment to -L²- or -D-, respectively.

In certain embodiments -AB¹ and/or -AB² is of formula (iii-b)

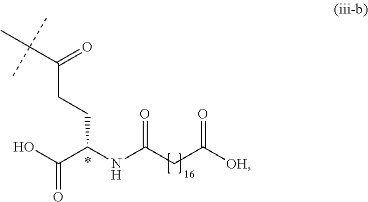

wherein
the dashed line indicates attachment to -L²- or -D-, respectively;

In certain embodiments -AB¹ and/or -AB² is of formula (iii-c)

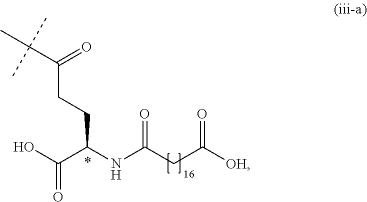

wherein
the dashed line indicates attachment to -L²- or -D-, respectively.

In certain embodiments -AB¹ and/or -AB² is of formula (iii-d)

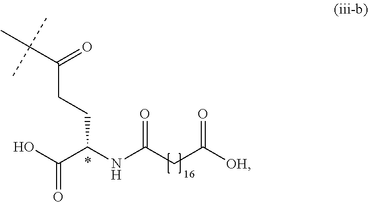

wherein
the dashed line indicates attachment to -L²- or -D-, respectively.

In certain embodiments -AB¹ and/or -AB² is of formula (iii-e)

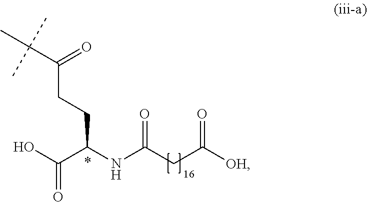

wherein
the dashed line indicates attachment to -L²- or -D-, respectively.

In certain embodiments -AB¹ and/or -AB² is of formula (iii-f)

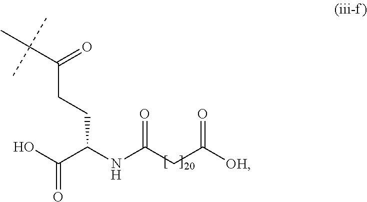

wherein
the dashed line indicates attachment to -L²- or -D-, respectively.

In certain embodiments -AB¹ and/or -AB² is of formula (iv)

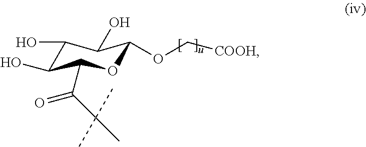

wherein
the dashed line indicates attachment to -L²- or -D-, respectively, and
u is an integer ranging from and including 14 to 22.

In certain embodiments -AB¹ and/or -AB² is of formula (iv) and u is 14. In certain embodiments -AB¹ and/or -AB² is of formula (iv) and u is 15. In certain embodiments -AB¹ and/or -AB² is of formula (iv) and u is 16. In certain embodiments -AB¹ and/or -AB² is of formula (iv) and u is 17. In certain embodiments -AB¹ and/or -AB² is of formula (iv) and u is 18. In certain embodiments -AB¹ and/or -AB² is of formula (iv) and u is 19. In certain embodiments -AB¹ and/or -AB² is of formula (iv) and u is 20. In certain embodiments -AB¹ and/or -AB² is of formula (iv) and u is 21. In certain embodiments -AB¹ and/or -AB² is of formula (iv) and u is 22.

In certain embodiments -AB¹ and/or -AB² is of formula (v)

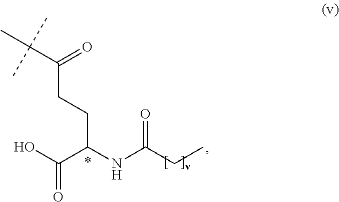

wherein
the dashed line indicates attachment to -L²- or -D-, respectively;
v is an integer ranging from and including 14 to 22; and
the stereocenter marked with the asterisk is either in S- or R-configuration.

In certain embodiments -AB¹ and/or -AB² is of formula (v) and v is 14. In certain embodiments -AB¹ and/or -AB² is of formula (v) and v is 15. In certain embodiments -AB¹ and/or -AB² is of formula (v) and v is 16. In certain embodiments -AB¹ and/or -AB² is of formula (v) and v is 17. In certain embodiments -AB¹ and/or -AB² is of formula (v) and v is 18. In certain embodiments -AB¹ and/or -AB² is of formula (v) and v is 19. In certain embodiments -AB¹ and/or -AB² is of formula (v) and v is 20. In certain embodiments -AB¹ and/or -AB² is of formula (v) and v is 21. In certain embodiments -AB¹ and/or -AB² is of formula (v) and v is 22.

In certain embodiments -AB¹ and/or -AB² is of formula (v) and v is 14 and the stereocenter marked with the asterisk is in R-configuration. In certain embodiments -AB¹ and/or -AB² is of formula (v) and v is 15 and the stereocenter marked with the asterisk is in R-configuration. In certain embodiments -AB¹ and/or -AB² is of formula (v) and v is 16 and the stereocenter marked with the asterisk is in R-configuration. In certain embodiments -AB¹ and/or -AB² is of formula (v) and v is 17 and the stereocenter marked with the asterisk is in R-configuration. In certain embodiments -AB¹ and/or -AB² is of formula (v) and v is 18 and the stereocenter marked with the asterisk is in R-configuration. In certain embodiments -AB¹ and/or -AB² is of formula (v) and v is 19 and the stereocenter marked with the asterisk is in R-configuration. In certain embodiments -AB¹ and/or -AB² is of formula (v) and v is 20 and the stereocenter marked with the asterisk is in R-configuration. In certain embodiments -AB¹ and/or -AB² is of formula (v) and v is 21 and the stereocenter marked with the asterisk is in R-configuration. In certain embodiments -AB¹ and/or -AB² is of formula (v) and v is 22 and the stereocenter marked with the asterisk is in R-configuration.

In certain embodiments -AB¹ and/or -AB² is of formula (v) and v is 14 and the stereocenter marked with the asterisk is in S-configuration. In certain embodiments -AB¹ and/or -AB² is of formula (v) and v is 15 and the stereocenter marked with the asterisk is in S-configuration. In certain embodiments -AB¹ and/or -AB² is of formula (v) and v is 16 and the stereocenter marked with the asterisk is in S-configuration. In certain embodiments -AB¹ and/or -AB² is of formula (v) and v is 17 and the stereocenter marked with the asterisk is in S-configuration. In certain embodiments -AB¹ and/or -AB² is of formula (v) and v is 18 and the stereocenter marked with the asterisk is in S-configuration. In certain embodiments -AB¹ and/or -AB² is of formula (v) and v is 19 and the stereocenter marked with the asterisk is in S-configuration. In certain embodiments -AB¹ and/or -AB² is of formula (v) and v is 20 and the stereocenter marked with the asterisk is in S-configuration. In certain embodiments -AB¹ and/or -AB² is of formula (v) and v is 21 and the stereocenter marked with the asterisk is in S-configuration. In certain embodiments -AB¹ and/or -AB² is of formula (v) and v is 22 and the stereocenter marked with the asterisk is in S-configuration.

In certain embodiments -AB¹ and/or -AB² is of formula (v-a)

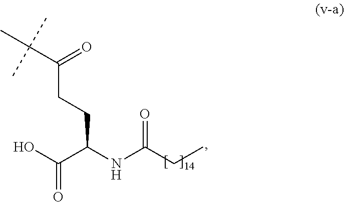

wherein
the dashed line indicates attachment to -L²- or -D-, respectively.

In certain embodiments -AB¹ and/or -AB² is of formula (v-b)

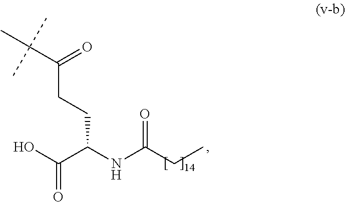

wherein
the dashed line indicates attachment to -L²- or -D-, respectively;

In certain embodiments -AB¹ and/or -AB² is of formula (v-c)

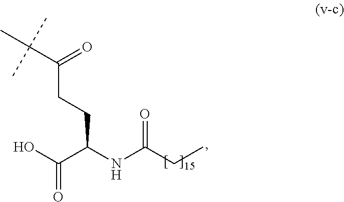

wherein
the dashed line indicates attachment to -L²- or -D-, respectively.

In certain embodiments -AB¹ and/or -AB² is of formula (v-d)

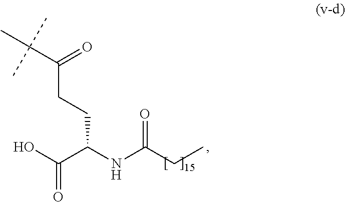

wherein
the dashed line indicates attachment to -L²- or -D-, respectively.

In certain embodiments -AB¹ and/or -AB² is of formula (vi-e)

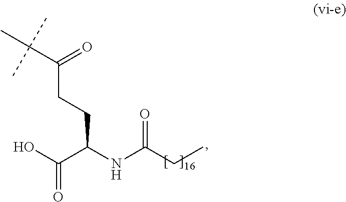

wherein
the dashed line indicates attachment to -L²- or -D-, respectively.

In certain embodiments -AB¹ and/or -AB² is of formula (vi-f)

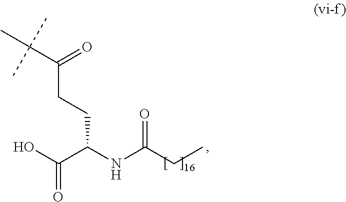

wherein
the dashed line indicates attachment to -L²- or -D-, respectively.

In certain embodiments -AB¹ and/or -AB² is of formula (vii)

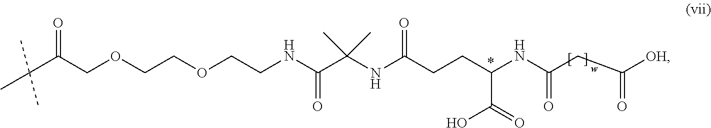

wherein the dashed line indicates attachment to -L²- or -D-, respectively;
w is an integer ranging from and including 14 to 22; and
the stereocenter marked with the asterisk is either in S- or R-configuration.

In certain embodiments -AB¹ and/or -AB² is of formula (vii) and w is 14. In certain embodiments -AB¹ and/or -AB² is of formula (vii) and w is 15. In certain embodiments -AB¹ and/or -AB² is of formula (vii) and w is 16. In certain embodiments -AB¹ and/or -AB² is of formula (vii) and w is 17. In certain embodiments -AB¹ and/or -AB² is of formula (vii) and w is 18. In certain embodiments -AB¹ and/or -AB² is of formula (vii) and w is 19. In certain embodiments -AB¹ and/or -AB² is of formula (vii) and w is 20. In certain embodiments -AB¹ and/or -AB² is of formula (vii) and w is 21. In certain embodiments -AB¹ and/or -AB² is of formula (vii) and w is 22.

In certain embodiments -AB¹ and/or -AB² is of formula (vii) and w is 14 and the stereocenter marked with the asterisk is in R-configuration. In certain embodiments -AB¹ and/or -AB² is of formula (vii) and w is 15 and the stereocenter marked with the asterisk is in R-configuration. In certain embodiments -AB¹ and/or -AB² is of formula (vii) and w is 16 and the stereocenter marked with the asterisk is in R-configuration. In certain embodiments -AB¹ and/or -AB² is of formula (vii) and w is 17 and the stereocenter marked with the asterisk is in R-configuration. In certain embodiments -AB¹ and/or -AB² is of formula (vii) and w is 18 and the stereocenter marked with the asterisk is in R-configuration. In certain embodiments -AB¹ and/or -AB² is of formula (vii) and w is 19 and the stereocenter marked with the asterisk is in R-configuration.

In certain embodiments -AB¹ and/or -AB² is of formula (vii) and w is 20 and the stereocenter marked with the asterisk is in R-configuration. In certain embodiments -AB¹ and/or -AB² is of formula (vii) and w is 21 and the stereocenter marked with the asterisk is in R-configuration. In certain embodiments -AB¹ and/or -AB² is of formula (vii) and w is 22 and the stereocenter marked with the asterisk is in R-configuration.

In certain embodiments -AB¹ and/or -AB² is of formula (vii) and w is 14 and the stereocenter marked with the asterisk is in S-configuration. In certain embodiments -AB¹ and/or -AB² is of formula (vii) and w is 15 and the stereocenter marked with the asterisk is in S-configuration. In certain embodiments -AB¹ and/or -AB² is of formula (vii) and w is 16 and the stereocenter marked with the asterisk is in S-configuration. In certain embodiments -AB¹ and/or -AB² is of formula (vii) and w is 17 and the stereocenter marked with the asterisk is in S-configuration. In certain embodiments -AB¹ and/or -AB² is of formula (vii) and w is 18 and the stereocenter marked with the asterisk is in S-configuration. In certain embodiments -AB¹ and/or -AB² is of formula (vii) and w is 19 and the stereocenter marked with the asterisk is in S-configuration. In certain embodiments -AB¹ and/or -AB² is of formula (vii) and w is 20 and the stereocenter marked with the asterisk is in S-configuration. In certain embodiments -AB¹ and/or -AB² is of formula (vii) and w is 21 and the stereocenter marked with the asterisk is in S-configuration. In certain embodiments -AB¹ and/or -AB² is of formula (vii) and w is 22 and the stereocenter marked with the asterisk is in S-configuration.

In certain embodiments -AB¹ and/or -AB² is of formula (vii-a):

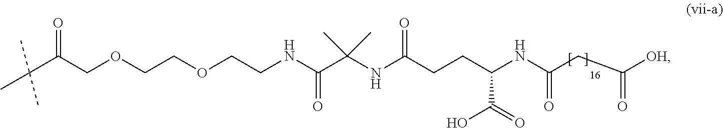

wherein
the dashed line indicates attachment to -L²- or -D-, respectively.

In certain embodiments -AB¹ and/or -AB² is of formula (vii-b):

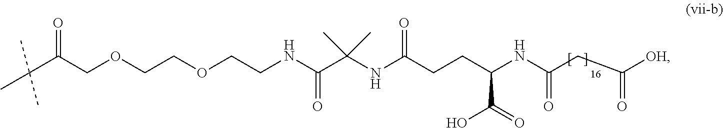

wherein
the dashed line indicates attachment to -L²- or -D-, respectively.

In certain embodiments -AB¹ and/or -AB² is of formula (vii-c):

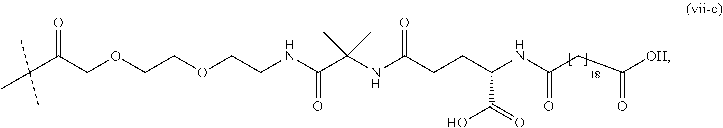

wherein
the dashed line indicates attachment to -L²- or -D-, respectively.

In certain embodiments -AB¹ and/or -AB² is of formula (vii-d):

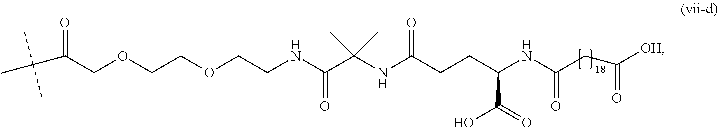

wherein
the dashed line indicates attachment to -L²- or -D-, respectively.

In certain embodiments -AB¹ and/or -AB² is of formula (vii-e):

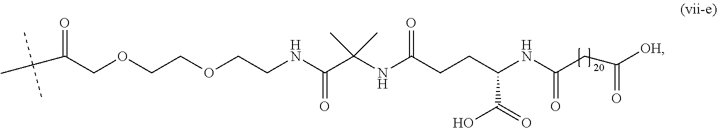

wherein
the dashed line indicates attachment to -L²- or -D-, respectively.

In certain embodiments -AB¹ and/or -AB² is of formula (vii-f):

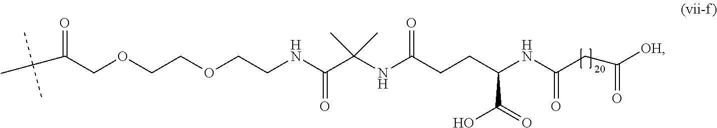

wherein
the dashed line indicates attachment to -L²- or -D-, respectively.

In certain embodiments -AB¹ and/or -AB² is of formula (viii)

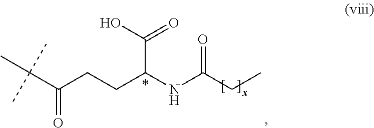

wherein the dashed line indicates attachment to -L²- or -D-, respectively;
w is an integer ranging from and including 14 to 22; and
the stereocenter marked with the asterisk is either in S- or R-configuration.

In certain embodiments -AB¹ and/or -AB² is of formula (viii) and x is 14. In certain embodiments -AB¹ and/or -AB² is of formula (viii) and x is 15. In certain embodiments -AB¹ and/or -AB² is of formula (viii) and x is 16. In certain embodiments -AB¹ and/or -AB² is of formula (viii) and x is 17. In certain embodiments -AB¹ and/or -AB² is of formula (viii) and x is 18. In certain embodiments -AB¹ and/or -AB² is of formula (viii) and x is 19. In certain embodiments -AB¹ and/or -AB² is of formula (viii) and x is 20. In certain embodiments -AB¹ and/or -AB² is of formula (viii) and x is 21. In certain embodiments -AB¹ and/or -AB² is of formula (viii) and x is 22.

In certain embodiments -AB¹ and/or -AB² is of formula (viii) and x is 14 and the stereocenter marked with the asterisk is in R-configuration. In certain embodiments -AB¹ and/or -AB² is of formula (viii) and x is 15 and the stereocenter marked with the asterisk is in R-configuration. In certain embodiments -AB¹ and/or -AB² is of formula (viii) and x is 16 and the stereocenter marked with the asterisk is in R-configuration. In certain embodiments -AB¹ and/or -AB² is of formula (viii) and x is 17 and the stereocenter marked with the asterisk is in R-configuration. In certain embodiments -AB¹ and/or -AB² is of formula (viii) and x is 18 and the stereocenter marked with the asterisk is in R-configuration. In certain embodiments -AB¹ and/or -AB² is of formula (viii) and x is 19 and the stereocenter marked with the asterisk is in R-configuration. In certain embodiments -AB¹ and/or -AB² is of formula (viii) and x is 20 and the stereocenter marked with the asterisk is in R-configuration. In certain embodiments -AB¹ and/or -AB² is of formula (viii) and x is 21 and the stereocenter marked with the asterisk is in R-configuration. In certain embodiments -AB¹ and/or -AB² is of formula (viii) and x is 22 and the stereocenter marked with the asterisk is in R-configuration.

In certain embodiments -AB¹ and/or -AB² is of formula (viii) and x is 14 and the stereocenter marked with the asterisk is in S-configuration. In certain embodiments -AB¹ and/or -AB² is of formula (viii) and x is 15 and the stereocenter marked with the asterisk is in S-configuration. In certain embodiments -AB¹ and/or -AB² is of formula (viii) and x is 16 and the stereocenter marked with the asterisk is in S-configuration. In certain embodiments -AB¹ and/or -AB² is of formula (viii) and x is 17 and the stereocenter marked with the asterisk is in S-configuration. In certain embodiments -AB¹ and/or -AB² is of formula (viii) and x is 18 and the stereocenter marked with the asterisk is in S-configuration. In certain embodiments -AB¹ and/or -AB² is of formula (viii) and x is 19 and the stereocenter marked with the asterisk is in S-configuration. In certain embodiments -AB¹ and/or -AB² is of formula (viii) and x is 20 and the stereocenter marked with the asterisk is in S-configuration. In certain embodiments -AB¹ and/or -AB² is of formula (viii) and x is 21 and the stereocenter marked with the asterisk is in S-configuration. In certain embodiments -AB¹ and/or -AB² is of formula (viii) and x is 22 and the stereocenter marked with the asterisk is in S-configuration.

In Certain Embodiments -AB¹ and/or -AB² is of Formula (viii-a)

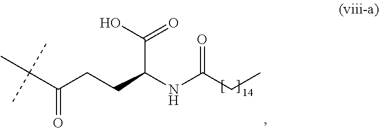

wherein the dashed line indicates attachment to -L²- or -D-, respectively.
   In Certain Embodiments -AB¹ and/or -AB² is of Formula (viii-b)

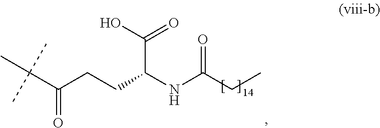

wherein the dashed line indicates attachment to -L²- or -D-, respectively.
   In Certain Embodiments -AB¹ and/or -AB² is of Formula (viii-c)

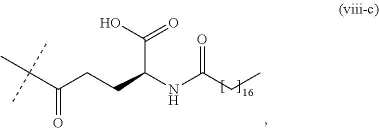

wherein the dashed line indicates attachment to -L²- or -D-, respectively.
   In Certain Embodiments -AB¹ and/or -AB² is of Formula (viii-d)

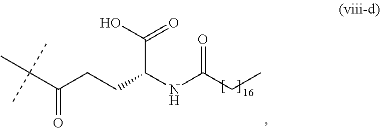

wherein the dashed line indicates attachment to -L²- or -D-, respectively.
   In Certain Embodiments -AB¹ and/or -AB² is of Formula (viii-e)

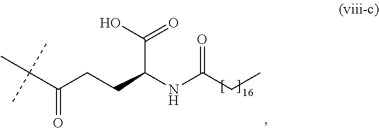

wherein the dashed line indicates attachment to -L²- or -D-, respectively.
   In Certain Embodiments -AB¹ and/or -AB² is of Formula (viii-f)

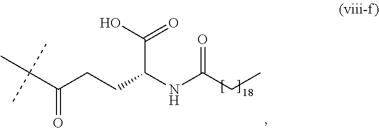

wherein the dashed line indicates attachment to -L²- or -D-, respectively.
   In Certain Embodiments -AB¹ and/or -AB² is of Formula (ix-i)

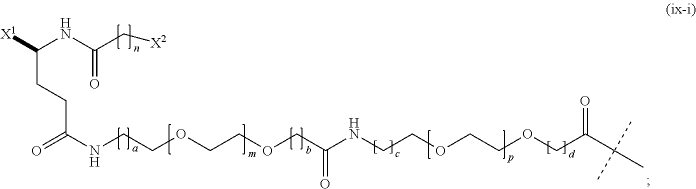

wherein
X¹—is

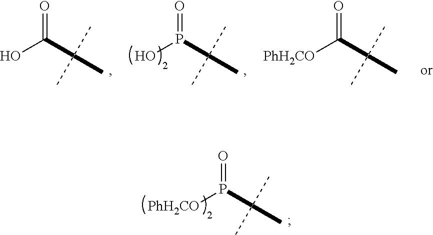

X² is

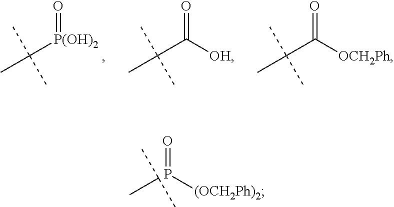

with Ph being phenyl,
a is 0, 1, 2, 3, 4, 5, 6, 7, 8, 9 or 10;
b is 1, 2, 3, 4, 5, 6, 7, 8, 9 or 10;
c is 0, 1, 2, 3, 4, 5, 6, 7, 8, 9 or 10;
d is 1, 2, 3, 4, 5, 6, 7, 8, 9 or 10;
m is 1, 2, 3, 4, 5, 6, 7, 8, 9 or 10;
n is 10, 11, 12, 13, 14, 15, 16, 17, 18, 19, 20, 21, 22, 23 or 24;
p is 1, 2, 3, 4, 5, 6, 7, 8, 9 or 10.

In certain embodiments -AB¹ and/or -AB² is of formula (ix-i) with a being 1, b being 1, c being 1, d being 1, m being 1, n being 18 and m being 1.

In certain embodiments -AB¹ and/or -AB² is of formula (ix-i) and X¹—is

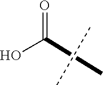

### and —X² is

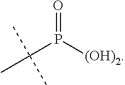

In certain embodiments -AB¹ and/or -AB² is of formula (ix-i) and X¹—is

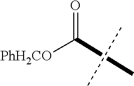

### and —X² is

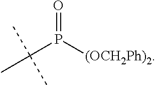

In certain embodiments -AB¹ and/or -AB² is of formula (ix-i) and X¹—is

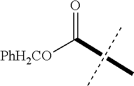

### and —X² is

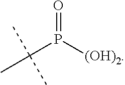

In certain embodiments -AB¹ and/or -AB² is of formula (ix-i) and X¹—is

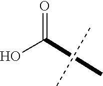

### and —X² is

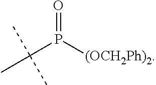

In certain embodiments -AB¹ and/or -AB² is of formula (ix-i) and X¹—is

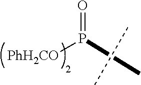

### and —X² is

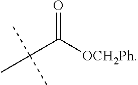

In certain embodiments -AB¹ and/or -AB² is of formula (ix-i) and X¹—is

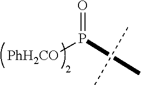

### —X² is

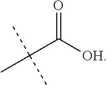

In certain embodiments -AB¹ and/or -AB² is of formula (ix-i) and X¹- is

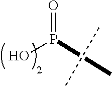

### and —X² is

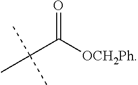

In certain embodiments -AB¹ and/or -AB² is of formula (ix-i) and X¹—is

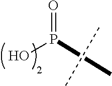

### and —X² is

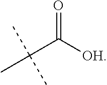

In certain embodiments -AB¹ and/or -AB² is of formula (ix-i) and X¹—is

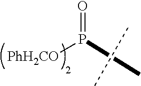

### and —X² is

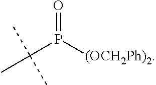

In certain embodiments -AB¹ and/or -AB² is of formula (ix-i) and X¹—is

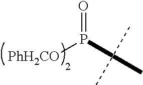

### and —X² is

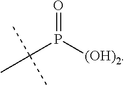

In certain embodiments -AB¹ and/or -AB² is of formula (ix-i) and X¹—is

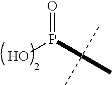

### and —X² is

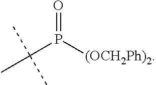

In certain embodiments -AB¹ and/or -AB² is of formula (ix-i) and X¹—is

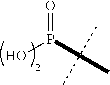

### and —X² is

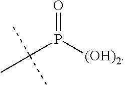

In certain embodiments -AB¹ and/or -AB² is a peptidic albumin-binding moiety.

If -D- is a peptide or protein drug moiety, a peptidic moiety -AB² may be fused to the N- or C-terminus of -D-, either directly or with a peptidic spacer between -D- and -AB².

In certain embodiments -AB¹ and/or -AB² is selected from the group consisting of

(SEQ ID NO: 34)
WWEQDRDWDFDVFGGGTP;
(SEQ ID NO: 35)
DICLPRWGCLW,

wherein the cysteines at position 3 and 9 are connected via a disulfide bridge;

(SEQ ID NO: 36)
RLIEDICLPRWGCLWEDD,

wherein the cysteines at position 7 and 13 are connected via a disulfide bridge;

(SEQ ID NO: 37)
LAEAKVLANRELDKYGVSDFYKRLINKAKTVEGVEALKLHILAALP;
(SEQ ID NO: 38)
IAEAKEAANAELDSYGVSDFYKRLIDKAKTVEGVEALKDAILAALP;
and
(SEQ ID NO: 39)
X_{I}EYEX₂EYE,

wherein X₁ is fluorescein-(AEEA), X₂ is K(palmitate), and AEEA is 2-(2-(2-aminoethoxy)acetyl.

In certain embodiments -AB¹ and/or -AB² is of SEQ ID NO:34. In certain embodiments -AB¹ and/or -AB² is of SEQ ID NO:35. In certain embodiments -AB¹ and/or -AB² is of SEQ ID NO:36. In certain embodiments -AB¹ and/or -AB² is of SEQ ID NO:37. In certain embodiments -AB¹ and/or -AB² is of SEQ ID NO:38. In certain embodiments -AB¹ and/or -AB² is of SEQ ID NO:39.

In certain embodiments -AB¹ is of formula (XIX)

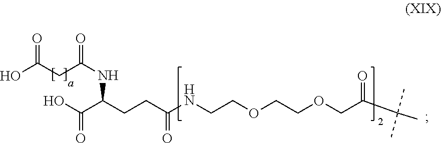

wherein
the dashed line indicates attachment to -L²-; and
a is 14, 15, 16, 17, 18, 19, 20, 21 or 22.

In certain embodiments a of formula (XIX) is 18.

In certain embodiments -AB¹ and/or -AB² is of formula (A-a):

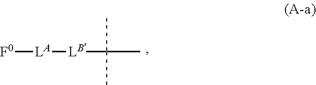

wherein
the dashed line indicates attachment to -L²- or -D-, respectively;
—F⁰ and -L^{A}- are used as defined in formula (A),
-L^{B′}- is a polymeric moiety.

The moiety -L^{B′}- is a polymeric moiety, meaning that it comprises at least one polymer moiety. In certain embodiments the one or more polymer moiety has a molecular weight of at least 450 Da. In certain embodiments the one or more polymer moiety has a molecular weight of at least 1 kDa. In certain embodiments the one or more polymer moiety has a molecular weight of at least 1.5 kDa. In certain embodiments the one or more polymer moiety has a molecular weight of at least 2 kDa. In certain embodiments the one or more polymer moiety has a molecular weight of at least 2.5 kDa. In certain embodiments the one or more polymer moiety has a molecular weight of at least 3 kDa. In certain embodiments the one or more polymer moiety has a molecular weight of at least 3.5 kDa. In certain embodiments the one or more polymer moiety has a molecular weight of at least 4 kDa. In certain embodiments the one or more polymer moiety has a molecular weight of at least 5 kDa. In certain embodiments the one or more polymer moiety has a maximum molecular weight of 160 kDa. In certain embodiments the one or more polymer moiety has a maximum molecular weight of 120 kDa. In certain embodiments the one or more polymer moiety has a maximum molecular weight of 100 kDa. In certain embodiments the one or more polymer moiety has a maximum molecular weight of 80 kDa. In certain embodiments the one or more polymer moiety has a maximum molecular weight of 70 kDa. In certain embodiments the one or more polymer moiety has a maximum molecular weight of 60 kDa. In certain embodiments the one or more polymer moiety has a maximum molecular weight of 50 kDa. In certain embodiments the one or more polymer moiety has a maximum molecular weight of 40 kDa. In certain embodiments the one or more polymer moiety has a molecular weight of about 450 Da. In certain embodiments the one or more polymer moiety has a molecular weight of about 1 kDa. In certain embodiments the one or more polymer moiety has a molecular weight of about 1.5 kDa. In certain embodiments the one or more polymer moiety has a molecular weight of about 2 kDa. In certain embodiments the one or more polymer moiety has a molecular weight of about 2.5 kDa. In certain embodiments the one or more polymer moiety has a molecular weight of about 3 kDa. In certain embodiments the one or more polymer moiety has a molecular weight of about 3.5 kDa. In certain embodiments the one or more polymer moiety has a molecular weight of about 4 kDa. In certain embodiments the one or more polymer moiety has a molecular weight of about 4.5 kDa. In certain embodiments the one or more polymer moiety has a molecular weight of about 5 kDa. In certain embodiments the one or more polymer moiety has a molecular weight of about 5.5 kDa. In certain embodiments the one or more polymer moiety has a molecular weight of about 6 kDa. In certain embodiments the one or more polymer moiety has a molecular weight of about 6.5 kDa. In certain embodiments the one or more polymer moiety has a molecular weight of about 7 kDa. In certain embodiments the one or more polymer moiety has a molecular weight of about 7.5 kDa. In certain embodiments the one or more polymer moiety has a molecular weight of about 8 kDa. In certain embodiments the one or more polymer moiety has a molecular weight of about 8.5 kDa. In certain embodiments the one or more polymer moiety has a molecular weight of about 9 kDa. In certain embodiments the one or more polymer moiety has a molecular weight of about 9.5 kDa. In certain embodiments the one or more polymer moiety has a molecular weight of about 10 kDa. It is understood that if -L^{B′}- comprises one polymer moiety the minimum and maximum molecular weights provided above apply to this one polymer moiety and if -L^{B′}- comprises more than one polymer moiety the minimum and maximum molecular weights provided above refer to the minimum and maximum molecular weight of all polymer moieties together.

In certain embodiments the one or more polymer moiety of -L^{B′}- has a Flory radius of at least 1.2 nm. In certain embodiments the one or more polymer moiety of -L^{B′}- has a Flory radius of at least 1.5 nm. In certain embodiments the one or more polymer moiety of -L^{B′}- has a Flory radius of at least 2 nm. In certain embodiments the one or more polymer moiety of -L^{B′}- has a Flory radius of at least 2.5 nm. In certain embodiments the one or more polymer moiety of -L^{B′}- has a Flory radius of at least 3 nm. In certain embodiments the one or more polymer moiety of -L^{B′}- has a Flory radius of at least 3.5 nm. In certain embodiments the one or more polymer moiety of -L^{B′}- has a Flory radius of at least 4 nm. In certain embodiments the one or more polymer moiety of -L^{B′}- has a Flory radius of at least 4.5 nm. In certain embodiments the one or more polymer moiety of -L^{B′}- has a Flory radius of at least 5 nm. In certain embodiments the one or more polymer moiety of -L^{B′}- has a Flory radius of no more than 200 nm. In certain embodiments the one or more polymer moiety of -L^{B′}- has a Flory radius of no more than 175 nm. In certain embodiments the one or more polymer moiety of -L^{B′}- has a Flory radius of no more than 150 nm. In certain embodiments the one or more polymer moiety of -L^{B′}- has a Flory radius of no more than 125 nm. In certain embodiments the one or more polymer moiety of -L^{B′}- has a Flory radius of no more than 100 nm. In certain embodiments the one or more polymer moiety of -L^{B′}- has a Flory radius of no more than 75 nm. In certain embodiments the one or more polymer moiety of -L^{B′}- has a Flory radius of no more than 50 nm. In certain embodiments the one or more polymer moiety of -L^{B′}- has a Flory radius of no more than 45 nm. In certain embodiments the one or more polymer moiety of -L^{B′}- has a Flory radius of no more than 40 nm. In certain embodiments the one or more polymer moiety of -L^{B′}- has a Flory radius of no more than 35 nm. In certain embodiments the one or more polymer moiety of -L^{B′}- has a Flory radius of no more than 30 nm. In certain embodiments the one or more polymer moiety of -L^{B′}- has a Flory radius of about 1.2 nm. In certain embodiments the one or more polymer moiety of -L^{B′}- has a Flory radius of about 1.5 nm. In certain embodiments the one or more polymer moiety of -L^{B′}- has a Flory radius of about 2 nm. In certain embodiments the one or more polymer moiety of -L^{B′}- has a Flory radius of about 2.5 nm. In certain embodiments the one or more polymer moiety of -L^{B′}- has a Flory radius of about 3 nm. In certain embodiments the one or more polymer moiety of -L^{B′}- has a Flory radius of about 3.5 nm. In certain embodiments the one or more polymer moiety of -L^{B′}- has a Flory radius of about 4 nm. In certain embodiments the one or more polymer moiety of -L^{B′}- has a Flory radius of about 4.5 nm. In certain embodiments the one or more polymer moiety of -L^{B′}- has a Flory radius of about 5 nm. In certain embodiments the one or more polymer moiety of -L^{B′}- has a Flory radius of about 5.5 nm. In certain embodiments the one or more polymer moiety of -L^{B′}- has a Flory radius of about 6 nm. In certain embodiments the one or more polymer moiety of -L^{B′}- has a Flory radius of about 6.5 nm. In certain embodiments the one or more polymer moiety of -L^{B′}- has a Flory radius of about 7 nm. In certain embodiments the one or more polymer moiety of -L^{B′}- has a Flory radius of about 8.5 nm. In certain embodiments the one or more polymer moiety of -L^{B′}- has a Flory radius of about 9 nm. In certain embodiments the one or more polymer moiety of -L^{B′}- has a Flory radius of about 10 nm. It is understood that if -L^{B′}- comprises one polymer moiety the Flory radius provided above applies to this one polymer moiety and if -L^{B′}- comprises more than one polymer moiety the Flory radius provided above refers to the Flory radius of all polymer moieties together.

-L^{B′}- comprises one or more polymer moiety, such as polymer moiety selected from the group consisting of poly(2-methacryloyl-oxyethyl phosphonyl cholines), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycol), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments -L^{B′}- comprises a PEG-based polymer. In certain embodiments -L^{B′}- comprises a hyaluronic acid-based polymer. In certain embodiments -L^{B′}- comprises a random coil polymer. In certain embodiments -L^{B′}- comprises a poly-sarcosine polymer.

In certain embodiments the distance between any two albumin-binding moieties of a compound is such that they can bind to two different binding sites on the same albumin molecule or to two different albumin molecules. All or some albumin binding moieties may bind to different albumin molecules and/or two or more albumin binding moieties may bind to one albumin molecule. In general, if a compound has x albumin binding moieties, they may bind to up to x albumin molecules. It is understood that not all albumin binding moieties of a compound may be bound to an albumin molecule at any given time.

If a compound is of formula (Ia) with x=1, the moiety -AB¹ may be bound to a first albumin molecule and the moiety -AB² may be bound to a second albumin molecule, which is different from the first albumin molecule. Alternatively, if a compound is of formula (Ia) with x=1, the moiety -AB¹ may be bound to a first binding site on a first albumin molecule and the moiety -AB² may be bound to a second binding site on the same albumin molecule, which second binding site is different from the first binding site.

Binding to two or more different albumin molecules increases the molecular weight of the compound significantly, which has an advantageous effect on circulation half-life.

In certain embodiments -L^{B′}- of formula (A-a) is of formula (a-3′)

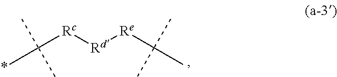

wherein
the unmarked dashed line indicates attachment to -L²- or -D-;
the dashed line marked with the asterisk indicates attachment to -LA;
—R^{c}—is

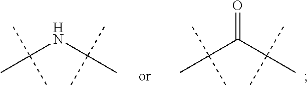

—R^{d′}—is a polymeric moiety; and
R^{e}—is selected from the group consisting of —CH₂—,

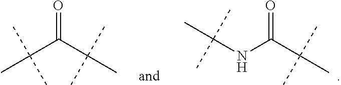

If —R^{b}—of formula (A-a) is

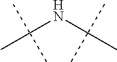

then —R^{c}—of formula (a-3′) is

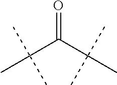

and if —R^{b}—of formula (A-a) is

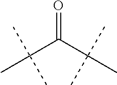

then —R^{c}—of formula (a-3′) is

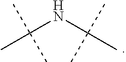

The moiety —R^{d′}—of formula (a-3′) is a polymeric moiety, meaning that it comprises at least one polymer moiety. In certain embodiments the one or more polymer moiety has a molecular weight of at least 450 Da. In certain embodiments the one or more polymer moiety has a molecular weight of at least 1 kDa. In certain embodiments the one or more polymer moiety has a molecular weight of at least 1.5 kDa. In certain embodiments the one or more polymer moiety has a molecular weight of at least 2 kDa. In certain embodiments the one or more polymer moiety has a molecular weight of at least 2.5 kDa. In certain embodiments the one or more polymer moiety has a molecular weight of at least 3 kDa. In certain embodiments the one or more polymer moiety has a molecular weight of at least 3.5 kDa. In certain embodiments the one or more polymer moiety has a molecular weight of at least 4 kDa. In certain embodiments the one or more polymer moiety has a molecular weight of at least 5 kDa. In certain embodiments the one or more polymer moiety has a maximum molecular weight of 160 kDa. In certain embodiments the one or more polymer moiety has a maximum molecular weight of 120 kDa. In certain embodiments the one or more polymer moiety has a maximum molecular weight of 100 kDa. In certain embodiments the one or more polymer moiety has a maximum molecular weight of 80 kDa. In certain embodiments the one or more polymer moiety has a maximum molecular weight of 70 kDa. In certain embodiments the one or more polymer moiety has a maximum molecular weight of 60 kDa. In certain embodiments the one or more polymer moiety has a maximum molecular weight of 50 kDa. In certain embodiments the one or more polymer moiety has a maximum molecular weight of 40 kDa. In certain embodiments the one or more polymer moiety has a molecular weight of about 450 Da. In certain embodiments the one or more polymer moiety has a molecular weight of about 1 kDa. In certain embodiments the one or more polymer moiety has a molecular weight of about 1.5 kDa. In certain embodiments the one or more polymer moiety has a molecular weight of about 2 kDa. In certain embodiments the one or more polymer moiety has a molecular weight of about 2.5 kDa. In certain embodiments the one or more polymer moiety has a molecular weight of about 3 kDa. In certain embodiments the one or more polymer moiety has a molecular weight of about 3.5 kDa. In certain embodiments the one or more polymer moiety has a molecular weight of about 4 kDa. In certain embodiments the one or more polymer moiety has a molecular weight of about 4.5 kDa. In certain embodiments the one or more polymer moiety has a molecular weight of about 5 kDa. In certain embodiments the one or more polymer moiety has a molecular weight of about 5.5 kDa. In certain embodiments the one or more polymer moiety has a molecular weight of about 6 kDa. In certain embodiments the one or more polymer moiety has a molecular weight of about 6.5 kDa. In certain embodiments the one or more polymer moiety has a molecular weight of about 7 kDa. In certain embodiments the one or more polymer moiety has a molecular weight of about 7.5 kDa. In certain embodiments the one or more polymer moiety has a molecular weight of about 8 kDa. In certain embodiments the one or more polymer moiety has a molecular weight of about 8.5 kDa. In certain embodiments the one or more polymer moiety has a molecular weight of about 9 kDa. In certain embodiments the one or more polymer moiety has a molecular weight of about 9.5 kDa. In certain embodiments the one or more polymer moiety has a molecular weight of about 10 kDa. It is understood that if —R^{d′}—of formula (a-3′) comprises one polymer moiety the minimum and maximum molecular weights provided above apply to this one polymer moiety and if —R^{d′}—of formula (a-3′) comprises more than one polymer moiety the minimum and maximum molecular weights provided above refer to the minimum and maximum molecular weight of all polymer moieties together.

In certain embodiments the one or more polymer moiety of —R^{d′}—of formula (a-3′) has a Flory radius of at least 1.2 nm. In certain embodiments the one or more polymer moiety has a Flory radius of at least 1.5 nm. In certain embodiments the one or more polymer moiety has a Flory radius of at least 2 nm. In certain embodiments the one or more polymer moiety has a Flory radius of at least 2.5 nm. In certain embodiments the one or more polymer moiety has a Flory radius of at least 3 nm. In certain embodiments the one or more polymer moiety has a Flory radius of at least 3.5 nm. In certain embodiments the one or more polymer moiety has a Flory radius of at least 4 nm. In certain embodiments the one or more polymer moiety has a Flory radius of at least 4.5 nm. In certain embodiments the one or more polymer moiety has a Flory radius of at least 5 nm. In certain embodiments the one or more polymer moiety has a Flory radius of no more than 200 nm. In certain embodiments the one or more polymer moiety has a Flory radius of no more than 175 nm. In certain embodiments the one or more polymer moiety has a Flory radius of no more than 150 nm. In certain embodiments the one or more polymer moiety has a Flory radius of no more than 125 nm. In certain embodiments the one or more polymer moiety has a Flory radius of no more than 100 nm. In certain embodiments the one or more polymer moiety has a Flory radius of no more than 75 nm. In certain embodiments the one or more polymer moiety has a Flory radius of no more than 50 nm. In certain embodiments the one or more polymer moiety has a Flory radius of no more than 45 nm. In certain embodiments the one or more polymer moiety has a Flory radius of no more than 40 nm. In certain embodiments the one or more polymer moiety has a Flory radius of no more than 35 nm. In certain embodiments the one or more polymer moiety has a Flory radius of no more than 30 nm. In certain embodiments the one or more polymer moiety has a Flory radius of about 1.2 nm. In certain embodiments the one or more polymer moiety has a Flory radius of about 1.5 nm. In certain embodiments the one or more polymer moiety has a Flory radius of about 2 nm. In certain embodiments the one or more polymer moiety has a Flory radius of about 2.5 nm. In certain embodiments the one or more polymer moiety has a Flory radius of about 3 nm. In certain embodiments the one or more polymer moiety has a Flory radius of about 3.5 nm. In certain embodiments the one or more polymer moiety has a Flory radius of about 4 nm. In certain embodiments the one or more polymer moiety has a Flory radius of about 4.5 nm. In certain embodiments the one or more polymer moiety has a Flory radius of about 5 nm. In certain embodiments the one or more polymer moiety has a Flory radius of about 5.5 nm. In certain embodiments the one or more polymer moiety has a Flory radius of about 6 nm. In certain embodiments the one or more polymer moiety has a Flory radius of about 6.5 nm. In certain embodiments the one or more polymer moiety has a Flory radius of about 7 nm. In certain embodiments the one or more polymer moiety has a Flory radius of about 8.5 nm. In certain embodiments the one or more polymer moiety has a Flory radius of about 9 nm. In certain embodiments the one or more polymer moiety has a Flory radius of about 10 nm. It is understood that if —R^{d′}—of formula (a-3′) comprises one polymer moiety the Flory radius provided above applies to this one polymer moiety and if —R^{d′}—of formula (a-3′) comprises more than one polymer moiety the Flory radius provided above refers to the Flory radius of all polymer moieties together. —R^{d′}—of formula (a-3′) comprises one or more polymer moiety, such as polymer moiety selected from the group consisting of poly(2-methacryloyl-oxyethyl phosphonyl cholines), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycol), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments —R^{d′}—of formula (a-3′) comprises a PEG-based polymer. In certain embodiments —R^{d′}—of formula (a-3′) comprises a hyaluronic acid-based polymer. In certain embodiments —R^{d′}—of formula (a-3′) comprises a random coil polymer. In certain embodiments —R^{d′}—of formula (a-3′) comprises a poly-sarcosine polymer.

In certain embodiments -D- is a protein or peptide drug moiety and -AB² is conjugated to a functional group of -D- provided by the N-terminal amine, the C-terminal carboxyl or a side chain of an amino acid residue. In certain embodiments -AB² is conjugated to the N-terminal amine functional group of -D-. In certain embodiments -AB² is conjugated to the C-terminal carboxyl functional group. In certain embodiments -AB² is conjugated to a functional group provided by an amino acid residue of -D-, such as by a lysine, serine, aspartic acid, glutamic acid, arginine, histidine, threonine, glutamine, asparagine, cysteine, proline, tyrosine or tryptophan. In certain embodiments -AB² is conjugated to the functional group of the side chain of a lysine of -D-. In certain embodiments -AB² is conjugated to the functional group of the side chain of a serine of -D-. In certain embodiments -AB² is conjugated to the functional group of the side chain of an aspartic acid of -D-. In certain embodiments -AB² is conjugated to the functional group of the side chain of a glutamic acid of -D-. In certain embodiments -AB² is conjugated to the functional group of the side chain of an arginine of -D-. In certain embodiments -AB² is conjugated to the functional group of the side chain of a histidine of -D-. In certain embodiments -AB² is conjugated to the functional group of the side chain of a threonine of -D-. In certain embodiments -AB² is conjugated to the functional group of the side chain of a glutamine of -D-. In certain embodiments -AB² is conjugated to the functional group of the side chain of an asparagine of -D-. In certain embodiments -AB² is conjugated to the functional group of the side chain of a cysteine of -D-. In certain embodiments -AB² is conjugated to the functional group of the side chain of a proline of -D-. In certain embodiments -AB² is conjugated to the functional group of the side chain of a tyrosine of -D-. In certain embodiments -AB² is conjugated to the functional group of the side chain of a tryptophan of -D-.

In certain embodiments -D-AB² is selected from the group consisting of semaglutide, liraglutide, ecnoglutide, GZR18, GL0034, tirzepatide, cotadutide, BI-456906, pemvidutide, mazdutide, dapiglutide and retatrutide.

In certain embodiments -D-AB² is selected from the group consisting of semaglutide, liraglutide, ecnoglutide, GZR18 and GL0034.

In certain embodiments -D-AB² is semaglutide. Semaglutide is a compound of formula

(SEQ ID NO: 40)
HX₁EGTFTSDVSSYLEGQAAKEFIAWLVRGRG,

wherein
X₁ is α-aminoisobutyric acid (Aib); and
the lysine at position 20 is chemically modified through conjugation to the epsilon-amine group of the lysine side-chain with

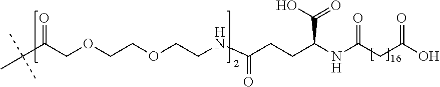

wherein
the dashed line indicates attachment to the epsilon-amine group of the lysine side chain of the lysine at position 20.

Semaglutide may be prepared using methods known to those skilled in the art, such as those described in WO2006/097537.

In certain embodiments -D-AB² is liraglutide. Liraglutide is a compound of formula

(SEQ ID NO: 41)
HAEGTFTSDVSSYLEGQAAKEFIAWLVRGRG,

wherein
the lysine at position 20 is chemically modified through conjugation to the epsilon-amine
group of the lysine side chain with

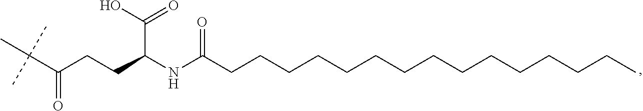

wherein
the dashed line indicates attachment to the epsilon-amine group of the lysine side chain of the lysine at position 20.

In certain embodiments -D-AB² is ecnoglutide. Ecnoglutide is a compound of formula

(SEQ ID NO: 42)
HVEGTFTSDVSSYLEEQAAREFIKWLVRGRG,

wherein
the lysine at position 24 is chemically modified through conjugation to the epsilon-amine group of the lysine side chain with

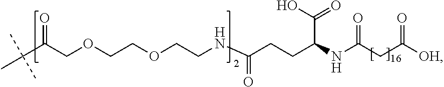

wherein the dashed line indicates attachment to the epsilon-amine group of the lysine side chain of the lysine at position 24.

In certain embodiments -D-AB² is GZR18. GZR18 is a compound of formula

(SEQ ID NO: 43)
HGEGTFTSDVSSYLEGQAAKEFIAWLVRGRG,

wherein
the lysine at position 20 is chemically modified through conjugation to the epsilon-amine group of the lysine side-chain with

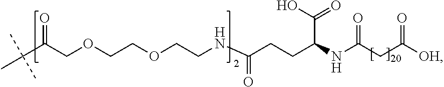

wherein the dashed line indicates attachment to the epsilon amine group of the lysine side chain of the lysine at position 20.

In certain embodiments -D-AB² is GL0034. GL0034 is also known as utreglutide and is a compound of formula

(SEQ ID NO: 44)
HX₁EGTFTSDVSSYLEGQAAKEFIAWLVRGRGL,

wherein
X₁ is Aib; and
the lysine at position 20 is chemically modified through conjugation to the epsilon-amine group of the lysine side-chain with

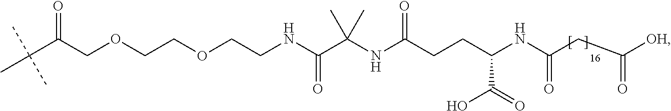

wherein the dashed line indicates attachment to the epsilon amine group of the lysine side chain of the lysine at position 20.

In certain embodiments -D-AB² is a dual GLP-1 receptor agonist selected from the group consisting of tirzepatide, cotadutide, BI-456906, pemvidutide and mazdutide.

In certain embodiments -D-AB² is a dual GLP-1 receptor agonist that activates the GLP-1 receptor and the GIP receptor. An example for such dual GLP-1 receptor agonist is tirzepatide. In certain embodiments -D-AB² is tirzepatide. Tirzepatide is a compound of formula

(SEQ ID NO: 45)
YX₁EGTFTSDYSIX₂LDKIAQKAFVQWLIAGGPSSGAPPPS

wherein
X₁ is Aib;
X₂ is Aib;
the lysine at position 20 is chemically modified through conjugation to the epsilon-amine group of the lysine side-chain with ([2-(2-amino-ethoxy)-ethoxy]-acetyl)₂-(γGlu)₁-CO—(CH₂)₁₈—CO₂H; and
the C-terminal serine, i.e. the serine at position 39, is amidated as a C-terminal primary amide.

The moiety ([2-(2-amino-ethoxy)-ethoxy]-acetyl)₂-(γGlu)₁-CO—(CH₂)₁₈—CO₂H has the following structure

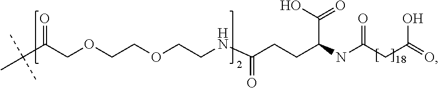

wherein the dashed line indicates attachment to -D-.

In certain embodiments -D-AB² is a dual GLP-1 receptor agonist that activates the GLP-1 receptor and the glucagon receptor selected from the group consisting of cotadutide, BI-456906, pemvidutide and mazdutide.

In certain embodiments -D-AB² is cotadutide. Cotadutide is a compound of formula

(SEQ ID NO: 46)
HSQGTFTSDKSEYLDSERARDFVAWLEAGG,

wherein
the lysine at position 10 is chemically modified through conjugation to the epsilon-amine group of the lysine side-chain with γ-Glu-palmitoyl.

The moiety γ-Glu-palmitoyl has the following structure:

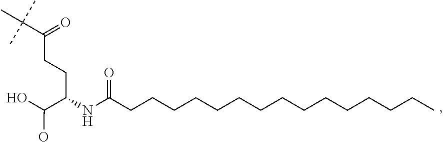

wherein the dashed line indicates attachment to -D-.

In certain embodiments -D-AB² is BI-456906. BI-456906 is also known as survodutide and is a compound of formula

(SEQ ID NO: 47)
HX₁QGTFTSDYSKYLDERAAKDFIKWLESA,

wherein
X₁ is 1-amino-cyclobutanecarboxylic acid (Ac4c);
the lysine at position 24 is chemically modified through conjugation to the epsilon-amine group of the lysine side-chain with [17-carboxy-heptadecanoyl]-isoGlu-GSGSGG; and the C-terminal alanine, i.e. the alanine at position 29, is amidated as a C-terminal primary amide.

The moiety [17-carboxy-heptadecanoyl]-isoGlu-GSGSGG has the following structure:

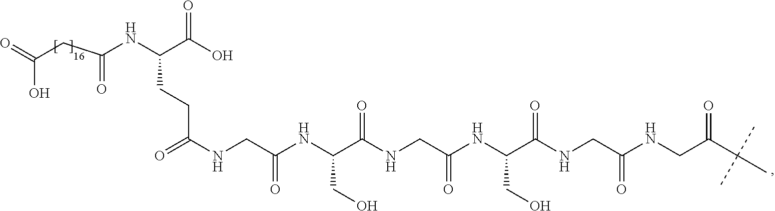

wherein the dashed line indicates attachment to -D-.

In certain embodiments -D-AB² is pemvidutide. Pemvidutide is a compound of formula

(SEQ ID NO: 48)
HX₁QGTFTSDYSKYLDEKAAKEFIQWLLQT,

wherein
X₁ is Aib;
the glutamic acid at position 16 and the lysine at position 20 are connected via a lactam bridge;
the lysine at position 17 is chemically modified through conjugation to the epsilon-amine group of the lysine side-chain with glucuronic acid C-18, which is a moiety of formula

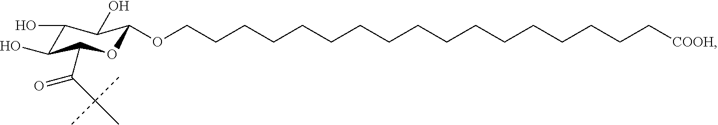

wherein
the dashed line indicates attachment to the epsilon-amine group of the lysine at position 17; and
the C-terminal threonine, i.e., the threonine at position 29, is amidated as a C-terminal primary amide

In certain embodiments -D-AB² is mazdutide. Mazdutide is a compound of formula

(SEQ ID NO: 49)
HX₁QGTFTSDYSKYLDEKKAKEFVEWLLEGGPSSG,

wherein
X₁ is Aib,
the lysine at position 20 is chemically modified by conjugation of the epsilon-amine group of the lysine side chain with ([2-(2-amino-ethoxy)-ethoxy]-acetyl)₂-(γ-Glu)-CO—(CH₂)₁₈—CO₂H; and
the C-terminal glycine, i.e. the glycine at position 34, is amidated.

The moiety ([2-(2-amino-ethoxy)-ethoxy]-acetyl)₂-(γ-Glu)-CO—(CH₂)₁₈—CO₂H has the following structure:

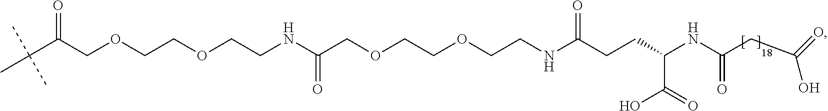

wherein the dashed line indicates attachment to -D-.

In certain embodiments -D-AB² is a dual GLP-1 receptor agonist that activates the GLP-1 receptor and the GLP-2 receptor, such as dapiglutide. Dapiglutide is a compound of formula

(SEQ ID NO: 50)
HX₁EGSFTSELATILDKQAARDFIAWLIQHKITD,

wherein
X₁ is Aib; and
the lysine in position 16 is chemically modified by conjugation of the epsilon-amino-group of the lysine side chain with [17-carboxy-heptadecanoyl]-isoGlu.

The moiety [17-carboxy-heptadecanoyl]-isoGlu has the following structure:

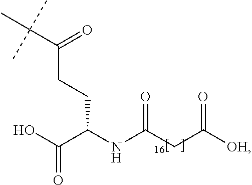

wherein the dashed line indicates attachment to -D-.

In certain embodiments -D-AB² is retatrutide, which is a triple GLP-1 receptor agonist that activates the GLP-1 receptor, the GIP receptor and the GCG receptor. Retatrutide is a compound of formula

(SEQ ID NO: 51)
YX₁QGTFTSDYSIX₂LDKKAQX₃AFIEYLLEGGPSSGAPPPS,

wherein
X₁ is Aib;
X₂ is α-methyl-leucine (αMeL);
X₃ is Aib;
the lysine at position 17 is chemically modified by conjugation of the epsilon-amine group of the lysine side chain with (2-[2-(2-amino-ethoxy)-ethoxy]-acetyl)-(γGlu)-CO—(CH₂)₁₈—CO₂H; and
the C-terminal serine, i.e. the serine at position 39, is amidated.

Retatrutide can also be described as Y-Aib-QGTFTSDYSI-αMeL-LDKK ((2-[2-(2-amino-ethoxy)-ethoxy]-acetyl)-(γGlu)-CO—(CH₂)₁₈—CO₂H) AQ-Aib-AFIEYLLEGGPSSGAPPPS-NH₂ (SEQ ID NO:51).

The moiety (2-[2-(2-amino-ethoxy)-ethoxy]-acetyl)-(γGlu)-CO—(CH₂)₁₈—CO₂H has the following structure:

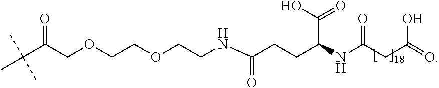

In certain embodiments -D-AB² is noiiglutide, also known as SHR20004.

In certain embodiments -D-AB² is ZT002.

In certain embodiments -D-AB² is of formula (b-1)

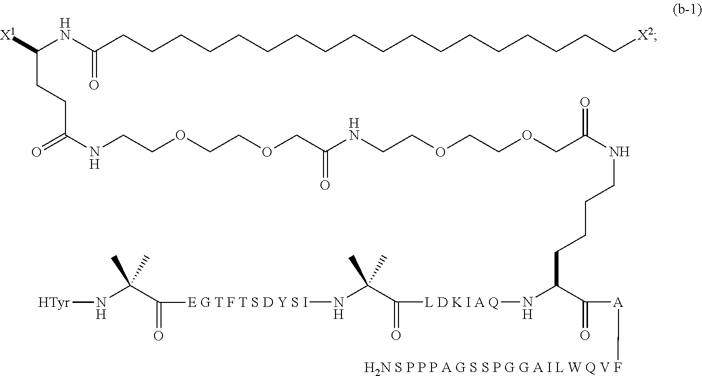

wherein
X¹—is

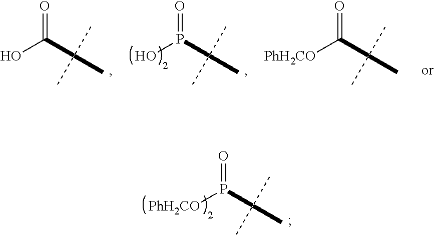

and
—X² is

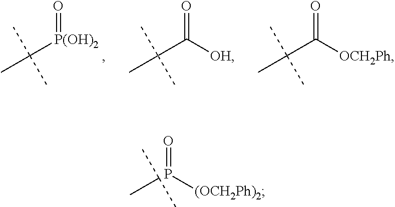

with Ph being phenyl.

The moiety -D- of formula (b-1) has the sequence of SEQ ID NO:57

YX₁EGTFTSDYSIX₂LDKIAQKAFVQWLIAGGPSSGAPPPS,

with X₁ and X₂ being Aib.

The compounds of formula (b-1) are disclosed in WO2022159395 and WO2023/044290, the content of which is herewith incorporated by reference in its entirety.

In certain embodiments -D-AB is of formula (b-1) and X¹—is

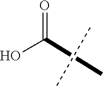

### and —X² is

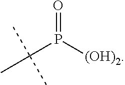

In certain embodiments D-AB is of formula (b-1) and X¹—is

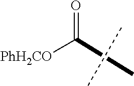

### and —X² is

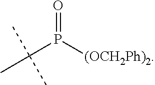

In certain embodiments D-AB is of formula (b-1) and X¹—is

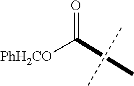

### and —X² is

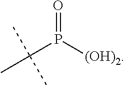

In certain embodiments D-AB is of formula (b-1) and X¹—is

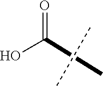

### and —X² is

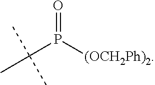

In certain embodiments D-AB is of formula (b-1) and X¹—is

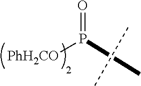

### and —X² is

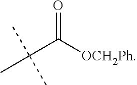

In certain embodiments D-AB is of formula (b-1) and X¹—is

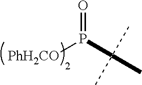

### and —X² is

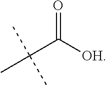

In certain embodiments D-AB is of formula (b-1) and X¹—is

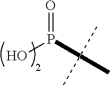

### and —X² is

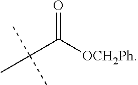

In certain embodiments D-AB is of formula (b-1)

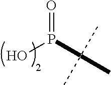

### and X¹—is

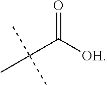

In certain embodiments D-AB is of formula (b-1) and X¹ is

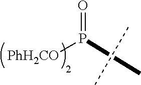

### and X¹—is

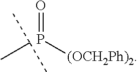

In certain embodiments D-AB is of formula (b-1) and X¹—is

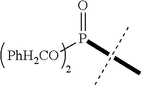

### and —X² is

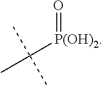

In certain embodiments D-AB is of formula (b-1) and X¹—is

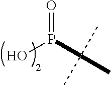

### and —X₂ is

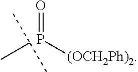

In certain embodiments D-AB is of formula (b-1) and X¹—is

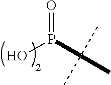

### and —X₂ is

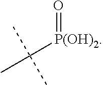

In certain embodiments -D-AB² is selected from the group consisting of semaglutide, liraglutide, ecnoglutide, GZR18, GL0034, tirzepatide, cotadutide, BI-456906, pemvidutide, mazdutide, dapiglutide and retatrutide.

In certain embodiments -D-AB² is of formula

(SEQ ID NO: 52)
k(γE-(miniPEG)₂-γE-COC₁₆H₃₂CO₂H)(N-Me)GSVSEIQLMHNL
GKHLNSMERVEWLRKKLQDVHK(γE-(miniPEG)₂-γE-
COC₁₆H₃₂CO₂H)-OH,

wherein
k is d-Lys;
γE is the 1-isomer of gamma, glutamic acid;
miniPEG is COCH₂OCH₂CH₂OCH₂CH₂NH;
COC₁₆H₃₂CO₂H is C18 diacid;
(N-Me)G is sarcosine;
K is 1-isomer of lysine; and
—OH designates the C-terminal amino acid has a terminal carboxylic acid.

In certain embodiments -D-AB² is of formula

(SEQ ID NO: 53)
k(γE-(miniPEG)₂-γE-COC₁₆H₃₂CO₂H)(N-Me)GSVSEIQLMHNL
GKHLNSMERVEWLRKKLQDVHK(γE-(miniPEG)₂-γE-
COC₁₆H₃₂CO₂H)-OH,

wherein
k is d-Lys;
γE is the 1-isomer of gamma, glutamic acid;
(miniPEG)₂ is COCH₂OCH₂CH₂OCH₂CH₂NH;
COC₁₆H₃₂CO₂H is C₁₈ diacid;
(N-Me)G is sarcosine;
K is 1-isomer of lysine; and
—OH designates the C-terminal amino acid has a terminal carboxylic acid.

If -D- is a peptide or protein drug moiety, -L¹- is either conjugated to a functional group of a side chain of an amino acid residue of -D-, to the N-terminal amine functional group or to the C-terminal carboxyl functional group of -D- or to a nitrogen atom in the backbone chain of -D-.

If -D- is a peptide or protein drug moiety, -L¹- is in certain embodiments conjugated to a functional group of -D- selected from the group consisting of carboxylic acid, primary amine, secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, sulfate, disulfide, vinyl sulfone, vinyl ketone, diazoalkane, oxirane, guanidine and aziridine. If -D- is a peptide or protein drug moiety, -L¹- is in certain embodiments conjugated to a functional group of -D- selected from the group consisting of hydroxyl, primary amine, secondary amine and guanidine. If -D- is a peptide or protein drug moiety, -L¹- is in certain embodiments conjugated to a functional group of -D- selected from the group consisting of primary amine and secondary amine. If -D- is a peptide or protein drug moiety, -L¹- is in certain embodiments conjugated to a primary amine of -D-.

If -D- is a peptide or protein drug moiety, -L¹- may be conjugated to a functional group of the side chain of an amino acid residue of -D-, which may be a proteinogenic amino acid residue or a non-proteinogenic amino acid residue.

If -D- is a peptide or protein drug moiety, -L¹- is in certain embodiments conjugated to a functional group of the side chain of a proteinogenic amino acid residue of -D-. In certain embodiments such amino acid residue is selected from the group consisting of histidine, lysine, tryptophan, serine, threonine, tyrosine, aspartic acid, glutamic acid and arginine. In certain embodiments such amino acid residue is selected from the group consisting of lysine, aspartic acid, arginine and serine. In certain embodiments such amino acid residue is selected from the group consisting of lysine, arginine and serine.

If -D- is a peptide or protein drug moiety, -L¹- is in certain embodiments conjugated to a functional group of a lysine residue of -D-. If -D- is a peptide or protein drug moiety, -L¹- is in certain embodiments conjugated to a functional group of a histidine residue of -D-. If -D- is a peptide or protein drug moiety, -L¹- is in certain embodiments conjugated to a functional group of a tryptophan residue of -D-. If -D- is a peptide or protein drug moiety, -L¹- is in certain embodiments conjugated to a functional group of a serine residue of -D-. If -D- is a peptide or protein drug moiety, -L¹- is in certain embodiments conjugated to a functional group of a threonine residue of -D-. If -D- is a peptide or protein drug moiety, -L¹- is in certain embodiments conjugated to a functional group of a tyrosine residue of -D-. If -D- is a peptide or protein drug moiety, -L¹- is in certain embodiments conjugated to a functional group of an aspartic acid residue of -D-. If -D- is a peptide or protein drug moiety, -L¹- is in certain embodiments conjugated to a functional group of a glutamic acid residue of -D-. If -D- is a peptide or protein drug moiety, -L¹- is in certain embodiments conjugated to a functional group of an arginine residue of -D-.

If -D- is a peptide or protein drug moiety, -L¹- is in certain embodiments conjugated to a functional group of the side chain of a non-proteinogenic amino acid residue of -D-.

It is understood that not every peptide or protein drug moiety -D- may comprise all of these amino acid residues.

If -D- is a peptide or protein drug moiety, -L¹- is in certain embodiments conjugated to the N-terminal amine functional group of -D-.

If -D- is a peptide or protein drug moiety, -L¹- is in certain embodiments conjugated to the C-terminal functional group of -D-.

The moiety -L¹- may be connected to -D- through any type of linkage, provided that it is reversible. In certain embodiments -L¹- is connected to -D- through a linkage selected from the group consisting of amide, ester, carbamate, acetal, aminal, imine, oxime, hydrazone, disulfide and acylguanidine. In certain embodiments -L¹- is connected to -D- through a linkage selected from the group consisting of amide, ester, carbamate and acylguanidin. It is understood that some of these linkages per se are not reversible, but that in the present invention neighboring groups present in -L¹- render these linkages reversible.

In certain embodiments -L¹- is connected to -D- through an amide linkage. In certain embodiments -L¹- is connected to -D- through a carbamate linkage. In certain embodiments -L¹- is connected to -D- through an ester linkage. In certain embodiments -L¹- is connected to -D- through an acylguanidine linkage.

The moiety -L¹- is a reversible prodrug linker from which the drug, i.e., H-D-AB², is released in its free form, i.e. it is a traceless prodrug linker. It is understood that the “H—” in “H-D-AB” is a hydrogen. Suitable prodrug linkers are known in the art, such as for example the reversible prodrug linker moieties disclosed in WO 2005/099768 A2, WO 2006/136586 A2, WO 2011/089216 A1 and WO 2013/024053 A1, which are incorporated by reference herewith.

In certain embodiments -L¹- is disclosed in WO 2009/095479 A2. Accordingly, in certain embodiments the moiety -L¹- is of formula (II):

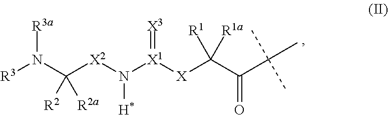

wherein the dashed line indicates attachment to a nitrogen, hydroxyl or thiol of -D-;
—X— is selected from the group consisting of —C(R⁴R^{4a})—; —N(R⁴)—; —O—; —C(R⁴R^{4a})—; C(R⁵R^{5a})—; —C(R⁵R^{5a})—C(R⁴R^{4a})—; —C(R⁴R^{4a})—N(R⁶)—; —N(R⁶)—C(R⁴R^{4a})—; C(R⁴R^{4a})—O—; —O—C(R⁴R^{4a})—; and —C(R⁷R^{7a})—; X₁ is selected from the group consisting of C; and S(O); —X₂— is selected from the group consisting of —C(R⁸R^{8a})—; and —C(R⁸R^{8a})—C(R⁹R^{9a})—; ═X₃ is selected from the group consisting of ═O; ═S; and ═N—CN;
—R¹, —R^{1a}, —R², —R^{2a}, —R⁴, —R^{4a}, —R^{5a}, —R⁶, —R⁸, —R^{8a}, —R⁹, and —R^{9a} are independently selected from the group consisting of —H; and C₁₋₆ alkyl;
—R³, and —R^{3a} are independently selected from the group consisting of —H; and C₁₋₆ alkyl, provided that in case one of —R³, —R^{3a} or both are other than —H they are connected to N to which they are attached through an SP³-hybridized carbon atom;
—R⁷ is selected from the group consisting of —N(R¹⁰R^{10a}); and —NR¹⁰—(C═O)—R¹¹; —R^{7a}, —R¹⁰, —R^{10a}, and —R¹¹ are independently of each other selected from the group consisting of —H; and C₁₋₆ alkyl;
optionally, one or more of the pairs —R^{1a}/—R^{4a}, —R^{1a}/—R^{5a}, —R^{1a}/—R^{7a}, —R^{4a}/—R^{5a}, and —R^{8a}/—R^{9a} form a chemical bond;
optionally, one or more of the pairs -R/—R^{1a}, —R²/—R^{2a}, —R⁴/—R^{4a}, —R⁵/—R^{5a}, —R⁸/—R^{8a}, and —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹, and —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with at least one -L²- and wherein -L¹- is optionally further substituted, provided that the hydrogen marked with the asterisk in formula (II) is not replaced by -L²- or a substituent.

In certain embodiments -L¹- of formula (II) is substituted with one moiety -L²-. In certain embodiments -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles may be formed in which the atoms directly attached to the nitrogen are SP³-hybridized carbon atoms. In other words, such 3- to 10-membered heterocycle formed by —R³/—R^{3a} together with the nitrogen atom to which they are attached has the following structure:

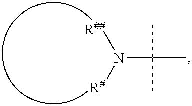

wherein
the dashed line indicates attachment to the rest of -L¹-;
the ring comprises 3 to 10 atoms comprising at least one nitrogen; and
R^{#} and R^{##} represent an sp³-hybridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

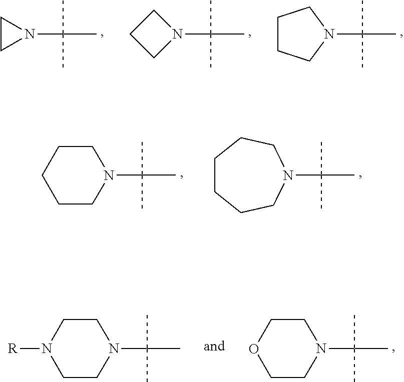

wherein
dashed lines indicate attachment to the rest of the molecule; and
—R is selected from the group consisting of —H and C₁₋₆ alkyl.
L¹- of formula (II) may optionally be further substituted. In general, any substituent may be used as far as the cleavage principle is not affected, i.e., the hydrogen marked with the asterisk in formula (II) is not replaced and the nitrogen of the moiety

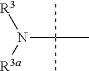

of formula (II) remains part of a primary, secondary or tertiary amine, i.e., —R³ and —R^{3a} are independently of each other —H or are connected to —N< through an sp³-hybridized carbon atom.

In one embodiment —R¹ or —R^{1a} of formula (II) is substituted with -L²-. In another embodiment —R² or —R^{2a} of formula (II) is substituted with -L²-. In another embodiment —R³ or —R^{3a} of formula (II) is substituted with -L²-. In another embodiment —R⁴ of formula (II) is substituted with -L²-. In another embodiment —R⁵ or —R^{5a} of formula (II) is substituted with -L²-. In another embodiment —R⁶ of formula (II) is substituted with -L²-. In another embodiment —R⁷ or —R^{7a} of formula (II) is substituted with -L²-. In another embodiment —R⁸ or —R^{8a} of formula (II) is substituted with -L²-. In another embodiment —R⁹ or —R^{9a} of formula (II) is substituted with -L²-. In another embodiment —R¹⁰ is substituted with -L²-. In another embodiment —R¹¹ is substituted with -L²-. In certain embodiments —R³ of formula (II) is substituted with -L²-.

In certain embodiments —X—of formula (II) is selected from the group consisting of —C(R⁴R^{4a})—, —N(R⁴)—and —C(R⁷R^{7a})—. In certain embodiments —X—of formula (II) is —C(R⁴R^{4a})—. In certain embodiments —X—of formula (II) is —C(R⁷R^{7a})—.

In certain embodiments —R⁷ of formula (TT) is —NR¹⁰—(C═O)—R¹¹.

In certain embodiments —R^{7a} of formula (II) is selected from —H, methyl and ethyl. In certain embodiments —R^{7a} of formula (II) is —H.

In certain embodiments —R¹⁰ is selected from —H, methyl and ethyl. In certain embodiments —R¹⁰ is methyl.

In certain embodiments —R¹¹ is selected from —H, methyl and ethyl. In certain embodiments —R¹¹ is —H. In certain embodiments —R¹¹ is substituted with -L²-.

In certain embodiments —X—of formula (II) is —N(R⁴)—.

In certain embodiments —R⁴ is selected from the group consisting of —H, methyl and ethyl. In certain embodiments —R⁴ is —H.

In certain embodiments X¹ of formula (II) is C.

In certain embodiments ═X₃ of formula (II) is ═O.

In certain embodiments —X²—of formula (II) is —C(R⁸R^{8a})—.

In certain embodiments —R⁸ and —R^{8a} of formula (II) are independently selected from the group consisting of —H, methyl and ethyl. In certain embodiments at least one of —R⁸ and —R^{8a} of formula (II) is —H. In certain embodiments both —R⁸ and —R^{8a} of formula (II) are —H.

In certain embodiments —R¹ and —R^{1a} of formula (II) are independently selected from the group consisting of —H, methyl and ethyl.

In certain embodiments at least one of —R¹ and —R^{1a} of formula (II) is —H. In certain embodiments —R¹ and —R^{1a} of formula (II) are —H.

In certain embodiments at least one of —R¹ and —R^{1a} of formula (II) is methyl. In certain embodiments both —R¹ and —R^{1a} of formula (TT) are methyl.

In certain embodiments —R² and —R^{2a} of formula (II) are independently selected from the group consisting of —H, methyl and ethyl. In certain embodiments at least one of —R² and —R^{2a} of formula (II) is —H. In certain embodiments both —R² and —R^{2a} of formula (II) are H.

In certain embodiments —R³ and —R^{3a} of formula (II) are independently selected from the group consisting of —H, methyl, ethyl, propyl and butyl.

In certain embodiments at least one of —R³ and —R^{3a} of formula (II) is methyl. In certain embodiments —R³ of formula (II) is methyl and —R^{3a} of formula (II) is —H.

In certain embodiments —R³ and —R^{3a} of formula (II) are both —H.

In certain embodiments -D- is connected to -L¹- through a nitrogen by forming an amide bond.

In certain embodiments -L¹- is of formula (II), wherein X is —C(R⁷R^{7a})—; X¹ is C; —X²— is —C(R⁸R^{8a})—C(R⁹R^{9a})—; ═X³ is ═O; —R¹ and —R^{1a} are —H; —R² and —R^{2a} are —H; —R³ and —R^{3a} are methyl; —R⁷ is —N(R¹⁰R^{10a}); —R^{7a} is —H; —R⁸, —R^{8a}, —R⁹ and —R^{9a} are —H; and —R¹⁰ is methyl and —R^{10a} is —H.

In certain embodiments -L¹- is of formula (IIa)

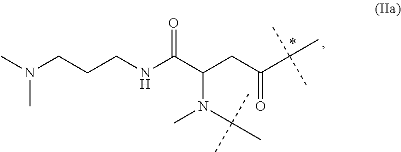

wherein
the dashed line marked with the asterisk indicates attachment to a nitrogen of -D-; and
the unmarked dashed line indicates attachment to -L²-.

In certain embodiments -L¹- is of formula (IIa-a)

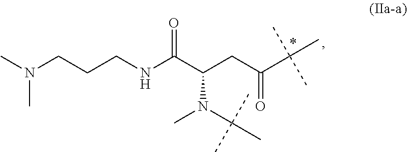

wherein
the dashed line marked with the asterisk indicates attachment to a nitrogen of -D-; and
the unmarked dashed line indicates attachment to -L²-.

In certain embodiments -L¹- is of formula (IIa-b)

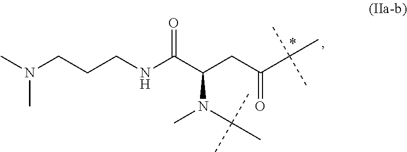

wherein
the dashed line marked with the asterisk indicates attachment to a nitrogen of -D-; and
the unmarked dashed line indicates attachment to -L²-.

The moiety of formula (IIa), (IIa-a) and (IIa-b) is connected to -D- via an amide bond formed by a nitrogen of an amine functional group and the carbonyl to the left of the dashed line marked with the asterisk.

In certain embodiments -L¹- is of formula (II), wherein X is —C(R⁷R^{7a})—; X¹ is C; —X²— is —C(R⁸R^{8a})—C(R⁹R^{9a})—; ═X³ is ═O; —R¹ and —R^{1a} are —H; —R² and —R^{2a} are —H; —R³ and —R^{3a} are methyl; —R⁷ is —NR¹⁰—(C═O)—R¹¹; —R^{7a} is —H; —R⁸, —R^{8a}, —R⁹ and —R^{9a} are —H; and —R¹⁰ is methyl and —R¹¹ is —H.

In certain embodiments -L¹- is of formula (II), wherein X is —C(R⁷R^{7a})—; X¹ is C; —X²— is —C(R⁸R^{8a})—C(R⁹R^{9a})—; ═X³ is ═O; —R¹ and —R^{1a} are —H; —R² and —R^{2a} are —H; —R³ and —R^{3a} are methyl; —R⁷ is —NR¹⁰—(C═O)—R¹¹; —R^{7a} is —H; —R⁸, —R^{8a}, —R⁹ and —R^{9a} are —H; and —R¹⁰ is —H and —R¹¹ is —H.

In certain embodiments -L¹- is of formula (IIab)

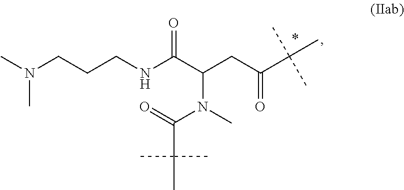

wherein
the dashed line marked with the asterisk indicates attachment to a nitrogen of -D-; and
the unmarked dashed line indicates attachment to -L²-.

In certain embodiments -L¹- is of formula (IIab-a)

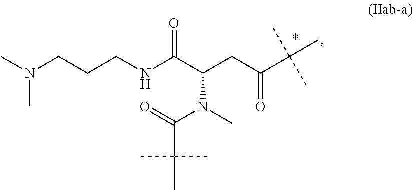

wherein
the dashed line marked with the asterisk indicates attachment to a nitrogen of -D-; and
the unmarked dashed line indicates attachment to -L²-.

In certain embodiments -L¹- is of formula (IIab-b)

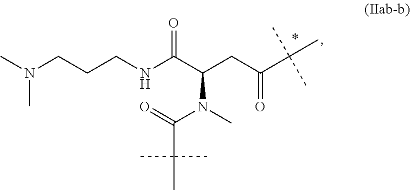

wherein
the dashed line marked with the asterisk indicates attachment to a nitrogen of -D-; and
the unmarked dashed line indicates attachment to -L²-.

The moiety of formula (IIab), (IIab-a) and (IIab-b) is connected to -D- via an amide bond formed by a nitrogen of an amine functional group and the carbonyl to the left of the dashed line marked with the asterisk.

In certain embodiments -L¹- is disclosed in WO2016/020373A1. Accordingly, in certain embodiments the moiety -L¹- is of formula (III):

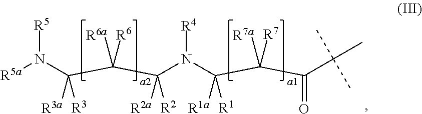

wherein
the dashed line indicates attachment to a primary or secondary amine or hydroxyl of -D- through an amide or ester linkage, respectively;
—R¹, —R^{1a}, —R², —R^{2a}—R³ and —R^{3a} are independently of each other selected from the group consisting of —H, —C(R⁸R^{8a}R^{8b}), —C(═O)R⁸, —C≡N, —C(═NR⁸)R^{8a}, —CR⁸(═CR^{8a}R^{8b}), —C≡CR⁸ and -T;
—R⁴, —R⁵ and —R^{5a}; are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, and —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O) N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—; each —R¹⁰, —R^{10a}, and —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, and —R^{13b} is independently selected from the group consisting of —H, and C₁₋₆ alkyl; wherein C₁₋₆ alkyl is optionally substituted with one or more halogen, which are the same or different;
optionally, one or more of the pairs -R/—R^{1a}, —R²/—R^{2a}, —R³/—R^{3a}, —R⁶/—R^{6a}, and —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, and —R⁶/—R⁷ are joint together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with at least one -L²- and wherein -L¹- is optionally further substituted.

The optional further substituents of -L¹- of formula (III) are in certain embodiments as described above.

In certain embodiments -L¹- of formula (III) is substituted with one moiety -L²-.

In certain embodiments -L¹- of formula (III) is not further substituted.

In certain embodiments -L¹- is as disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

In certain embodiments -L¹- is as disclosed in U.S. Pat. No. 8,946,405B2 and U.S. Pat. No. 8,754,190B2, which are herewith incorporated by reference in their entirety. Accordingly, in certain embodiments -L¹- is of formula (IV):

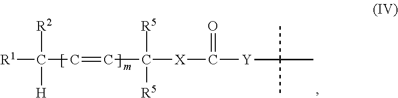

wherein
the dashed line indicates attachment to -D- and wherein attachment is through a functional group of -D- selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴, one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂—and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring;
wherein -L¹- is substituted with at least one -L²- and wherein -L¹- is optionally further substituted.

Only in the context of formula (IV) the terms used have the following meaning:

The term “alkyl” as used herein includes linear, branched or cyclic saturated hydrocarbon groups of 1 to 8 carbons, or in certain embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, such as 6 to 10 carbons, including groups such as phenyl, naphthyl, and anthracenyl. The term “heteroaryl” includes aromatic rings comprising 3 to 15 carbons containing at least one N, O or S atom, such as 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂N R², wherein each R is independently alkyl, alkenyl, alkynyl, aryl or heteroaryl, or two R groups taken together with the atoms to which they are attached form a ring.

In certain embodiments -L¹- of formula (IV) is substituted with one moiety -L²-.

In certain embodiments -L¹- of formula (IV) is not further substituted.

In certain embodiments -L¹- is as disclosed in WO2013/036857A1, which is herewith incorporated by reference in its entirety. Accordingly, in certain embodiments -L¹- is of formula (V):

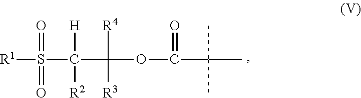

wherein
the dashed line indicates attachment to -D- through an amine functional group of H-D-AB²;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with at least one -L²- and wherein -L¹- is optionally further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, such as 6-10 carbons, including groups such as phenyl, naphthyl, and anthracene “Heteroaryl” includes aromatic rings comprising 3-15 carbons containing at least one N, O or S atom, such as 3-7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments -L¹- of formula (V) is substituted with one moiety -L²-.

In certain embodiments -L¹- of formula (V) is not further substituted.

In certain embodiments -L¹- is as disclosed in U.S. Pat. No. 7,585,837B2, which is herewith incorporated by reference in its entirety. Accordingly, in certain embodiments -L¹- is of formula (VI):

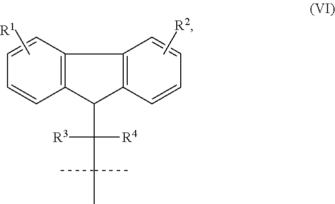

wherein
the dashed line indicates attachment to -D- through an amine functional group of
H-D-AB²;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with at least one -L²- and wherein -L¹- is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, such as 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (VI) is substituted with one moiety -L²-.

In certain embodiments -L¹- of formula (VI) is not further substituted.

A further preferred embodiment for -L¹- is disclosed in WO2002/089789A1, which is herewith incorporated by reference in its entirety. Accordingly, a preferred moiety -L¹- is of formula (VII):

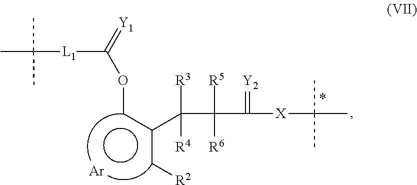

wherein
the dashed line indicates attachment to -D- through an amine functional group of
H-D-AB²;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with at least one -L²- and wherein -L¹- is optionally further substituted.

Only in the context of formula (VII) the terms used have the following meaning:

The term “alkyl” shall be understood to include, e.g., straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophene; alkoxy includes moieties such as methoxy; and phenoxy includes moieties such as 3-nitrophenoxy. Halo—shall be understood to include fluoro, chloro, iodo and bromo.

In certain embodiments -L¹- of formula (VII) is substituted with one moiety -L².

In certain embodiments -L¹- of formula (VII) is not further substituted.

In certain embodiments -L¹- comprises a substructure of formula (VIII)

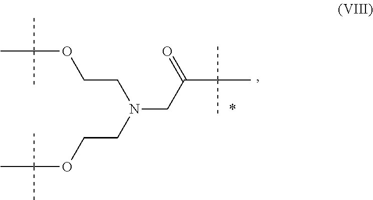

wherein
the dashed line marked with the asterisk indicates attachment to a nitrogen of -D- through an amide bond;
the unmarked dashed lines indicate attachment to the remainder of -L¹-; and
wherein -L¹- is substituted with at least one -L²- and wherein -L¹- is optionally further substituted.

In certain embodiments -L¹- of formula (VIII) is substituted with one moiety -L²-.

In certain embodiments -L¹- of formula (VIII) is not further substituted.

In certain embodiments -L¹- comprises a substructure of formula (IX)

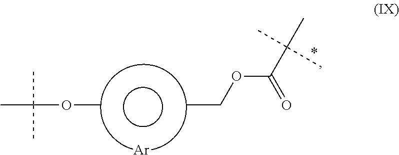

wherein
the dashed line marked with the asterisk indicates attachment to a nitrogen of -D- through a carbamate bond;
the unmarked dashed lines indicate attachment to the remainder of -L¹-; and
wherein -L¹- is substituted with at least one -L²- and wherein -L¹- is optionally further substituted.

In certain embodiments -L¹- of formula (IX) is substituted with one moiety -L²-. In certain embodiments -L¹- of formula (IX) is not further substituted.

In certain embodiments -L¹- has a structure as disclosed in WO2020/206358 A1. Accordingly, in certain embodiments the moiety -L¹- is of formula (X):

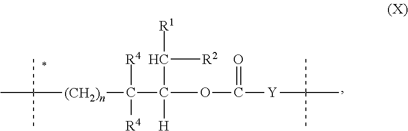

wherein
the unmarked dashed line indicates attachment to -D-;
the dashed line marked with the asterisk indicates attachment to -L²-;
n is an integer selected from the group consisting of 0, 1, 2, 3, 4, 5 and 6;
—R¹ and —R² are independently an electron-withdrawing group, alkyl, or —H, and wherein at least one of —R¹ or —R² is an electron-withdrawing group;
each —R⁴ is independently C₁-C₃ alkyl or the two —R⁴ are taken together with the carbon atom to which they are attached to form a 3- to 6-membered ring; and
—Y— is absent when -D- is a drug moiety connected through an amine, or —Y— is —N(R⁶)CH₂—when -D- is a drug moiety connected through a phenol, alcohol, thiol, thiophenol, imidazole, or non-basic amine; wherein —R⁶ is optionally substituted C₁-C₆ alkyl, optionally substituted aryl, or optionally substituted heteroaryl.

In certain embodiments n of formula (X) is an integer selected from 1, 2, 3, 4, 5 and 6. In certain embodiments n of formula (X) is an integer selected from 1, 2 and 3. In certain embodiments n of formula (X) is an integer from 0, 1, 2 and 3. In certain embodiments n of formula (X) is 1. In certain embodiments n of formula (X) is 2. In certain embodiments n of formula (X) is 3.

In certain embodiments the electron-withdrawing group of —R¹ and —R² of formula (X) is selected from the group consisting of —CN; —NO₂; optionally substituted aryl; optionally substituted heteroaryl; optionally substituted alkenyl; optionally substituted alkynyl; —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments the electron-withdrawing group of —R¹ and —R² of formula (X) is —CN. In certain embodiments the electron-withdrawing group of —R¹ and —R² of formula (X) is —NO₂. In certain embodiments the electron-withdrawing group of —R¹ and —R² of formula (X) is optionally substituted aryl comprising 6 to 10 carbons. In certain embodiments the electron-withdrawing group of —R¹ and —R² of formula (X) is optionally substituted phenyl, naphthyl, or anthracenyl. In certain embodiments the electron-withdrawing group of —R¹ and —R² of formula (X) is optionally substituted heteroaryl comprising 3 to 7 carbons and comprising at least one N, O, or S atom. In certain embodiments the electron-withdrawing group of —R¹ and —R² of formula (X) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of —R¹ and —R² of formula (X) is optionally substituted alkenyl containing 2 to 20 carbon atoms. In certain embodiments the electron-withdrawing group of —R¹ and —R² of formula (X) is optionally substituted alkynyl comprising 2 to 20 carbon atoms. In certain embodiments the electron-withdrawing group of —R¹ and —R² of formula (X) is —COR³, —SOR³, or —SO₂R³, wherein —R³ is —H, optionally substituted alkyl comprising 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl comprising 1 to 20 carbon atoms, or both —R⁸ groups are taken together with the nitrogen to which they are attached to form a heterocyclic ring. In certain embodiments the electron-withdrawing group of —R¹ and —R² of formula (X) is —SR⁹, wherein —R⁹ is optionally substituted alkyl comprising 1 to 20 carbon atoms, optionally substituted aryl, optionally substituted arylalkyl, optionally substituted heteroaryl, or optionally substituted heteroarylalkyl.

In certain embodiments at least one of —R¹ or —R² of formula (X) is —CN, —SOR³ or —SO₂R³. In certain embodiments at least one of —R¹ and —R² of formula (X) is —CN or —SO₂R³. In certain embodiments at least one of —R¹ and —R² of formula (X) is —CN or —SO₂R³, wherein —R³ is optionally substituted alkyl, optionally substituted aryl, or —NR⁸₂. In certain embodiments at least one of —R¹ and —R² of formula (X) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments each —R⁴ of formula (X) is independently C₁-C₃ alkyl. In certain embodiments both —R⁴ are methyl.

In certain embodiments —Y—of formula (X) is absent. In certain embodiments —Y—of formula (X) is —N(R⁶)CH₂—.

In certain embodiments -L¹- is of formula (X), wherein n is 1, —R¹ is —CN, —R² is —H, and —R⁴ is —CH₃. In certain embodiments -L¹- is of formula (X), wherein n is 1, —R¹ is —SO₂N(CH₃)₂, —R² is —H, and —R⁴ is —CH₃. In certain embodiments -L¹- is of formula (X), wherein n is 1, —R¹ is SO₂CH₃, —R² is —H, and —R⁴ is —CH₃. In certain embodiments -L¹- is of formula (X), wherein n is 1, —R¹ is —SO₂N(CH₂CH₂)₂CHCH₃, —R² is —H, and —R⁴ is —CH₃. In certain embodiments -L¹- is of formula (X), wherein n is 1, —R¹ is phenyl substituted with —SO₂, —R² is —H, and —R⁴ is —CH₃. In certain embodiments -L¹- is of formula (X), wherein n is 1, —R¹ is phenyl substituted with —SO₂ and —Cl, —R² is —H, and —R⁴ is —CH₃. In certain embodiments -L¹- is of formula (X), wherein n is 1, —R¹ is —SO₂N(CH₂CH₂)₂₀, —R² is —H, and —R⁴ is —CH₃. In certain embodiments -L¹- is of formula (X), wherein n is 1, —R¹ is —SO₂CH(CH₃)₂, —R² is —H, and —R⁴ is —CH₃. In certain embodiments -L¹- is of formula (X), wherein n is 1, —R¹ is —SO₂N(CH₃)(CH₂CH₃), —R² is —H, and —R⁴ is —CH₃. In certain embodiments -L¹- is of formula (X), wherein n is 1, —R¹ is —SO₂N(CH₂CH₂OCH₃)₂, —R² is —H, and —R⁴ is —CH₃. In certain embodiments -L¹- is of formula (X), wherein n is 1, —R¹ is phenyl substituted with —SO₂ and —CH₃, —R² is —H, and —R⁴ is —CH₃.

In certain embodiments -L¹- is of formula (X), wherein n is 2, —R¹ is —CN, —R² is —H, and —R⁴ is —CH₃. In certain embodiments -L¹- is of formula (X), wherein n is 2, —R¹ is —SO₂N(CH₃)₂, —R² is —H, and —R⁴ is —CH₃. In certain embodiments -L¹- is of formula (X), wherein n is 2, —R¹ is SO₂CH₃, —R² is —H, and —R⁴ is —CH₃. In certain embodiments -L¹- is of formula (X), wherein n is 2, —R¹ is —SO₂N(CH₂CH₂)₂CHCH₃, —R² is —H, and —R⁴ is —CH₃. In certain embodiments -L¹- is of formula (X), wherein n is 2, —R¹ is phenyl substituted with —SO₂, —R² is —H, and —R⁴ is —CH₃. In certain embodiments -L¹- is of formula (X), wherein n is 2, —R¹ is phenyl substituted with —SO₂ and —Cl, —R² is —H, and —R⁴ is —CH₃. In certain embodiments -L¹- is of formula (X), wherein n is 2, —R¹ is —SO₂N(CH₂CH₂)₂₀, —R² is —H, and —R⁴ is —CH₃. In certain embodiments -L¹- is of formula (X), wherein n is 2, —R¹ is —SO₂CH(CH₃)₂, —R² is —H, and —R⁴ is —CH₃. In certain embodiments -L¹- is of formula (X), wherein n is 2, —R¹ is —SO₂N(CH₃)(CH₂CH₃), —R² is —H, and —R⁴ is —CH₃. In certain embodiments -L¹- is of formula (X), wherein n is 2, —R¹ is —SO₂N(CH₂CH₂OCH₃)₂, —R² is —H, and —R⁴ is —CH₃. In certain embodiments -L¹- is of formula (X), wherein n is 2, —R¹ is phenyl substituted with —SO₂ and —CH₃, —R² is —H, and —R⁴ is —CH₃.

In certain embodiments -L¹- is of formula (X), wherein n is 3, —R¹ is —CN, —R² is —H, and —R⁴ is —CH₃. In certain embodiments -L¹- is of formula (X), wherein n is 3, —R¹ is —SO₂N(CH₃)₂, —R² is —H, and —R⁴ is —CH₃. In certain embodiments -L¹- is of formula (X), wherein n is 3, —R¹ is SO₂CH₃, —R² is —H, and —R⁴ is —CH₃. In certain embodiments -L¹- is of formula (X), wherein n is 3, —R¹ is —SO₂N(CH₂CH₂)₂CHCH₃, —R² is —H, and —R⁴ is —CH₃. In certain embodiments -L¹- is of formula (X), wherein n is 3, —R¹ is phenyl substituted with —SO₂, —R² is —H, and —R⁴ is —CH₃. In certain embodiments -L¹- is of formula (X), wherein n is 3, —R¹ is phenyl substituted with —SO₂ and —Cl, —R² is —H, and —R⁴ is —CH₃. In certain embodiments -L¹- is of formula (X), wherein n is 3, —R¹ is —SO₂N(CH₂CH₂)₂₀, —R² is —H, and —R⁴ is —CH₃. In certain embodiments -L¹- is of formula (X), wherein n is 3, —R¹ is —SO₂CH(CH₃)₂, —R² is —H, and —R⁴ is —CH₃. In certain embodiments -L¹- is of formula (X), wherein n is 3, —R¹ is —SO₂N(CH₃)(CH₂CH₃), —R² is —H, and —R⁴ is —CH₃. In certain embodiments -L¹- is of formula (X), wherein n is 3, —R¹ is —SO₂N(CH₂CH₂OCH₃)₂, —R² is —H, and —R⁴ is —CH₃. In certain embodiments -L¹- is of formula (X), wherein n is 3, —R¹ is phenyl substituted with —SO₂ and —CH₃, —R² is —H, and —R⁴ is —CH₃.

Only in the context of formula (X) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n-octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In certain embodiments -L¹- has a structure as disclosed in formula I of WO2021/242756 A1. Accordingly, in certain embodiments the moiety -L¹- is of formula (XIIa):

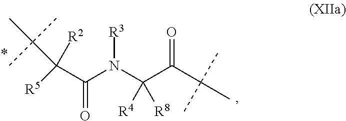

wherein
the unmarked dashed line indicates attachment to -D-;
the dashed line marked with the asterisk indicates attachment to -L²-;
—R², —R⁴ and —R⁸ are independently selected from the group consisting of —H or C₁₋₄ alkyl;
—R³ is C₁₋₄ alkyl or —R³ and —R⁴ together with the atoms to which they are attached form a 5- or 6-membered heterocyclic ring;
—R⁵ is —NH₂;
with the proviso that when —R⁴ and —R³ together with the atoms to which they are attached from a 5- or 6-membered heterocyclic ring —R² is not —H; and
wherein the -L¹- of formula (XIIa) is optionally substituted.

In certain embodiments both —R⁴ and —R⁸ of formula (XIIa) are —H.

In certain embodiments —R³ is methyl. In certain embodiments —R³ is —H.

In certain embodiments —R² is —H.

In certain embodiments -L¹- is of formula (XIIa-i)

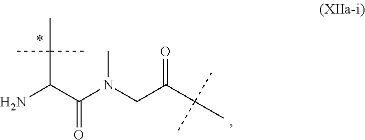

the unmarked dashed line indicates attachment to -D-; and
the dashed line marked with the asterisk indicates attachment to -L²-.

In certain embodiments -L¹- is of formula (XIIa-ii)

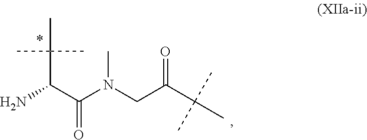

the unmarked dashed line indicates attachment to -D-; and
the dashed line marked with the asterisk indicates attachment to -L²-.

In certain embodiments -L¹- is of formula (XIIa-iii)

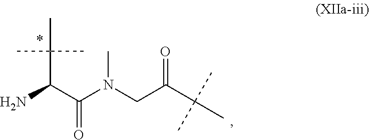

the unmarked dashed line indicates attachment to -D-; and
the dashed line marked with the asterisk indicates attachment to -L²-.

In certain embodiments -L¹- has a structure as disclosed in formula II of WO2022/096636 A1. Accordingly, in certain embodiments the moiety -L¹- is of formula (XIIb):

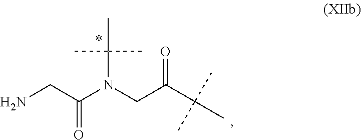

wherein
the unmarked dashed line indicates attachment to -D-; and
the dashed line marked with the asterisk indicates attachment to -L²-.

In certain embodiments -L²- is absent.

In certain embodiments -L²- is a spacer moiety, in particular selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2} which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R⁵R^{y5a}), —S(O)₂N(R^{y5}R^{y5a}), —S(O)N(R⁵R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R⁵R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -L²- is selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R⁵R^{y5a}), —S(O)₂N(R⁵R^{y5a}), —S(O)N(R⁵R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5}) C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R⁵R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—; —R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; each —R^{y2} is independently selected from the group consisting of halogen, and C₁₋₆ alkyl; and each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, —S—, -T- and —C(O)N(R^{y1})—; and which C₁₋₂₀ alkyl chain is optionally substituted with one or more groups independently selected from —OH, -T and —C(O)N(R⁶R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight in the range of from 14 g/mol to 750 g/mol.

In certain embodiments -L²- comprises a moiety selected from

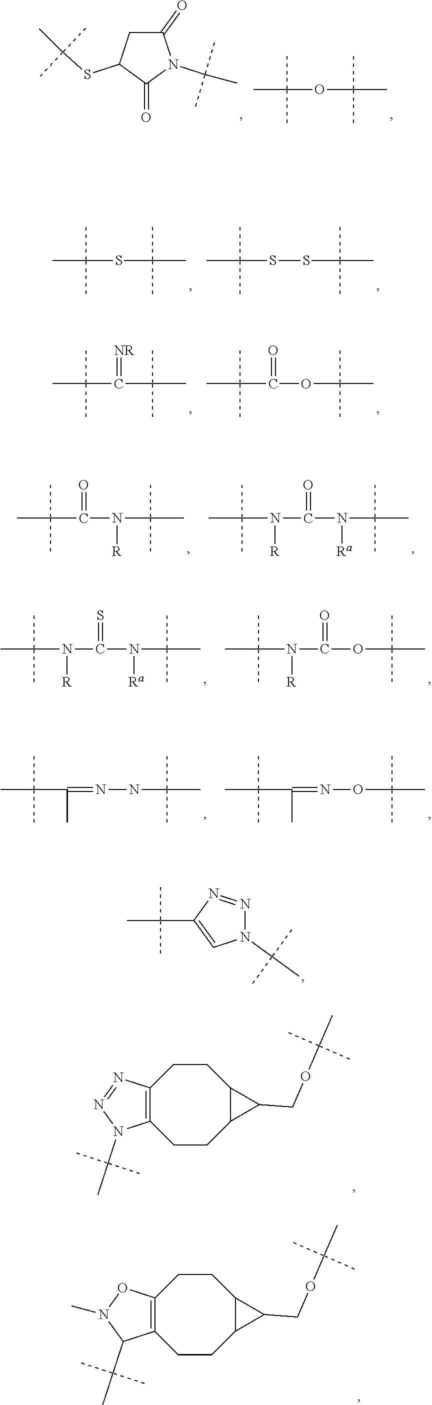

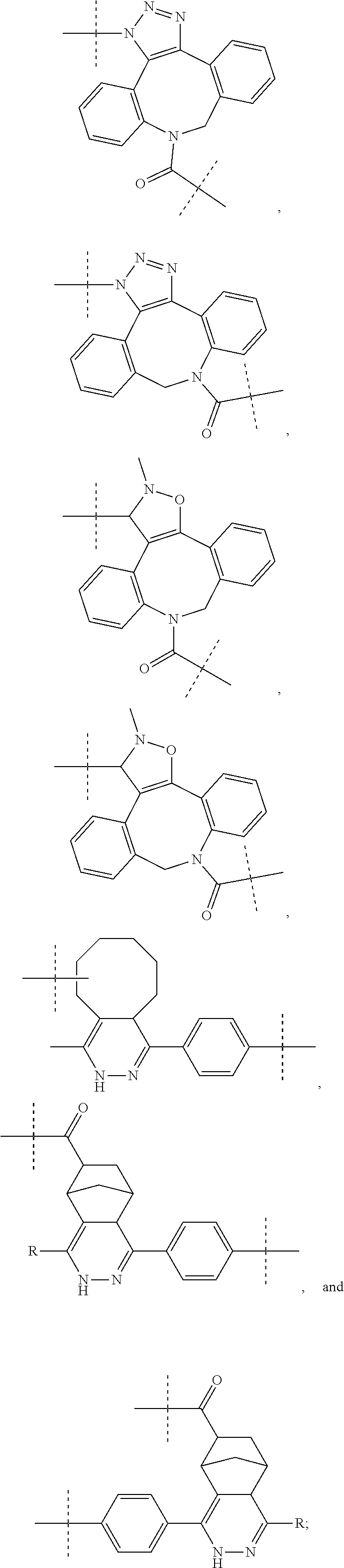

wherein
—R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments -L²- has a chain length of 1 to 20 atoms. In certain embodiments -L²- has a chain length of 2 to 10 atoms.

In certain embodiments -L²- has a chain length of at least 35 atoms. In certain embodiments -L²- has a chain length of at least 50 atoms. In certain embodiments -L²- has a chain length of at least 75 atoms. In certain embodiments -L²- has a chain length of at least 100 atoms. In certain embodiments -L²- has a chain length of at least 150 atoms. In certain embodiments -L²- has a chain length of at least 200 atoms. In certain embodiments -L²- has a chain length of at least 250 atoms. In certain embodiments -L²- has a chain length of at least 300 atoms. In certain embodiments -L²- has a chain length of at most 3000 atoms. In certain embodiments -L²- has a chain length of at most 2500 atoms. In certain embodiments -L²- has a chain length of at most 2000 atoms. In certain embodiments -L²- has a chain length of at most 1500 atoms. In certain embodiments -L²- has a chain length ranging from 35 to 3000 atoms. In certain embodiments -L²- has a chain length ranging from 50 to 2500 atoms. In certain embodiments -L²- has a chain length ranging from 75 to 2000 atoms. In certain embodiments -L²- has a chain length ranging from 100 to 1500 atoms. In certain embodiments -L²- has a chain length ranging from 125 to 1000 atoms.

In certain embodiments -L²- is of formula (XI)

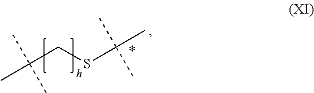

wherein
the unmarked dashed line indicates attachment to -L¹-;
the dashed line marked with the asterisk indicates attachment to -AB¹;
h is an integer selected from the group consisting of 1, 2, 3, 4, 5, 6, 7, 8, 9, 10, 11, 12, 13. 14, 15 and 16.

In certain embodiments -L²- is of formula (XI) and h is 1. In certain embodiments -L²- is of formula (XI) and h is 2. In certain embodiments -L²- is of formula (XI) and h is 3. In certain embodiments -L²- is of formula (XI) and h is 4. In certain embodiments -L²- is of formula (XI) and h is 5. In certain embodiments -L²- is of formula (XI) and h is 6. In certain embodiments -L²- is of formula (XI) and h is 7. In certain embodiments -L²- is of formula (XI) and h is 8. In certain embodiments -L²- is of formula (XI) and h is 9. In certain embodiments -L²- is of formula (XI) and h is 10. In certain embodiments -L²- is of formula (XI) and h is 11. In certain embodiments -L²- is of formula (XI) and h is 12. In certain embodiments -L²- is of formula (XI) and h is 13. In certain embodiments -L²- is of formula (XI) and h is 14. In certain embodiments -L²- is of formula (XI) and h is 15.

In certain embodiments -L²- has a molecular weight of at least 450 Da. In certain embodiments -L²- has a molecular weight of at least 1 kDa. In certain embodiments -L²- has a molecular weight of at least 1.5 kDa. In certain embodiments -L²- has a molecular weight of at least 2 kDa. In certain embodiments -L²- has a molecular weight of at least 2.5 kDa. In certain embodiments -L²- has a molecular weight of at least 3 kDa. In certain embodiments -L²- has a molecular weight of at least 3.5 kDa. In certain embodiments -L²- has a molecular weight of at least 4 kDa. In certain embodiments -L²- has a molecular weight of at least 5 kDa. In certain embodiments -L²- has a maximum molecular weight of 160 kDa. In certain embodiments -L²- has a maximum molecular weight of 120 kDa. In certain embodiments -L²- has a maximum molecular weight of 100 kDa. In certain embodiments -L²- has a maximum molecular weight of 80 kDa. In certain embodiments -L²- has a maximum molecular weight of 70 kDa. In certain embodiments -L²- has a maximum molecular weight of 60 kDa. In certain embodiments -L²- has a maximum molecular weight of 50 kDa. In certain embodiments -L²- has a maximum molecular weight of 40 kDa. In certain embodiments -L²- has a molecular weight of about 450 Da. In certain embodiments -L²- has a molecular weight of about 1 kDa. In certain embodiments -L²- has a molecular weight of about 1.5 kDa. In certain embodiments -L²- has a molecular weight of about 2 kDa. In certain embodiments -L²- has a molecular weight of about 2.5 kDa. In certain embodiments -L²- has a molecular weight of about 3 kDa. In certain embodiments -L²- has a molecular weight of about 3.5 kDa. In certain embodiments -L²- has a molecular weight of about 4 kDa. In certain embodiments -L²- has a molecular weight of about 4.5 kDa. In certain embodiments -L²- has a molecular weight of about 5 kDa. In certain embodiments -L²- has a molecular weight of about 5.5 kDa. In certain embodiments -L²- has a molecular weight of about 6 kDa. In certain embodiments -L²- has a molecular weight of about 6.5 kDa. In certain embodiments -L²- has a molecular weight of about 7 kDa. In certain embodiments -L²- has a molecular weight of about 7.5 kDa. In certain embodiments -L²- has a molecular weight of about 8 kDa. In certain embodiments -L²- has a molecular weight of about 8.5 kDa. In certain embodiments -L²- has a molecular weight of about 9 kDa. In certain embodiments -L²- has a molecular weight of about 9.5 kDa. In certain embodiments -L²- has a molecular weight of about 10 kDa.

In certain embodiments -L²- comprises a polymeric moiety, meaning that it comprises at least one polymer moiety. It is understood that if -L²- comprises one polymer moiety the minimum and maximum molecular weights provided above apply to this one polymer moiety and if -L²- comprises more than one polymer moiety the minimum and maximum molecular weights provided above refer to the minimum and maximum molecular weight of all polymer moieties together.

In certain embodiments the one or more polymer moiety of -L²- has a Flory radius of at least 1.2 nm. In certain embodiments the one or more polymer moiety of -L²- has a Flory radius of at least 1.5 nm. In certain embodiments the one or more polymer moiety of -L²- has a Flory radius of at least 2 nm. In certain embodiments the one or more polymer moiety of -L²- has a Flory radius of at least 2.5 nm. In certain embodiments the one or more polymer moiety of -L²- has a Flory radius of at least 3 nm. In certain embodiments the one or more polymer moiety of -L²- has a Flory radius of at least 3.5 nm. In certain embodiments the one or more polymer moiety of -L²- has a Flory radius of at least 4 nm. In certain embodiments the one or more polymer moiety of -L²- has a Flory radius of at least 4.5 nm. In certain embodiments the one or more polymer moiety of -L²- has a Flory radius of at least 5 nm. In certain embodiments the one or more polymer moiety of -L²- has a Flory radius of no more than 200 nm. In certain embodiments the one or more polymer moiety of -L²- has a Flory radius of no more than 175 nm. In certain embodiments the one or more polymer moiety of -L²- has a Flory radius of no more than 150 nm. In certain embodiments the one or more polymer moiety of -L²- has a Flory radius of no more than 125 nm. In certain embodiments the one or more polymer moiety of -L²- has a Flory radius of no more than 100 nm. In certain embodiments the one or more polymer moiety of -L²- has a Flory radius of no more than 75 nm. In certain embodiments the one or more polymer moiety of -L²- has a Flory radius of no more than 50 nm. In certain embodiments the one or more polymer moiety of -L²- has a Flory radius of no more than 45 nm. In certain embodiments the one or more polymer moiety of -L²- has a Flory radius of no more than 40 nm. In certain embodiments the one or more polymer moiety of -L²- has a Flory radius of no more than 35 nm. In certain embodiments the one or more polymer moiety of -L²- has a Flory radius of no more than 30 nm. In certain embodiments the one or more polymer moiety of -L²- has a Flory radius of about 1.2 nm. In certain embodiments the one or more polymer moiety of -L²- has a Flory radius of about 1.5 nm. In certain embodiments the one or more polymer moiety of -L²- has a Flory radius of about 2 nm. In certain embodiments the one or more polymer moiety of -L²- has a Flory radius of about 2.5 nm. In certain embodiments the one or more polymer moiety of -L²- has a Flory radius of about 3 nm. In certain embodiments the one or more polymer moiety of -L²- has a Flory radius of about 3.5 nm. In certain embodiments the one or more polymer moiety of -L²- has a Flory radius of about 4 nm. In certain embodiments the one or more polymer moiety of -L²- has a Flory radius of about 4.5 nm. In certain embodiments the one or more polymer moiety of -L²- has a Flory radius of about 5 nm. In certain embodiments the one or more polymer moiety of -L²- has a Flory radius of about 5.5 nm. In certain embodiments the one or more polymer moiety of -L²- has a Flory radius of about 6 nm. In certain embodiments the one or more polymer moiety of -L²- has a Flory radius of about 6.5 nm. In certain embodiments the one or more polymer moiety of -L²- has a Flory radius of about 7 nm. In certain embodiments the one or more polymer moiety of -L²- has a Flory radius of about 8.5 nm. In certain embodiments the one or more polymer moiety of -L²- has a Flory radius of about 9 nm. In certain embodiments the one or more polymer moiety of -L²- has a Flory radius of about 10 nm. It is understood that if -L²- comprises one polymer moiety the Flory radius provided above applies to this one polymer moiety and if -L²- comprises more than one polymer moiety the Flory radius provided above refers to the Flory radius of all polymer moieties together.

-L²- comprises one or more polymer moiety, such as polymer moiety selected from the group consisting of poly(2-methacryloyl-oxyethyl phosphonyl cholines), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments -L²- comprises a PEG-based polymer. In certain embodiments -L²- comprises a hyaluronic acid-based polymer. In certain embodiments -L²- comprises a random coil polymer. In certain embodiments -L²- comprises a poly-sarcosine polymer.

In certain embodiments -L²- is of formula (XII)

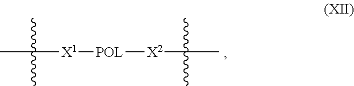

wherein
—X¹—and —X²— are independently selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₂₀ alkyl, C₂₋₂₀ alkenyl, and C₂₋₂₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R⁵R^{y5a}), —S(O)₂N(R⁵R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R⁵R^{y5a}), and C₁₋₆ alkyl; wherein C₁₋₆ alkyl is optionally substituted with one or more halogen, which are the same or different;
each —R^{y3}, —R^{y3a}, —R^{y4}, —R^{y4a}, —R⁵, —R^{y5a} and —R^{y5b} is independently of each other selected from the group consisting of —H, and C₁₋₆ alkyl; wherein C₁₋₆ alkyl is optionally substituted with one or more halogen, which are the same or different; and
-POL- is a polymeric moiety.

In certain embodiments —X¹—and —X²—of formula (XII) are independently selected from the group consisting of C₁₋₂₀ alkyl, C₂₋₂₀ alkenyl, and C₂₋₂₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S—, —N(R^{y3})—, —OC(OR^{y3})(R^{y3a})—, —N(R^{y3})C(O)N(R^{y3a})—, and —OC(O)N(R^{y3})—; each T is independently selected from the group consisting of phenyl, naphthyl, indenyl, indanyl, tetralinyl, C₃₋₁₀ cycloalkyl and 3- to 10-membered heterocyclyl; wherein each T is independently optionally substituted with one or more —R^{y2}, which are the same or different; —R^{y2} is selected from the group consisting of halogen, —CN, oxo (═O), and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y1a} and —R^{y5b} is independently of each other selected from the group consisting of —H, and C₁₋₆ alkyl; wherein C₁₋₆ alkyl is optionally substituted with one or more halogen, which are the same or different.

In certain embodiments -POL- of formula (XII) is of formula (XII-i).

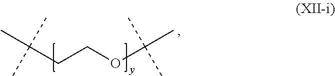

wherein
y is an integer ranging from 2 to 1000.

In certain embodiments y of formula (XII-i) is selected such that the molecular weight of the moiety of formula (XII-i) is about 1 kDa. In certain embodiments y of formula (XII-i) is selected such that the molecular weight of the moiety of formula (XII-i) is about 2 kDa. In certain embodiments y of formula (XII-i) is selected such that the molecular weight of the moiety of formula (XII-i) is about 3 kDa. In certain embodiments y of formula (XII-i) is selected such that the molecular weight of the moiety of formula (XII-i) is about 4 kDa. In certain embodiments y of formula (XII-i) is selected such that the molecular weight of the moiety of formula (XII-i) is about 5 kDa. In certain embodiments y of formula (XII-i) is selected such that the molecular weight of the moiety of formula (XII-i) is about 6 kDa. In certain embodiments y of formula (XII-i) is selected such that the molecular weight of the moiety of formula (XII-i) is about 7 kDa. In certain embodiments y of formula (XII-i) is selected such that the molecular weight of the moiety of formula (XII-i) is about 8 kDa. In certain embodiments y of formula (XII-i) is selected such that the molecular weight of the moiety of formula (XII-i) is about 9 kDa. In certain embodiments y of formula (XII-i) is selected such that the molecular weight of the moiety of formula (XII-i) is about 10 kDa. In certain embodiments y of formula (XII-i) is selected such that the molecular weight of the moiety of formula (XII-i) is about 11 kDa. In certain embodiments y of formula (XII-i) is selected such that the molecular weight of the moiety of formula (XII-i) is about 12 kDa. In certain embodiments y of formula (XII-i) is selected such that the molecular weight of the moiety of formula (XII-i) is about 13 kDa. In certain embodiments y of formula (XII-i) is selected such that the molecular weight of the moiety of formula (XII-i) is about 14 kDa. In certain embodiments y of formula (XII-i) is selected such that the molecular weight of the moiety of formula (XII-i) is about 15 kDa. In certain embodiments y of formula (XII-i) is selected such that the molecular weight of the moiety of formula (XII-i) is about 20 kDa. In certain embodiments y of formula (XII-i) is selected such that the molecular weight of the moiety of formula (XII-i) is about 25 kDa. In certain embodiments y of formula (XII-i) is selected such that the molecular weight of the moiety of formula (XII-i) is about 30 kDa. In certain embodiments y of formula (XII-i) is selected such that the molecular weight of the moiety of formula (XII-i) is about 40 kDa.

In certain embodiments y of formula (XII-i) is 2. In certain embodiments y of formula (XII-i) is about 22. In certain embodiments y of formula (XII-i) is about 45. In certain embodiments y of formula (XII-i) is about 68. In certain embodiments y of formula (XII-i) is about 91. In certain embodiments y of formula (XII-i) is about 113. In certain embodiments y of formula (XII-i) is about 136. In certain embodiments y of formula (XII-i) is about 160. In certain embodiments y of formula (XII-i) is about 182. In certain embodiments y of formula (XII-i) is about 204. In certain embodiments y of formula (XII-i) is about 227. In certain embodiments y of formula (XII-i) is about 250. In certain embodiments y of formula (XII-i) is about 340. In certain embodiments y of formula (XII-i) is about 455. In certain embodiments y of formula (XII-i) is about 910.

In certain embodiments -L²- is of formula (XII-ii)

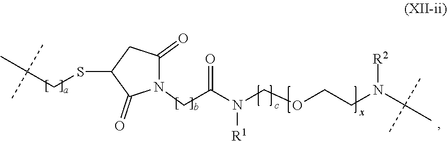

wherein
the dashed line marked with the asterisk indicates attachment to -L¹-;
the unmarked dashed line indicates attachment to -AB¹;
ais 1, 2, 3, 4, 5, 6, 7, 8, 9, 10, 11, 12, 13, 14 or 15;
b is 1, 2, 3, 4, 5, 6, 7, 8, 9, 10, 11, 12, 13, 14 or 15;
c is 1, 2, 3, 4, 5, 6, 7, 8, 9, 10, 11, 12, 13, 14 or 15;
x ranges from 2 to 1000;
—R¹ and —R² are independent of each other selected from the group consisting of —H and C₁₋₆ alkyl.

In certain embodiments x of formula (XII-ii) is 2. In certain embodiments x of formula (XII-ii) is about 22. In certain embodiments x of formula (XII-ii) is about 45. In certain embodiments x of formula (XII-ii) is about 68. In certain embodiments x of formula (XII-ii) is about 91. In certain embodiments x of formula (XII-ii) is about 113. In certain embodiments x of formula (XII-ii) is about 136. In certain embodiments x of formula (XII-ii) is about 160. In certain embodiments x of formula (XII-ii) is about 182. In certain embodiments x of formula (XII-ii) is about 204. In certain embodiments x of formula (XII-ii) is about 227. In certain embodiments x of formula (XII-ii) is about 250. In certain embodiments x of formula (XII-ii) is about 340. In certain embodiments x of formula (XII-ii) is about 455. In certain embodiments x of formula (XII-ii) is about 910.

In certain embodiments a of formula (XII-ii) is 5, b of formula (XII-ii) is 2, c of formula (XII-ii) is 2, and both —R¹ and —R² of formula (XII-ii) are —H.

In certain embodiments the albumin-binding moieties of a compound disclosed herein bind to one albumin, such as via different binding domains. Suitably, the albumin-binding moieties of a compound disclosed herein bind to at least two albumins, such as to two albumins.

In certain embodiments -L¹-L²- is selected from the group consisting

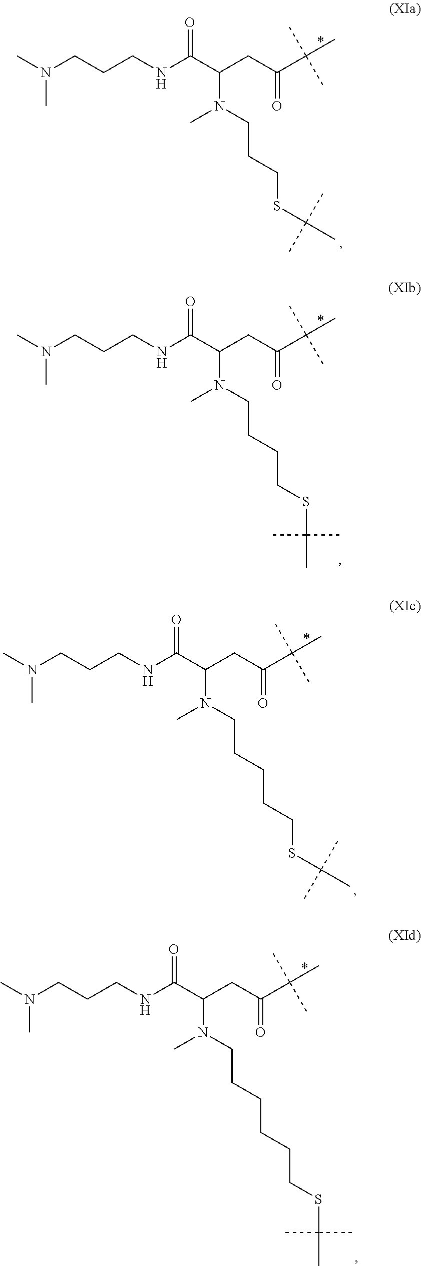

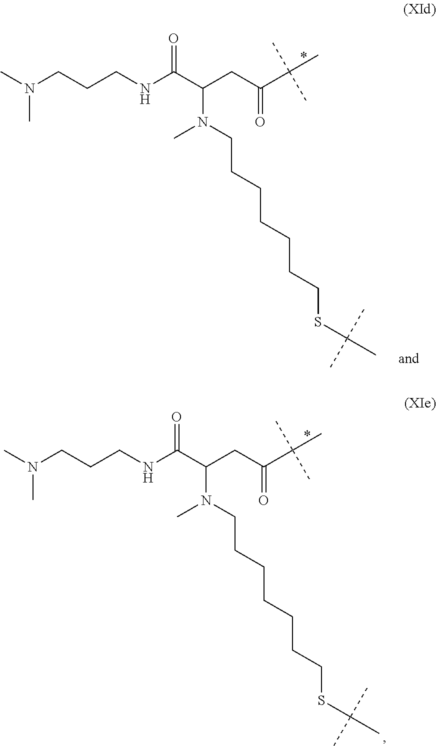

wherein
the dashed line marked with the asterisk indicates attachment to a nitrogen of -D-; and
the unmarked dashed line indicates attachment to -AB¹.

In certain embodiments -L¹-L²- is of formula (XIa). In certain embodiments -L¹-L²- is of formula (XIb). In certain embodiments -L¹-L²- is of formula (XIc). In certain embodiments -L¹-L²- is of formula (XId). In certain embodiments -L¹-L²- is of formula (XIe).

In certain embodiments -L¹-L²- is of formula (XIf).

In certain embodiments -L¹-L²- is selected from the group consisting

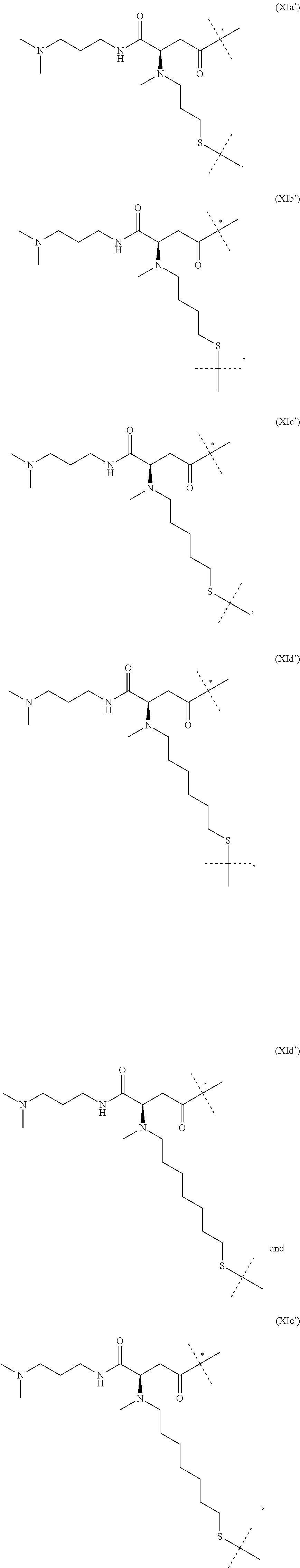

wherein
the dashed line marked with the asterisk indicates attachment to a nitrogen of -D-; and
the unmarked dashed line indicates attachment to -AB¹.

In certain embodiments -L¹-L²- is of formula (Xia′). In certain embodiments -L¹-L²- is of formula (XIb′). In certain embodiments -L¹-L²- is of formula (XIc′). In certain embodiments -L¹-L²- is of formula (XId′). In certain embodiments -L¹-L²- is of formula (XIe′).

In certain embodiments -L¹-L²- is of formula (XIf′).

In certain embodiments -L¹-L²- is selected from the group consisting

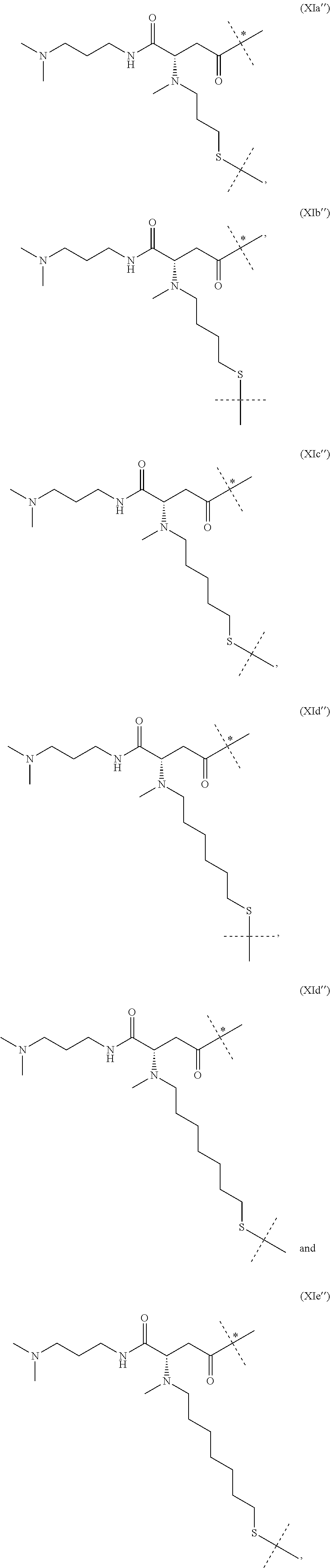

wherein
the dashed line marked with the asterisk indicates attachment to a nitrogen of -D-; and
the unmarked dashed line indicates attachment to -AB¹.

In certain embodiments -L¹-L²- is of formula (Xia″). In certain embodiments -L¹-L²- is of formula (XIb″). In certain embodiments -L¹-L²- is of formula (XIc″). In certain embodiments -L¹-L²- is of formula (XId″). In certain embodiments -L¹-L²- is of formula (XIe″). In certain embodiments -L¹-L²- is of formula (XIf).

In certain embodiments -L¹-L²- is selected from the group consisting

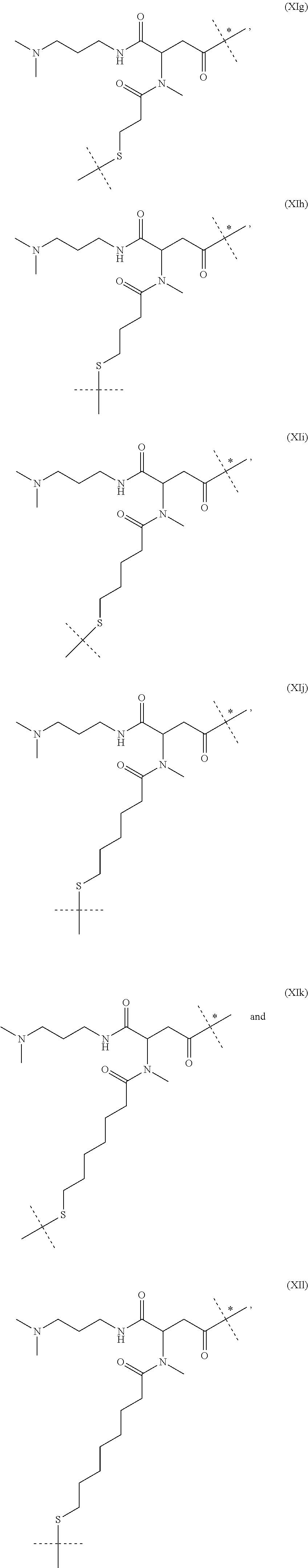

wherein
the dashed line marked with the asterisk indicates attachment to a nitrogen of -D-; and
the unmarked dashed line indicates attachment to -AB¹.

In certain embodiments -L¹-L²- is of formula (XIg). In certain embodiments -L¹-L²- is of formula (XIh). In certain embodiments -L¹-L²- is of formula (XIi). In certain embodiments -L¹-L²- is of formula (XIj). In certain embodiments -L¹-L²- is of formula (XIk). In certain embodiments -L¹-L²- is of formula (XIl).

In certain embodiments -L¹-L²- is selected from the group consisting

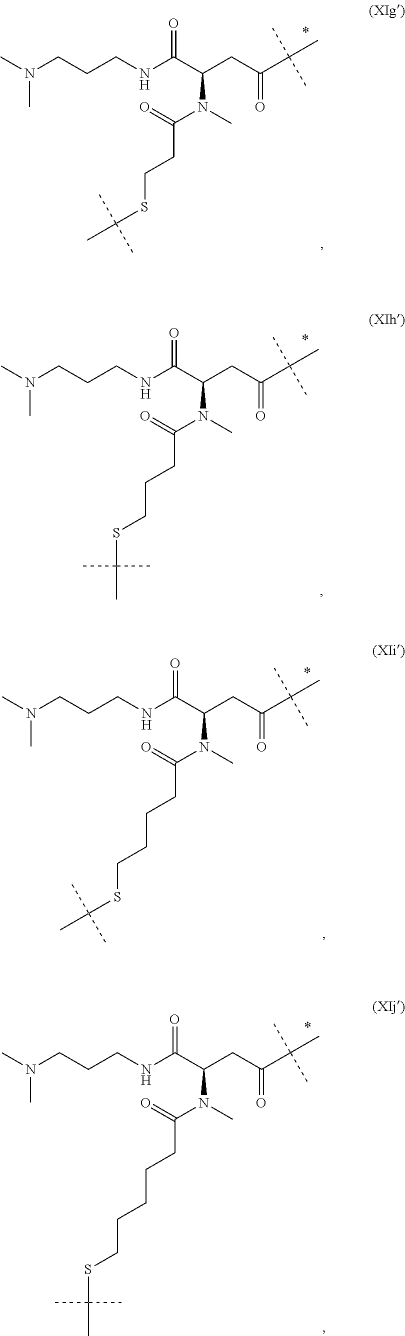

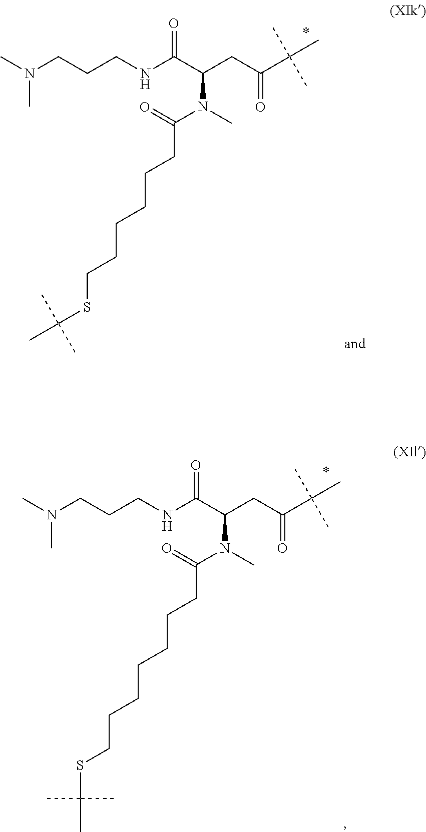

wherein
the dashed line marked with the asterisk indicates attachment to a nitrogen of -D-; and
the unmarked dashed line indicates attachment to -AB¹.

In certain embodiments -L¹-L²- is of formula (XIg′). In certain embodiments -L¹-L²- is of formula (XIh′). In certain embodiments -L¹-L²- is of formula (XIi′). In certain embodiments -L¹-L²- is of formula (XIj′). In certain embodiments -L¹-L²- is of formula (XIk′). In certain embodiments -L¹-L²- is of formula (XIl′).

In certain embodiments -L¹-L²- is selected from the group consisting

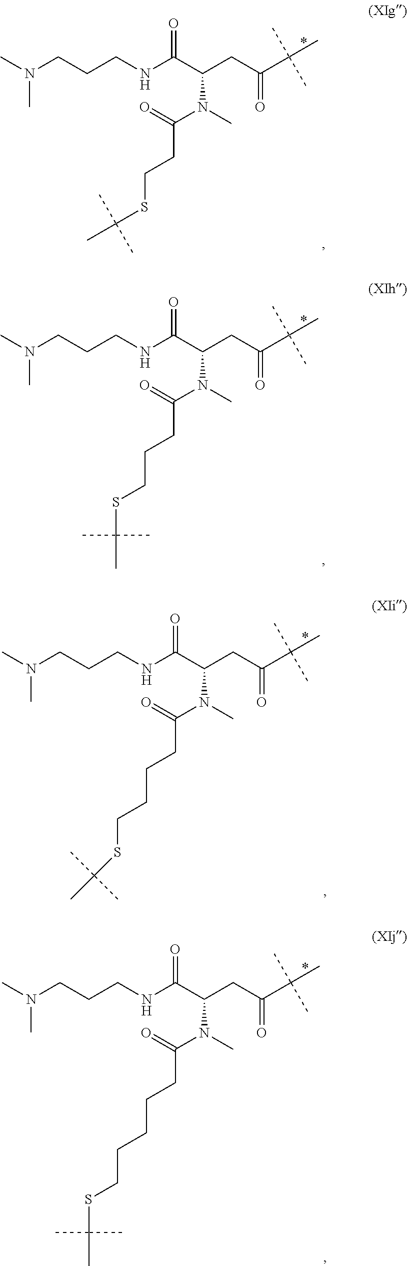

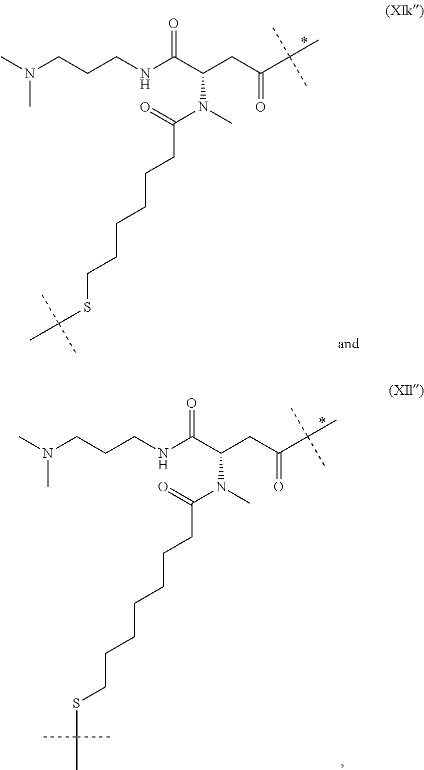

wherein
the dashed line marked with the asterisk indicates attachment to a nitrogen of -D-; and
the unmarked dashed line indicates attachment to -AB¹.

In certain embodiments -L¹-L²- is of formula (XIg″). In certain embodiments -L¹-L²- is of formula (XIh″). In certain embodiments -L¹-L²- is of formula (XIi″). In certain embodiments -L¹-L²- is of formula (XIj″). In certain embodiments -L¹-L²- is of formula (XIk″). In certain embodiments -L¹-L²- is of formula (XIl″).

In certain embodiments the compound is of formula (XIII)

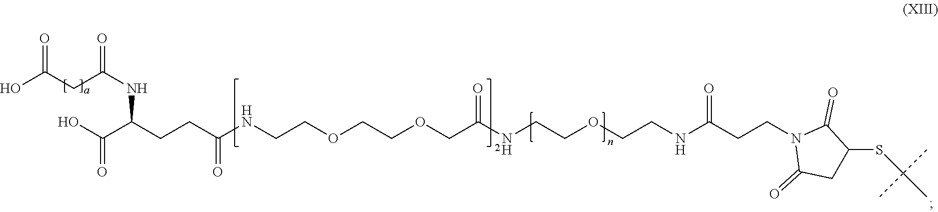

wherein
a is 14, 15, 16, 17, 18, 19, 20, 21 or 22;
n is an integer ranging from 2 to 1000;
the dashed line indicates attachment to a moiety

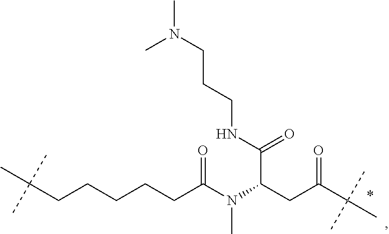

wherein
the unmarked dashed line indicates attachment to the dashed line in formula (XIII); and
the dashed line marked with the asterisk indicates attachment to -D-.

In certain embodiments the compound is of formula (XIII), wherein a is 18, n is 2 and the dashed line marked with the asterisk indicates attachment to the N-terminal amine functional group of semaglutide.

In certain embodiments the compound is of formula (XIII), wherein a is 16, n is 2 and the dashed line marked with the asterisk indicates attachment to the N-terminal amine functional group of semaglutide.

In certain embodiments the compound is of formula (XIII), wherein a is 18, n is approx. 23 and the dashed line marked with the asterisk indicates attachment to the N-terminal amine functional group of semaglutide.

In certain embodiments the compound is of formula (XIII), wherein a is 16, n is approx. 23 and the dashed line marked with the asterisk indicates attachment to the N-terminal amine functional group of semaglutide.

In certain embodiments the compound is of formula (XIII), wherein a is 18, n is approx. 108 and the dashed line marked with the asterisk indicates attachment to the N-terminal amine functional group of semaglutide.

In certain embodiments the compound is of formula (XIII), wherein a is 16, n is approx. 108 and the dashed line marked with the asterisk indicates attachment to the N-terminal amine functional group of semaglutide.

In certain embodiments the distance between a moiety AB¹- and -AB² is such that they are capable of binding to a binding domain of two different albumins or to two different binding domains of the same albumin. In certain embodiments the distance between a moiety AB¹- and -AB² is such that they are capable of binding to a binding domain of two different albumins.

In another aspect the present invention relates to a pharmaceutical composition comprising at least one compound of the present invention or a pharmaceutically acceptable salt thereof and at least one excipient.

In another aspect the present invention relates to a compound of the present invention or a pharmaceutically acceptable salt thereof or a pharmaceutical composition of the present invention for use as a medicament, optionally in combination with one or more additional therapeutically active compounds.

The present invention also relates to a compound or its pharmaceutically acceptable salt thereof or a pharmaceutical composition of the present invention for use as a medicament.

The present invention also relates to a compound or its pharmaceutically acceptable salt thereof or a pharmaceutical composition of the present invention for use in the manufacture of a medicament.

Such medicament may be used in the treatment or prevention of a disease that can be treated or prevented with H-D-AB².

Another aspect of the present invention relates to a compound of the present invention or a pharmaceutically acceptable salt thereof or a pharmaceutical composition comprising said compound of the present invention for use in a method of treating or preventing a disease that can be treated or prevented with H-D-AB² or its pharmaceutically acceptable salt thereof, optionally in combination with one or more additional therapeutically active compounds.

In another aspect the present invention relates to a method of treating a patient having a disease that can be treated or prevented with H-D-AB², wherein the method comprises the step of administering a pharmaceutically acceptable amount of a compound or a pharmaceutically acceptable salt thereof or a pharmaceutical composition of the present invention to a patient in need thereof, optionally in combination with one or more additional therapeutically active compounds.

Administration of a compound, a pharmaceutically acceptable salt thereof or a pharmaceutical composition of the present invention may be via external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion; direct delivery to the brain via implanted device allowing delivery of the invention or the like to brain tissue or brain fluids (e.g., Ommaya Reservoir), direct intracerebroventricular injection or infusion, injection or infusion into brain or brain associated regions, injection into the subchoroidal space, retro-orbital injection and ocular instillation. In certain embodiments the medicament is for subcutaneous injection, which may be done with a pen injector or via a syringe.

A compound, in which -D- or -D-AB² is a GLP-1 receptor agonist moiety or its pharmaceutically acceptable salt thereof or a pharmaceutical composition comprising such compound may be used in the treatment or prevention of a disease selected from the group consisting of (i) all forms of diabetes, (ii) obesity, (iii) non-alcoholic fatty liver disease (NAFLD) and non-alcoholic steatohepatitis (NASH), (iv) cardiovascular disease, (v) neurodegenerative disorders, (vi) chronic kidney disease (CKD), (vii) diabetic kidney disease (DKD), (viii) peripheral arterial disease (PAD), and/or (ix) heart failure (HF).

Exemplary cardiovascular diseases may be selected from the group consisting of syndrome X, atherosclerosis, myocardial infarction, coronary heart disease, reperfusion injury, stroke, cerebral ischemia, an early cardiac or early cardiovascular disease, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (atherosclerosis obliterans), diastolic dysfunction, and/or systolic dysfunction; and reduction of blood pressure, such as reduction of systolic blood pressure.

A compound, in which -D- or -D-AB² is a GLP-1 receptor agonist moiety or its pharmaceutically acceptable salt thereof or a pharmaceutical composition comprising such compound may be used in the treatment or prevention of a disease selected from the group consisting of dyslipidemia and/or diseases where one or more of the following clinical outcomes are the treatment goal: lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein A (apo(A)).

Exemplary neurodegenerative disorders may be selected from the group consisting of Alzheimer's disease and Parkinson's disease.

Exemplary forms of HF may be selected from the group consisting of heart failure with reduced ejection fraction (HFrEF), heart failure with mid-range ejection fraction (HFmrEF) and heart failure with preserved ejection fraction (HFpEF).

A compound, in which -D- or -D-AB² is a GLP-1 receptor agonist moiety or its pharmaceutically acceptable salt thereof or a pharmaceutical composition comprising such compound may be used for the treatment of non-alcoholic fatty liver disease (NAFLD) and non-alcoholic steatohepatitis (NASH).

A compound, in which -D- or -D-AB² is a GLP-1 receptor agonist moiety or its pharmaceutically acceptable salt thereof or a pharmaceutical composition comprising such compound may in certain embodiments be used for the treatment of a disease selected from the group consisting of obesity and eating disorders, where one or more of the following clinical outcomes are the treatment goal: decreasing food intake, increasing energy expenditure, reducing body weight, suppressing appetite, inducing satiety.

A compound, in which -D- or -D-AB² is a GLP-1 receptor agonist moiety or its pharmaceutically acceptable salt thereof or a pharmaceutical composition comprising such compound may also be combined with one or more additional drugs, such as drugs selected from cardiovascular agents, antidiabetic agents, and/or anti-obesity agents. Examples of these pharmacologically active substances are: inotropes, beta adrenergic receptor blockers, HMG-CoA reductase inhibitors, angiotensin II receptor antagonists, angiotensin converting enzyme inhibitors, calcium channel blockers, endothelin antagonists, renin inhibitors, diuretics, aldosterone receptor blockers, endothelin receptor blockers, aldosterone synthase inhibitors, CETP inhibitor, relaxin, PCSK9 inhibitors, BNP and NEP inhibitors, GLP-1 analogues, insulin, sulphonylureas, biguanides, meglitinides, glucosidase inhibitors, glucagon antagonists, DPP-IV inhibitors, SGLT2 inhibitors. The treatment with the compound or pharmaceutically its pharmaceutically acceptable salt or the pharmaceutical composition of the present invention may also be combined with heart surgery.

A compound, in which -D-AB² is a GLP-1 receptor agonist, or its pharmaceutically acceptable salt thereof or a pharmaceutical composition of the present invention may also be administered in combination with a growth hormone, such as a human growth hormone. Such human growth hormone may be in the form of an unmodified drug, such as the human growth hormone of SEQ ID NO:58, or as a conjugate or complex comprising human growth hormone. Suitably, the compound, in which—D-AB² is a GLP-1 receptor agonist, is administered to a patient in a co-treatment with lonapegsomatropin, which has the following structure:

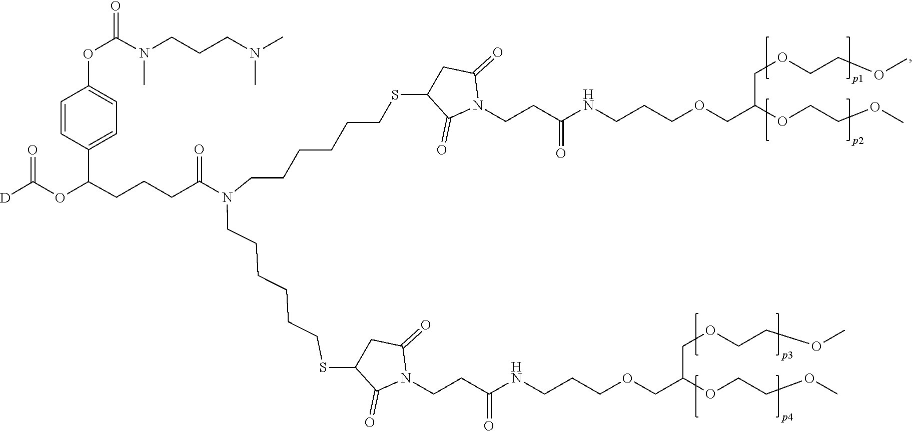

wherein
D is a human growth hormone polypeptide of SEQ ID NO:58 connected to the rest of the molecule through an amine functional group provided by a lysine side chain; and each p1, p2, p3, p4 is independently an integer ranging from 210 to 240.

SEQ ID NO: 58 has the following sequence:
FPTIPLSRLFDNAMLRAHRLHQLAFDTYQEFEEAYIPKEQKYSFLQNPQ
TSLCFSESIPTPSNREETQQKSNLELLRISLLLIQSWLEPVQFLRSVFA
NSLVYGASDSNVYDLLKDLEEGIQTLMGRLEDGSPRTGQIFKQTYSKFD
TNSHNDDALLKNYGLLYCFRKDMDKVETFLRIVQCRSVEGSCGF

In such co-treatment the compound, in which -D-AB² is a GLP-1 receptor agonist, may be administered prior to, at the same time or after administration of the unmodified human growth hormone, the conjugate or complex comprising human growth hormone.

A compound or its pharmaceutically acceptable salt thereof or a pharmaceutical composition of the present invention may be administered, such as via subcutaneous administration, once a week, every two weeks, every three weeks or once a month.

In certain embodiments a compound or its pharmaceutically acceptable salt thereof or a pharmaceutical composition of the present invention may be administered once a week, every two weeks, every three weeks or once a month.

The invention is further described by the following non-limiting items.
1. A compound or a pharmaceutically acceptable salt thereof of formula (Ia) or (Ib)

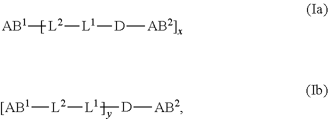

wherein
each -D- is independently a drug moiety;
each -AB¹ and -AB² is independently an albumin-binding moiety;
each -L¹- is independently a linker moiety covalently and reversibly connected to -D-;
each -L²- is independently a spacer or is absent;
x is an integer selected from the group consisting of 1, 2, 3, 4, 5, 6, 7, 8, 9, 10, 11, 12, 13, 14, 15, 16, 17, 18, 19, 20, 21, 22, 23, 24 and 25; and
y is an integer selected from the group consisting of 2, 3, 4 and 5.
2. The compound or pharmaceutically acceptable salt thereof of item 1, wherein the compound is of formula (Ia).
3. The compound or pharmaceutically acceptable salt thereof of item 1 or 2, wherein x is 1.
4. The compound or pharmaceutically acceptable salt thereof of any one of items 1 to 3, wherein -D- or -D-AB² is a GLP-1 receptor agonist moiety.
5. The compound or pharmaceutically acceptable salt thereof of any one of items 1 to 4, wherein -D-AB² is semaglutide.
6. The compound or pharmaceutically acceptable salt thereof of any one of items 1 to 5, wherein -L¹- is of formula (XIIb).
7. The compound or pharmaceutically acceptable salt thereof of any one of items 1 to 5, wherein -L¹- is of formula (XIIa).
8. The compound or pharmaceutically acceptable salt thereof of any one of items 1 to 5 or 7, wherein -L¹- is of formula (XIIa-i).
9. The compound or pharmaceutically acceptable salt thereof of any one of items 1 to 5, 7 or 8, wherein -L¹- is of formula (XIIa-ii).
10. The compound or pharmaceutically acceptable salt thereof of any one of items 1 to 5, 7 or 8, wherein -L¹- is of formula (XIIa-iii).
11. The compound or pharmaceutically acceptable salt thereof of any one of items 1 to 5, wherein -L¹- is of formula (II).
12. The compound or pharmaceutically acceptable salt thereof of item 11, wherein —R¹ and R^{1a} of formula (II) are both —H.
13. The compound or pharmaceutically acceptable salt thereof of item 11, wherein —R¹ of formula (II) is —H and R^{1a} of formula (II) is methyl.
14. The compound or pharmaceutically acceptable salt thereof of item 11, wherein —R¹ and R^{1a} of formula (II) are both methyl.
15. The compound or pharmaceutically acceptable salt thereof of item 11, wherein —R¹ and R^{1a} of formula (II) are both —H.
16. The compound or pharmaceutically acceptable salt thereof of any one of items 11 to 15, wherein —X—of formula (II) is —C(R⁷R^{7a})—.
17. The compound or pharmaceutically acceptable salt thereof of any one of items 11 to 15, wherein —X—of formula (II) is —C(R⁴R^{4a})—,
18. The compound or pharmaceutically acceptable salt thereof of any one of items 11 to 15, wherein —X— is of formula (II) —C(R⁴R^{4a})—C(R⁵R^{5a})—,
19. The compound or pharmaceutically acceptable salt thereof of item 16, wherein —R⁷ of formula (II) is —NR¹⁰—(C═O)—R¹¹.
20. The compound or pharmaceutically acceptable salt thereof of item 16, wherein —R⁷ of formula (II) is —N(R¹⁰R^{10a})
21. The compound or pharmaceutically acceptable salt thereof of item 19 or 20, wherein —R¹⁰ of formula (II) is methyl.
22. The compound or pharmaceutically acceptable salt thereof of item 19 or 20, wherein —R¹⁰ of formula (II) is H.
23. The compound or pharmaceutically acceptable salt thereof of any one of items 20 to 22, wherein —R^{10a} of formula (II) is —H.
24. The compound or pharmaceutically acceptable salt thereof of any one of items 20 to 22, wherein —R^{10a} of formula (II) is methyl.
25. The compound or pharmaceutically acceptable salt thereof of any one of items 16, 19 or 21 to 23, wherein —R¹¹ of formula (II) is —H, which is substituted with -L²-.
26. The compound or pharmaceutically acceptable salt thereof of any one of items 11 to 25, wherein X¹ of formula (II) is C.
27. The compound or pharmaceutically acceptable salt thereof of any one of items 11 to 26, wherein ═X³ of formula (II) is O.
28. The compound or pharmaceutically acceptable salt thereof of any one of items 11 to 27, wherein —X²—of formula (II) is —C(R⁸R^{8a})—C(R⁹R^{9a})—,
29. The compound or pharmaceutically acceptable salt thereof of any one of items 11 to 27, wherein —X²—of formula (II) is —C(R⁸R^{8a})—.
30. The compound or pharmaceutically acceptable salt thereof of item 28 or 29, wherein —R⁸ and —R^{8a} of formula (II) are both —H.
31. The compound or pharmaceutically acceptable salt thereof of item 28 or 30, wherein —R⁹ and —R^{9a} of formula (II) are both —H.
32. The compound or pharmaceutically acceptable salt thereof of any one of items 11 to 31, wherein —R² and —R^{2a} of formula (II) are both —H.
33. The compound or pharmaceutically acceptable salt thereof of any one of items 11 to 31, wherein —R² of formula (II) is methyl and —R^{2a} of formula (II) is —H.
34. The compound or pharmaceutically acceptable salt thereof of any one of items 11 to 31, wherein —R² and —R^{2a} of formula (II) are both methyl.
35. The compound or pharmaceutically acceptable salt thereof of any one of items 11 to 34, wherein —R² and —R^{2a} of formula (II) are both methyl.
36. The compound or pharmaceutically acceptable salt thereof of any one of items 11 to 34, wherein —R² and —R^{2a} of formula (II) are both —H.
37. The compound or pharmaceutically acceptable salt thereof of any one of items 11 to 34, wherein —R² of formula (II) is methyl and —R^{2a} of formula (II) is —H.
38. The compound or pharmaceutically acceptable salt thereof of any one of items 1 to 5, wherein -L¹- is of formula (III).
39. The compound or pharmaceutically acceptable salt thereof of any one of items 1 to 5, wherein -L¹- is of formula (IV).
40. The compound or pharmaceutically acceptable salt thereof of any one of items 1 to 5, wherein -L¹- is of formula (V).
41. The compound or pharmaceutically acceptable salt thereof of any one of items 1 to 5, wherein -L¹- is of formula (VI).
42. The compound or pharmaceutically acceptable salt thereof of any one of items 1 to 5, wherein -L¹- is of formula (VII).
43. The compound or pharmaceutically acceptable salt thereof of any one of items 1 to 5, wherein -L¹- is of formula (VIII).
44. The compound or pharmaceutically acceptable salt thereof of any one of items 1 to 5, wherein -L¹- is of formula (IX).
45. The compound or pharmaceutically acceptable salt thereof of any one of items 1 to 5, wherein -L¹- is of formula (X).
46. The compound or pharmaceutically acceptable salt thereof of any one of items 1 to 45, wherein -L¹- is conjugated to the N-terminal amine functional group of -D-.
47. The compound or pharmaceutically acceptable salt thereof of any one of items 1 to 46, wherein -AB¹ is of formula (A).
48. The compound or pharmaceutically acceptable salt thereof of item 47, wherein —F⁰ of formula (A) is selected from the group consisting of formulas (a-4). (a-5), (a-6), (a-7), (a-8), (a-9), (a-10), (a-11), (a-12), (a-13), (a-14), (a-15), (a-16), (a-17), (a-18), (a, -19), (a-20), (a-21), (a-22), (a-23), (a-24), (a-25), (a-26), (a-27), (a-28), (a-29), (a-30), (a-31), (a-32), (a-33), (a-34), (a-35), (a-36), (a-37), (a-38) and (a-39).
49. The compound or pharmaceutically acceptable salt thereof of item 47 or 48, wherein —F³ of formula (A) is of formula (a-4).
50. The compound or pharmaceutically acceptable salt thereof of item 47 or 48, wherein —F⁰ of formula (A) is of formula (a-5).
51. The compound or pharmaceutically acceptable salt thereof of item 47 or 48, wherein —F⁰ of formula (A) is of formula (a-6).
52. The compound or pharmaceutically acceptable salt thereof of item 47 or 48, wherein —F⁰ of formula (A) is of formula (a-7).
53. The compound or pharmaceutically acceptable salt thereof of item 47 or 48, wherein —F⁰ of formula (A) is of formula (a-8).
54. The compound or pharmaceutically acceptable salt thereof of item 47 or 48, wherein —F of formula (A) is of formula (a-9).
55. The compound or pharmaceutically acceptable salt thereof of item 47 or 48, wherein —F⁰ of formula (A) is of formula (a-10).
56. The compound or pharmaceutically acceptable salt thereof of item 47 or 48, wherein —F⁰ of formula (A) is of formula (a-11).
57. The compound or pharmaceutically acceptable salt thereof of item 47 or 48, wherein —F⁰ of formula (A) is of formula (a-12).
58. The compound or pharmaceutically acceptable salt thereof of item 47 or 48, wherein —F⁰ of formula (A) is of formula (a-13).
59. The compound or pharmaceutically acceptable salt thereof of item 47 or 48, wherein —F⁰ of formula (A) is of formula (a-14).
60. The compound or pharmaceutically acceptable salt thereof of item 47 or 48, wherein —F⁰ of formula (A) is of formula (a-15).
61. The compound or pharmaceutically acceptable salt thereof of item 47 or 48, wherein —F⁰ of formula (A) is of formula (a-16).
62. The compound or pharmaceutically acceptable salt thereof of item 47 or 48, wherein —F⁰ of formula (A) is of formula (a-17).
63. The compound or pharmaceutically acceptable salt thereof of item 47 or 48, wherein —F⁰ of formula (A) is of formula (a-18).
64. The compound or pharmaceutically acceptable salt thereof of item 47 or 48, wherein —F⁰ of formula (A) is of formula (a-19).
65. The compound or pharmaceutically acceptable salt thereof of item 47 or 48, wherein —F⁰ of formula (A) is of formula (a-20).
66. The compound or pharmaceutically acceptable salt thereof of item 47 or 48, wherein —F⁰ of formula (A) is of formula (a-21).
67. The compound or pharmaceutically acceptable salt thereof of item 47 or 48, wherein —F⁰ of formula (A) is of formula (a-22).
68. The compound or pharmaceutically acceptable salt thereof of item 47 or 48, wherein —F⁰ of formula (A) is of formula (a-23).
69. The compound or pharmaceutically acceptable salt thereof of item 47 or 48, wherein —F⁰ of formula (A) is of formula (a-24).
70. The compound or pharmaceutically acceptable salt thereof of item 47 or 48, wherein —F⁰ of formula (A) is of formula (a-25).
71. The compound or pharmaceutically acceptable salt thereof of item 47 or 48, wherein —F⁰ of formula (A) is of formula (a-26).
72. The compound or pharmaceutically acceptable salt thereof of item 47 or 48, wherein —F⁰ of formula (A) is of formula (a-27).
73. The compound or pharmaceutically acceptable salt thereof of item 47 or 48, wherein —F⁰ of formula (A) is of formula (a-28).
74. The compound or pharmaceutically acceptable salt thereof of item 47 or 48, wherein —F⁰ of formula (A) is of formula (a-29).
75. The compound or pharmaceutically acceptable salt thereof of item 47 or 48, wherein —F⁰ of formula (A) is of formula (a-30).
76. The compound or pharmaceutically acceptable salt thereof of item 47 or 48, wherein —F⁰ of formula (A) is of formula (a-31).
77. The compound or pharmaceutically acceptable salt thereof of item 47 or 48, wherein —F⁰ of formula (A) is of formula (a-32).
78. The compound or pharmaceutically acceptable salt thereof of item 47 or 48, wherein —F⁰ of formula (A) is of formula (a-33).
79. The compound or pharmaceutically acceptable salt thereof of item 47 or 48, wherein —F⁰ of formula (A) is of formula (a-34).
80. The compound or pharmaceutically acceptable salt thereof of item 47 or 48, wherein —F⁰ of formula (A) is of formula (a-35).
81. The compound or pharmaceutically acceptable salt thereof of item 47 or 48, wherein —F⁰ of formula (A) is of formula (a-36).
82. The compound or pharmaceutically acceptable salt thereof of item 47 or 48, wherein —F⁰ of formula (A) is of formula (a-37).
83. The compound or pharmaceutically acceptable salt thereof of item 47 or 48, wherein —F⁰ of formula (A) is of formula (a-38).
84. The compound or pharmaceutically acceptable salt thereof of item 47 or 48, wherein —F⁰ of formula (A) is of formula (a-39).
85. The compound or pharmaceutically acceptable salt thereof of any one of items 1 to 84, wherein -L^{A}- of formula (A) is absent.
86. The compound or pharmaceutically acceptable salt thereof of any one of items 47 to 84, wherein -L^{A}- of formula (A) is selected from the group consisting of formulas (a-40), (a-41), (a-42), (a-43), (a-44), (a-45), (a-46), (a-47), (a-48), (a-49), (a-50), (a-51), (a-52), (a-53), (a-54), (a-55), (a-56), (a-57), (a-58), (a-59), (a-60), (a-61), (a-62), (a-63), (a-64), (a-65), (a-66), (a-67), (a-68), (a-69), (a-70), (a-71), (a-72), (a-73), (a-74), (a-75), (a-76), (a-77), (a-78), (a-79), (a-80), (a-81) and (a-82).
87. The compound or pharmaceutically acceptable salt thereof of any one of items 47 to 84 or 86, wherein -L^{A}- of formula (A) is of formula (a-40).
88. The compound or pharmaceutically acceptable salt thereof of any one of items 47 to 84 or 86, wherein -L^{A}- of formula (A) is of formula (a-41).
89. The compound or pharmaceutically acceptable salt thereof of any one of items 47 to 84 or 86, wherein -L^{A}- of formula (A) is of formula (a-42).
90. The compound or pharmaceutically acceptable salt thereof of any one of items 47 to 84 or 86, wherein -L^{A}- of formula (A) is of formula (a-43).
91. The compound or pharmaceutically acceptable salt thereof of any one of items 47 to 84 or 86, wherein -L^{A}- of formula (A) is of formula (a-44).
92. The compound or pharmaceutically acceptable salt thereof of any one of items 47 to 84 or 86, wherein -L^{A}- of formula (A) is of formula (a-45).
93. The compound or pharmaceutically acceptable salt thereof of any one of items 47 to 84 or 86, wherein -L^{A}- of formula (A) is of formula (a-46).
94. The compound or pharmaceutically acceptable salt thereof of any one of items 47 to 84 or 86, wherein -L^{A}- of formula (A) is of formula (a-47).
95. The compound or pharmaceutically acceptable salt thereof of any one of items 47 to 84 or 86, wherein -L^{A}- of formula (A) is of formula (a-48).
96. The compound or pharmaceutically acceptable salt thereof of any one of items 47 to 84 or 86, wherein -L^{A}- of formula (A) is of formula (a-49).
97. The compound or pharmaceutically acceptable salt thereof of any one of items 47 to 84 or 86, wherein -L^{A}- of formula (A) is of formula (a-50).
98. The compound or pharmaceutically acceptable salt thereof of any one of items 47 to 84 or 86, wherein -L^{A}- of formula (A) is of formula (a-51).
99. The compound or pharmaceutically acceptable salt thereof of any one of items 47 to 84 or 86, wherein -L^{A}- of formula (A) is of formula (a-52).
100. The compound or pharmaceutically acceptable salt thereof of any one of items 47 to 84 or 86, wherein -L^{A}- of formula (A) is of formula (a-53).
101. The compound or pharmaceutically acceptable salt thereof of any one of items 47 to 84 or 86, wherein -L^{A}- of formula (A) is of formula (a-54).
102. The compound or pharmaceutically acceptable salt thereof of any one of items 47 to 84 or 86, wherein -L^{A}- of formula (A) is of formula (a-55).
103. The compound or pharmaceutically acceptable salt thereof of any one of items 47 to 84 or 86, wherein -L^{A}- of formula (A) is of formula (a-56).
104. The compound or pharmaceutically acceptable salt thereof of any one of items 47 to 84 or 86, wherein -L^{A}- of formula (A) is of formula (a-57).
105. The compound or pharmaceutically acceptable salt thereof of any one of items 47 to 84 or 86, wherein -L^{A}- of formula (A) is of formula (a-58).
106. The compound or pharmaceutically acceptable salt thereof of any one of items 47 to 84 or 86, wherein -L^{A}- of formula (A) is of formula (a-59).
107. The compound or pharmaceutically acceptable salt thereof of any one of items 47 to 84 or 86, wherein -L^{A}- of formula (A) is of formula (a-60).
108. The compound or pharmaceutically acceptable salt thereof of any one of items 47 to 84 or 86, wherein -L^{A}- of formula (A) is of formula (a-61).
109. The compound or pharmaceutically acceptable salt thereof of any one of items 47 to 84 or 86, wherein -L^{A}- of formula (A) is of formula (a-62).
110. The compound or pharmaceutically acceptable salt thereof of any one of items 47 to 84 or 86, wherein -L^{A}- of formula (A) is of formula (a-63).
111. The compound or pharmaceutically acceptable salt thereof of any one of items 47 to 84 or 86, wherein -L^{A}- of formula (A) is of formula (a-64).
112. The compound or pharmaceutically acceptable salt thereof of any one of items 47 to 84 or 86, wherein -L^{A}- of formula (A) is of formula (a-65).
113. The compound or pharmaceutically acceptable salt thereof of any one of items 47 to 84 or 86, wherein -L^{A}- of formula (A) is of formula (a-66).
114. The compound or pharmaceutically acceptable salt thereof of any one of items 47 to 84 or 86, wherein -L^{A}- of formula (A) is of formula (a-67).
115. The compound or pharmaceutically acceptable salt thereof of any one of items 47 to 84 or 86, wherein -L^{A}- of formula (A) is of formula (a-68).
116. The compound or pharmaceutically acceptable salt thereof of any one of items 47 to 84 or 86, wherein -L^{A}- of formula (A) is of formula (a-69).
117. The compound or pharmaceutically acceptable salt thereof of any one of items 47 to 84 or 86, wherein -L^{A}- of formula (A) is of formula (a-70).
118. The compound or pharmaceutically acceptable salt thereof of any one of items 47 to 84 or 86, wherein -L^{A}- of formula (A) is of formula (a-71).
119. The compound or pharmaceutically acceptable salt thereof of any one of items 47 to 84 or 86, wherein -L^{A}- of formula (A) is of formula (a-72).
120. The compound or pharmaceutically acceptable salt thereof of any one of items 47 to 84 or 86, wherein -L^{A}- of formula (A) is of formula (a-73).
121. The compound or pharmaceutically acceptable salt thereof of any one of items 47 to 84 or 86, wherein -L^{A}- of formula (A) is of formula (a-74).
122. The compound or pharmaceutically acceptable salt thereof of any one of items 47 to 84 or 86, wherein -L^{A}- of formula (A) is of formula (a-75).
123. The compound or pharmaceutically acceptable salt thereof of any one of items 47 to 84 or 86, wherein -L^{A}- of formula (A) is of formula (a-76).
124. The compound or pharmaceutically acceptable salt thereof of any one of items 47 to 84 or 86, wherein -L^{A}- of formula (A) is of formula (a-77).
125. The compound or pharmaceutically acceptable salt thereof of any one of items 47 to 84 or 86, wherein -L^{A}- of formula (A) is of formula (a-78).
126. The compound or pharmaceutically acceptable salt thereof of any one of items 47 to 84 or 86, wherein -L^{A}- of formula (A) is of formula (a-79).
127. The compound or pharmaceutically acceptable salt thereof of any one of items 47 to 84 or 86, wherein -L^{A}- of formula (A) is of formula (a-80).
128. The compound or pharmaceutically acceptable salt thereof of any one of items 47 to 84 or 86, wherein -L^{A}- of formula (A) is of formula (a-81).
129. The compound or pharmaceutically acceptable salt thereof of any one of items 47 to 84 or 86, wherein -L^{A}- of formula (A) is of formula (a-82).
130. The compound or pharmaceutically acceptable salt thereof of any one of items 47 to 129, wherein -L^{B}- of formula (A) is absent.
131. The compound or pharmaceutically acceptable salt thereof of any one of items 47 to 129, wherein -L^{B}- of formula (A) is selected from the group consisting of formulas (a-83), (a-84), (a-85), (a-86), (a-87), (a-88), (a-89), (a-90), (a-91), (a-92), (a-93) and (a-94).
132. The compound or pharmaceutically acceptable salt thereof of any one of items 47 to 129 or 131, wherein -L^{B}- of formula (A) is of formula (a-83).
133. The compound or pharmaceutically acceptable salt thereof of any one of items 47 to 129 or 131, wherein -L^{B}- of formula (A) is of formula (a-84).
134. The compound or pharmaceutically acceptable salt thereof of item 133, wherein q of formula (a-84) is an integer ranging from 3 to 45.
135. The compound or pharmaceutically acceptable salt thereof of item 133 or 134, wherein q of formula (a-84) is an integer ranging from 4 to 40.
136. The compound or pharmaceutically acceptable salt thereof of any one of items 133 to 135, wherein q of formula (a-84) is an inter ranging from 5 to 35.
137. The compound or pharmaceutically acceptable salt thereof of any one of items 133 to 136, wherein q of formula (a-84) is an inter ranging from 6 to 30.
138. The compound or pharmaceutically acceptable salt thereof of any one of items 133 to 137, wherein q of formula (a-84) is an inter ranging from 7 to 25.
139. The compound or pharmaceutically acceptable salt thereof of any one of items 133 to 138, wherein q of formula (a-84) is an inter ranging from 10 to 20.
140. The compound or pharmaceutically acceptable salt thereof of any one of items 133 to 138, wherein q of formula (a-84) is 23.
141. The compound or pharmaceutically acceptable salt thereof of any one of items 47 to 129 or 131, wherein -L^{B}- of formula (A) is of formula (a-85).
142. The compound or pharmaceutically acceptable salt thereof of any one of items 47 to 129 or 131, wherein -L^{B}- of formula (A) is of formula (a-86).
143. The compound or pharmaceutically acceptable salt thereof of any one of items 47 to 129 or 131, wherein -L^{B}- of formula (A) is of formula (a-87).
144. The compound or pharmaceutically acceptable salt thereof of any one of items 47 to 129 or 131, wherein -L^{B}- of formula (A) is of formula (a-88).
145. The compound or pharmaceutically acceptable salt thereof of any one of items 47 to 129 or 131, wherein -L^{B}- of formula (A) is of formula (a-89).
146. The compound or pharmaceutically acceptable salt thereof of any one of items 47 to 129 or 131, wherein -L^{B}- of formula (A) is of formula (a-90).
147. The compound or pharmaceutically acceptable salt thereof of any one of items 47 to 129 or 131, wherein -L^{B}- of formula (A) is of formula (a-91).
148. The compound or pharmaceutically acceptable salt thereof of any one of items 47 to 129 or 131, wherein -L^{B}- of formula (A) is of formula (a-92).
149. The compound or pharmaceutically acceptable salt thereof of any one of items 47 to 129 or 131, wherein -L^{B}- of formula (A) is of formula (a-93).
150. The compound or pharmaceutically acceptable salt thereof of any one of items 47 to 129 or 131, wherein -L^{B}- of formula (A) is of formula (a-84).
151. The compound or pharmaceutically acceptable salt thereof of any one of items 1 to 47, wherein -AB¹ is of formula (i).
152. The compound or pharmaceutically acceptable salt thereof of item 151, wherein n of formula (i) is 14.
153. The compound or pharmaceutically acceptable salt thereof of item 151, wherein n of formula (i) is 15.
154. The compound or pharmaceutically acceptable salt thereof of item 151, wherein n of formula (i) is 16.
155. The compound or pharmaceutically acceptable salt thereof of item 151, wherein n of formula (i) is 17.
156. The compound or pharmaceutically acceptable salt thereof of item 151, wherein n of formula (i) is 18.
157. The compound or pharmaceutically acceptable salt thereof of item 151, wherein n of formula (i) is 19.
158. The compound or pharmaceutically acceptable salt thereof of item 151, wherein n of formula (i) is 20.
159. The compound or pharmaceutically acceptable salt thereof of item 151, wherein n of formula (i) is 21.
160. The compound or pharmaceutically acceptable salt thereof of item 151, wherein n of formula (i) is 22.
161. The compound or pharmaceutically acceptable salt thereof of any one of items 151 to 160, wherein the stereocenter marked with the asterisk is in R-configuration.
162. The compound or pharmaceutically acceptable salt thereof of any one of items 151 to 160, wherein the stereocenter marked with the asterisk is in S-configuration.
163. The compound or pharmaceutically acceptable salt thereof of any one of items 1 to 46, wherein -AB¹ is of formula

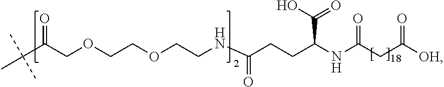

wherein the dashed line indicates attachment to -L²-.
164. The compound or pharmaceutically acceptable salt thereof of any one of items 1 to 46, wherein -AB is of formula

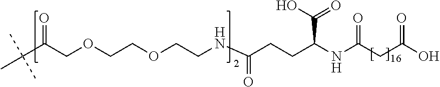

wherein the dashed line indicates attachment to -L²-.
165. The compound or pharmaceutically acceptable salt thereof of any one of items 1 to 164, wherein -L²- has a chain length of at least 35 atoms.
166. The compound or pharmaceutically acceptable salt thereof of any one of items 1 to 165, wherein -L²- has a chain length of at least 50 atoms.
167. The compound or pharmaceutically acceptable salt thereof of any one of items 1 to 166, wherein -L²- has a chain length of at least 75 atoms.
168. The compound or pharmaceutically acceptable salt thereof of any one of items 1 to 167, wherein -L²- has a chain length of at least 100 atoms.
169. The compound or pharmaceutically acceptable salt thereof of any one of items 1 to 168, wherein -L²- has a chain length of at least 150 atoms.
170. The compound or pharmaceutically acceptable salt thereof of any one of items 1 to 169, wherein -L²- has a chain length of at least 200 atoms.
171. The compound or pharmaceutically acceptable salt thereof of any one of items 1 to 170, wherein -L²- has a chain length of at least 250 atoms.
172. The compound or pharmaceutically acceptable salt thereof of any one of items 1 to 171, wherein -L²- has a chain length of at least 300 atoms.
173. The compound or pharmaceutically acceptable salt thereof of any one of items 1 to 172, wherein -L²- has a chain length of at most 3000 atoms.
174. The compound or pharmaceutically acceptable salt thereof of any one of items 1 to 173, wherein -L²- has a chain length of at most 2500 atoms.
175. The compound or pharmaceutically acceptable salt thereof of any one of items 1 to 174, wherein -L²- has a chain length of at most 2000 atoms.
176. The compound or pharmaceutically acceptable salt thereof of any one of items 1 to 175, wherein -L²- has a chain length of at most 1500 atoms.
177. The compound or pharmaceutically acceptable salt thereof of any one of items 1 to 165, wherein -L²- has a chain length ranging from 35 to 3000 atoms.
178. The compound or pharmaceutically acceptable salt thereof of any one of items 1 to 165 or 34, wherein -L²- has a chain length ranging from 50 to 2500 atoms.
179. The compound or pharmaceutically acceptable salt thereof of any one of items 1 to 165, 177 or 178, wherein -L²- has a chain length ranging from 75 to 2000 atoms.
180. The compound or pharmaceutically acceptable salt thereof of any one of items 1 to 165 or 177 to 179, wherein -L²- has a chain length ranging from 100 to 1500 atoms.
181. The compound or pharmaceutically acceptable salt thereof of any one of items 1 to 165 or 177 to 180, wherein -L²- has a chain length ranging from 125 to 1000 atoms.
182. The compound or pharmaceutically acceptable salt thereof of any one of items 1 to 164, wherein -L²- has a molecular weight of at least 450 Da.
183. The compound or pharmaceutically acceptable salt thereof of any one of items 1 to 164 or 182, wherein -L²- has a molecular weight of at least 1 kDa.
184. The compound or pharmaceutically acceptable salt thereof of any one of items 1 to 164, 182 or 183, wherein -L²- has a molecular weight of at least 1.5 kDa.
185. The compound or pharmaceutically acceptable salt thereof of any one of items 1 to 164 or 182 to 184, wherein -L²- has a molecular weight of at least 2 kDa.
186. The compound or pharmaceutically acceptable salt thereof of any one of items 1 to 164 or 182 to 185, wherein -L²- has a molecular weight of at least 2.5 kDa.
187. The compound or pharmaceutically acceptable salt thereof of any one of items 1 to 164 or 182 to 186, wherein -L²- has a molecular weight of at least 3 kDa.
188. The compound or pharmaceutically acceptable salt thereof of any one of items 1 to 164 or 182 to 187, wherein -L²- has a molecular weight of at least 3.5 kDa.
189. The compound or pharmaceutically acceptable salt thereof of any one of items 1 to 164 or 182 to 188, wherein -L²- has a molecular weight of at least 4 kDa.
190. The compound or pharmaceutically acceptable salt thereof of any one of items 1 to 164 or 182 to 189 wherein -L²- has a molecular weight of at least 4.5 kDa.
191. The compound or pharmaceutically acceptable salt thereof of any one of items 1 to 164 or 182 to 190, wherein -L²- has a molecular weight of at least 5 kDa.
192. The compound or pharmaceutically acceptable salt thereof of any one of items 1 to 164 or 182 to 191, wherein -L²- has a molecular weight of at most 160 kDa.
193. The compound or pharmaceutically acceptable salt thereof of any one of items 1 to 164 or 182 to 192, wherein -L²- has a molecular weight of at most 120 kDa.
194. The compound or pharmaceutically acceptable salt thereof of any one of items 1 to 164 or 182 to 193, wherein -L²- has a molecular weight of at most 100 kDa.
195. The compound or pharmaceutically acceptable salt thereof of any one of items 1 to 164 or 182 to 194, wherein -L²- has a molecular weight of at most 80 kDa.
196. The compound or pharmaceutically acceptable salt thereof of any one of items 1 to 164 or 182 to 195, wherein -L²- has a molecular weight of at most 70 kDa.
197. The compound or pharmaceutically acceptable salt thereof of any one of items 1 to 164 or 182 to 196, wherein -L²- has a molecular weight of at most 60 kDa.
198. The compound or pharmaceutically acceptable salt thereof of any one of items 1 to 164 or 182 to 197, wherein -L²- has a molecular weight of at most 50 kDa.
199. The compound or pharmaceutically acceptable salt thereof of any one of items 1 to 164 or 182 to 198, wherein -L²- has a molecular weight of at most 40 kDa.
200. The compound or pharmaceutically acceptable salt thereof of any one of items 1 to 164, wherein -L²- has a molecular weight of about 450 Da.
201. The compound or pharmaceutically acceptable salt thereof of any one of items 1 to 164, wherein -L²- has a molecular weight of about 1 kDa.
202. The compound or pharmaceutically acceptable salt thereof of any one of items 1 to 164, wherein -L²- has a molecular weight of about 1.5 kDa.
203. The compound or pharmaceutically acceptable salt thereof of any one of items 1 to 164, wherein -L²- has a molecular weight of about 2 kDa.
204. The compound or pharmaceutically acceptable salt thereof of any one of items 1 to 164, wherein -L²- has a molecular weight of about 2.5 kDa.
205. The compound or pharmaceutically acceptable salt thereof of any one of items 1 to 164, wherein -L²- has a molecular weight of about 3 kDa.
206. The compound or pharmaceutically acceptable salt thereof of any one of items 1 to 164, wherein -L²- has a molecular weight of about 3.5 kDa.
207. The compound or pharmaceutically acceptable salt thereof of any one of items 1 to 164, wherein -L²- has a molecular weight of about 4 kDa.
208. The compound or pharmaceutically acceptable salt thereof of any one of items 1 to 164, wherein -L²- has a molecular weight of about 4.5 kDa.
209. The compound or pharmaceutically acceptable salt thereof of any one of items 1 to 164, wherein -L²- has a molecular weight of about 5 kDa.
210. The compound or pharmaceutically acceptable salt thereof of any one of items 1 to 164, wherein -L²- has a molecular weight of about 5.5 kDa.
211. The compound or pharmaceutically acceptable salt thereof of any one of items 1 to 164, wherein -L²- has a molecular weight of about 6 kDa.
212. The compound or pharmaceutically acceptable salt thereof of any one of items 1 to 164, wherein -L²- has a molecular weight of about 6.5 kDa.
213. The compound or pharmaceutically acceptable salt thereof of any one of items 1 to 164, wherein -L²- has a molecular weight of about 7 kDa.
214. The compound or pharmaceutically acceptable salt thereof of any one of items 1 to 164, wherein -L²- has a molecular weight of about 7.5 kDa.
215. The compound or pharmaceutically acceptable salt thereof of any one of items 1 to 164, wherein -L²- has a molecular weight of about 8 kDa.
216. The compound or pharmaceutically acceptable salt thereof of any one of items 1 to 164, wherein -L²- has a molecular weight of about 8.5 kDa.
217. The compound or pharmaceutically acceptable salt thereof of any one of items 1 to 164, wherein -L²- has a molecular weight of about 9 kDa.
218. The compound or pharmaceutically acceptable salt thereof of any one of items 1 to 164, wherein -L²- has a molecular weight of about 9.5 kDa.
219. The compound or pharmaceutically acceptable salt thereof of any one of items 1 to 164, wherein -L²- has a molecular weight of about 10 kDa.
220. The compound or pharmaceutically acceptable salt thereof of any one of items 1 to 219, wherein -L²- comprises a polymeric moiety.
221. The compound or pharmaceutically acceptable salt thereof of item 220, wherein the polymeric moiety comprises one or more polymer moiety.
222. The compound or pharmaceutically acceptable salt thereof of item 221, wherein the one or more polymer moiety is selected from the group consisting of poly(2-methacryloyl-oxyethyl phosphonyl cholines), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof
223. The compound or pharmaceutically acceptable salt thereof of item 221 or 222 wherein the one or more polymer moiety is a PEG-based polymer.
224. The compound or pharmaceutically acceptable salt thereof of item 221 or 222, wherein the one or more polymer moiety is a hyaluronic acid-based polymer.
225. The compound or pharmaceutically acceptable salt thereof of item 221 or 222, wherein the one or more polymer moiety comprises a random coil peptide or protein polymer.
226. The compound or pharmaceutically acceptable salt thereof of item 221 or 222, wherein the one or more polymer moiety is a poly-sarcosine-based polymer.
227. The compound or pharmaceutically acceptable salt thereof of any one of items 220 to 226, wherein -L²- has a Flory radius of at least 1.2 nm.
228. The compound or pharmaceutically acceptable salt thereof of any one of items 220 to 227, wherein -L²- has a Flory radius of at least 1.5 nm.
229. The compound or pharmaceutically acceptable salt thereof of any one of items 220 to 228, wherein -L²- has a Flory radius of at least 2 nm.
230. The compound or pharmaceutically acceptable salt thereof of any one of items 220 to 86229 wherein -L²- has a Flory radius of at least 2.5 nm.
231. The compound or pharmaceutically acceptable salt thereof of any one of items 220 to 230, wherein -L²- has a Flory radius of at least 3 nm.
232. The compound or pharmaceutically acceptable salt thereof of any one of items 220 to 231, wherein -L²- has a Flory radius of at least 3.5 nm.
233. The compound or pharmaceutically acceptable salt thereof of any one of items 220 to 232, wherein -L²- has a Flory radius of at least 4 nm.
234. The compound or pharmaceutically acceptable salt thereof of any one of items 220 to 233, wherein -L²- has a Flory radius of at least 4.5 nm.
235. The compound or pharmaceutically acceptable salt thereof of any one of items 220 to 234, wherein -L²- has a Flory radius of at least 5 nm.
236. The compound or pharmaceutically acceptable salt thereof of any one of items 220 to 235, wherein -L²- has a Flory radius of no more than 200 nm.
237. The compound or pharmaceutically acceptable salt thereof of any one of items 220 to 236, wherein -L²- has a Flory radius of no more than 175 nm.
238. The compound or pharmaceutically acceptable salt thereof of any one of items 220 to 237, wherein -L²- has a Flory radius of no more than 150 nm.
239. The compound or pharmaceutically acceptable salt thereof of any one of items 220 to 238, wherein -L²- has a Flory radius of no more than 125 nm.
240. The compound or pharmaceutically acceptable salt thereof of any one of items 220 to 239, wherein -L²- has a Flory radius of no more than 100 nm.
241. The compound or pharmaceutically acceptable salt thereof of any one of items 220 to 240, wherein -L²- has a Flory radius of no more than 75 nm.
242. The compound or pharmaceutically acceptable salt thereof of any one of items 220 to 241, wherein -L²- has a Flory radius of no more than 50 nm.
243. The compound or pharmaceutically acceptable salt thereof of any one of items 220 to 242, wherein -L²- has a Flory radius of no more than 45 nm.
244. The compound or pharmaceutically acceptable salt thereof of any one of items 220 to 243, wherein -L²- has a Flory radius of no more than 40 nm.
245. The compound or pharmaceutically acceptable salt thereof of any one of items 220 to 244, wherein -L²- has a Flory radius of no more than 35 nm.
246. The compound or pharmaceutically acceptable salt thereof of any one of items 220 to 245, wherein -L²- has a Flory radius of no more than 30 nm.
247. The compound or pharmaceutically acceptable salt thereof of any one of items 220 to 226, wherein -L²- has a Flory radius of about 1.2 nm.
248. The compound or pharmaceutically acceptable salt thereof of any one of items 220 to 227, wherein -L²- has a Flory radius of about 1.5 nm.
249. The compound or pharmaceutically acceptable salt thereof of any one of items 220 to 227, wherein -L²- has a Flory radius of about 2 nm.
250. The compound or pharmaceutically acceptable salt thereof of any one of items 220 to 227, wherein -L²- has a Flory radius of about 2.5 nm.
251. The compound or pharmaceutically acceptable salt thereof of any one of items 220 to 227, wherein -L²- has a Flory radius of about 3 nm.
252. The compound or pharmaceutically acceptable salt thereof of any one of items 220 to 227, wherein -L²- has a Flory radius of about 3.5 nm.
253. The compound or pharmaceutically acceptable salt thereof of any one of items 220 to 227, wherein -L²- has a Flory radius of about 4 nm.
254. The compound or pharmaceutically acceptable salt thereof of any one of items 220 to 227, wherein -L²- has a Flory radius of about 4.5 nm.
255. The compound or pharmaceutically acceptable salt thereof of any one of items 220 to 227, wherein -L²- has a Flory radius of about 5 nm.
256. The compound or pharmaceutically acceptable salt thereof of any one of items 220 to 227, wherein -L²- has a Flory radius of about 5.5 nm.
257. The compound or pharmaceutically acceptable salt thereof of any one of items 220 to 227, wherein -L²- has a Flory radius of about 6 nm.
258. The compound or pharmaceutically acceptable salt thereof of any one of items 220 to 227, wherein -L²- has a Flory radius of about 6.5 nm.
259. The compound or pharmaceutically acceptable salt thereof of any one of items 220 to 227, wherein -L²- has a Flory radius of about 7 nm.
260. The compound or pharmaceutically acceptable salt thereof of any one of items 220 to 227, wherein -L²- has a Flory radius of about 7.5 nm.
261. The compound or pharmaceutically acceptable salt thereof of any one of items 220 to 227, wherein -L²- has a Flory radius of about 8 nm.
262. The compound or pharmaceutically acceptable salt thereof of any one of items 220 to 227, wherein -L²- has a Flory radius of about 8.5 nm.
263. The compound or pharmaceutically acceptable salt thereof of any one of items 220 to 227, wherein -L²- has a Flory radius of about 9 nm.
264. The compound or pharmaceutically acceptable salt thereof of any one of items 220 to 227, wherein -L²- has a Flory radius of about 9.5 nm.
265. The compound or pharmaceutically acceptable salt thereof of any one of items 220 to 227, wherein -L²- has a Flory radius of about 10 nm.
266. The compound or pharmaceutically acceptable salt thereof of any one of items 1 to 164, wherein -L²- is of formula (XII).
267. The compound or pharmaceutically acceptable salt thereof of item 266, wherein —X¹— and —X²—of formula (XII) are independently selected from the group consisting of C₁₋₂₀ alkyl, C₂₋₂₀ alkenyl, and C₂₋₂₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S, —N(R^{y3})—, —OC(OR^{y3})(R^{y3a})—, —N(R^{y3})C(O)N(R^{y3a})—, and —OC(O)N(R^{y3})—;
each T is independently selected from the group consisting of phenyl, naphthyl, indenyl, indanyl, tetralinyl, C₃₋₁₀ cycloalkyl and 3- to 10-membered heterocyclyl; wherein each T is independently optionally substituted with one or more —R^{y2}, which are the same or different; —R^{y2} is selected from the group consisting of halogen, —CN, oxo (═O), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R⁵, —R^{y5a} and —R^{y5b} is independently of each other selected from the group consisting of —H, and C₁₋₆ alkyl; wherein C₁₋₆ alkyl is optionally substituted with one or more halogen, which are the same or different.
268. The compound or pharmaceutically acceptable salt thereof of item 266 or 267, wherein -POL- of formula (XII) is of formula (XII-i).
269. The compound or pharmaceutically acceptable salt thereof of item 268, wherein y is selected such that the molecular weight of the moiety of formula (XII-i) is about 1 kDa.
270. The compound or pharmaceutically acceptable salt thereof of item 268, wherein y is selected such that the molecular weight of the moiety of formula (XII-i) is about 2 kDa.
271. The compound or pharmaceutically acceptable salt thereof of item 268, wherein y is selected such that the molecular weight of the moiety of formula (XII-i) is about 3 kDa.
272. The compound or pharmaceutically acceptable salt thereof of item 268, wherein y is selected such that the molecular weight of the moiety of formula (XII-i) is about 4 kDa.
273. The compound or pharmaceutically acceptable salt thereof of item 268, wherein y is selected such that the molecular weight of the moiety of formula (XII-i) is about 5 kDa.
274. The compound or pharmaceutically acceptable salt thereof of item 268, wherein y is selected such that the molecular weight of the moiety of formula (XII-i) is about 6 kDa.
275. The compound or pharmaceutically acceptable salt thereof of item 268, wherein y is selected such that the molecular weight of the moiety of formula (XII-i) is about 7 kDa.
276. The compound or pharmaceutically acceptable salt thereof of item 268, wherein y is selected such that the molecular weight of the moiety of formula (XII-i) is about 8 kDa.
277. The compound or pharmaceutically acceptable salt thereof of item 268, wherein y is selected such that the molecular weight of the moiety of formula (XII-i) is about 9 kDa.
278. The compound or pharmaceutically acceptable salt thereof of item 268, wherein y is selected such that the molecular weight of the moiety of formula (XII-i) is about 10 kDa.
279. The compound or pharmaceutically acceptable salt thereof of item 268, wherein y is selected such that the molecular weight of the moiety of formula (XII-i) is about 11 kDa.
280. The compound or pharmaceutically acceptable salt thereof of item 268, wherein y is selected such that the molecular weight of the moiety of formula (XII-i) is about 12 kDa.
281. The compound or pharmaceutically acceptable salt thereof of item 268, wherein y is selected such that the molecular weight of the moiety of formula (XII-i) is about 13 kDa.
282. The compound or pharmaceutically acceptable salt thereof of item 268, wherein y is selected such that the molecular weight of the moiety of formula (XII-i) is about 14 kDa.
283. The compound or pharmaceutically acceptable salt thereof of item 268, wherein y is selected such that the molecular weight of the moiety of formula (XII-i) is about 15 kDa.
284. The compound or pharmaceutically acceptable salt thereof of item 268, wherein y is selected such that the molecular weight of the moiety of formula (XII-i) is about 20 kDa.
285. The compound or pharmaceutically acceptable salt thereof of item 268, wherein y is selected such that the molecular weight of the moiety of formula (XII-i) is about 25 kDa.
286. The compound or pharmaceutically acceptable salt thereof of item 268, wherein y is selected such that the molecular weight of the moiety of formula (XII-i) is about 30 kDa.
287. The compound or pharmaceutically acceptable salt thereof of item 268, wherein y is selected such that the molecular weight of the moiety of formula (XII-i) is about 40 kDa.
288. The compound or pharmaceutically acceptable salt thereof of item 268, wherein y formula (XII-i) is 2.
289. The compound or pharmaceutically acceptable salt thereof of item 268, wherein y formula (XII-i) is about 22.
290. The compound or pharmaceutically acceptable salt thereof of item 268, wherein y formula (XII-i) is about 45.
291. The compound or pharmaceutically acceptable salt thereof of item 268, wherein y formula (XII-i) is about 68.
292. The compound or pharmaceutically acceptable salt thereof of item 268, wherein y formula (XII-i) is about 91.
293. The compound or pharmaceutically acceptable salt thereof of item 268, wherein y formula (XII-i) is about 113.
294. The compound or pharmaceutically acceptable salt thereof of item 268, wherein y formula (XII-i) is about 136.
295. The compound or pharmaceutically acceptable salt thereof of item 268, wherein y formula (XII-i) is about 160.
296. The compound or pharmaceutically acceptable salt thereof of item 268, wherein y formula (XII-i) is about 204.
297. The compound or pharmaceutically acceptable salt thereof of item 268, wherein y formula (XII-i) is about 227.
298. The compound or pharmaceutically acceptable salt thereof of item 268, wherein y formula (XII-i) is about 250.
299. The compound or pharmaceutically acceptable salt thereof of item 268, wherein y formula (XII-i) is about 340.
300. The compound or pharmaceutically acceptable salt thereof of item 268, wherein y formula (XII-i) is about 455.
301. The compound or pharmaceutically acceptable salt thereof of item 268, wherein y formula (XII-i) is about 910.
302. The compound or pharmaceutically acceptable salt thereof of any one of items 1 to 268, wherein -L²- is of formula (XII-ii).
303. The compound or pharmaceutically acceptable salt thereof of item 302, wherein a of formula (XII-ii) is 1.
304. The compound or pharmaceutically acceptable salt thereof of item 302, wherein a of formula (XII-ii) is 2.
305. The compound or pharmaceutically acceptable salt thereof of item 302, wherein a of formula (XII-ii) is 3.
306. The compound or pharmaceutically acceptable salt thereof of item 302, wherein a of formula (XII-ii) is 4.
307. The compound or pharmaceutically acceptable salt thereof of item 302, wherein a of formula (XII-ii) is 5.
308. The compound or pharmaceutically acceptable salt thereof of item 302, wherein of formula (XII-ii)a is 6.
309. The compound or pharmaceutically acceptable salt thereof of item 302, wherein a of formula (XII-ii) is 7.
310. The compound or pharmaceutically acceptable salt thereof of item 302, wherein a of formula (XII-ii) is 8.
311. The compound or pharmaceutically acceptable salt thereof of item 302, wherein a of formula (XII-ii) is 9.
312. The compound or pharmaceutically acceptable salt thereof of item 302, wherein a of formula (XII-ii) is 10.
313. The compound or pharmaceutically acceptable salt thereof of item 302, wherein a of formula (XII-ii) is 11.
314. The compound or pharmaceutically acceptable salt thereof of item 302, wherein a of formula (XII-ii) is 12.
315. The compound or pharmaceutically acceptable salt thereof of item 302, wherein a of formula (XII-ii) is 13.
316. The compound or pharmaceutically acceptable salt thereof of item 302, wherein a of formula (XII-ii) is 14.
317. The compound or pharmaceutically acceptable salt thereof of item 302, wherein a of formula (XII-ii) is 15.
318. The compound or pharmaceutically acceptable salt thereof of any one of items 302 to 317, wherein b of formula (XII-ii) is 1.
319. The compound or pharmaceutically acceptable salt thereof of any one of items 302 to 317, wherein b of formula (XII-ii) is 2.
320. The compound or pharmaceutically acceptable salt thereof of any one of items 302 to 317, wherein b of formula (XII-ii) is 3.
321. The compound or pharmaceutically acceptable salt thereof of any one of items 302 to 317, wherein b of formula (XII-ii) is 4.
322. The compound or pharmaceutically acceptable salt thereof of any one of items 302 to 317, wherein b of formula (XII-ii) is 5.
323. The compound or pharmaceutically acceptable salt thereof of any one of items 302 to 317, wherein b of formula (XII-ii) is 6.
324. The compound or pharmaceutically acceptable salt thereof of any one of items 302 to 317, wherein b of formula (XII-ii) is 7.
325. The compound or pharmaceutically acceptable salt thereof of any one of items 302 to 317, wherein b of formula (XII-ii) is 8.
326. The compound or pharmaceutically acceptable salt thereof of any one of items 302 to 317, wherein b of formula (XII-ii) is 9.
327. The compound or pharmaceutically acceptable salt thereof of any one of items 302 to 317, wherein b of formula (XII-ii) is 10.
328. The compound or pharmaceutically acceptable salt thereof of any one of items 302 to 317, wherein b of formula (XII-ii) is 11.
329. The compound or pharmaceutically acceptable salt thereof of any one of items 302 to 317, wherein b of formula (XII-ii) is 12.
330. The compound or pharmaceutically acceptable salt thereof of any one of items 302 to 317, wherein b of formula (XII-ii) is 13.
331. The compound or pharmaceutically acceptable salt thereof of any one of items 302 to 317, wherein b of formula (XII-ii) is 14.
332. The compound or pharmaceutically acceptable salt thereof of any one of items 302 to 317, wherein b of formula (XII-ii) is 15.
333. The compound or pharmaceutically acceptable salt thereof of any one of items 302 to 332, wherein c of formula (XII-ii) is 1.
334. The compound or pharmaceutically acceptable salt thereof of any one of items 302 to 332, wherein c of formula (XII-ii) is 2.
335. The compound or pharmaceutically acceptable salt thereof of any one of items 302 to 332, wherein c of formula (XII-ii) is 3.
336. The compound or pharmaceutically acceptable salt thereof of any one of items 302 to 332, wherein c of formula (XII-ii) is 4.
337. The compound or pharmaceutically acceptable salt thereof of any one of items 302 to 332, wherein c of formula (XII-ii) is 5.
338. The compound or pharmaceutically acceptable salt thereof of any one of items 302 to 332, wherein c of formula (XII-ii) is 6.
339. The compound or pharmaceutically acceptable salt thereof of any one of items 302 to 332, wherein c of formula (XII-ii) is 7.
340. The compound or pharmaceutically acceptable salt thereof of any one of items 302 to 332, wherein c of formula (XII-ii) is 8.
341. The compound or pharmaceutically acceptable salt thereof of any one of items 302 to 332, wherein c of formula (XII-ii) is 9.
342. The compound or pharmaceutically acceptable salt thereof of any one of items 302 to 332, wherein c of formula (XII-ii) is 10.
343. The compound or pharmaceutically acceptable salt thereof of any one of items 302 to 332, wherein c of formula (XII-ii) is 11.
344. The compound or pharmaceutically acceptable salt thereof of any one of items 302 to 332, wherein c of formula (XII-ii) is 12.
345. The compound or pharmaceutically acceptable salt thereof of any one of items 302 to 332, wherein c of formula (XII-ii) is 13.
346. The compound or pharmaceutically acceptable salt thereof of any one of items 302 to 332, wherein c of formula (XII-ii) is 14.
347. The compound or pharmaceutically acceptable salt thereof of any one of items 302 to 332, wherein c of formula (XII-ii) is 15.
348. The compound or pharmaceutically acceptable salt thereof of any one of items 302 to 347, wherein —R¹ of formula (XII-ii) is —H.
349. The compound or pharmaceutically acceptable salt thereof of any one of items 302 to 347, wherein —R¹ of formula (XII-ii) is C₁₋₆ alkyl.
350. The compound or pharmaceutically acceptable salt thereof of any one of items 302 to 347, wherein —R¹ of formula (XII-ii) is methyl.
351. The compound or pharmaceutically acceptable salt thereof of any one of items 302 to 349, wherein —R² of formula (XII-ii) is —H.
352. The compound or pharmaceutically acceptable salt thereof of any one of items 302 to 349, wherein —R² of formula (XII-ii) is C₁₋₆ alkyl.
353. The compound or pharmaceutically acceptable salt thereof of any one of items 302 to 349, wherein —R² of formula (XII-ii) is methyl.
354. The compound or pharmaceutically acceptable salt thereof of any one of items 302 to 353, wherein x of formula (XII-ii) is 2.
355. The compound or pharmaceutically acceptable salt thereof of any one of items 302 to 353, wherein x of formula (XII-ii) is about 22.
356. The compound or pharmaceutically acceptable salt thereof of any one of items 302 to 353, wherein x of formula (XII-ii) is about 45.
357. The compound or pharmaceutically acceptable salt thereof of any one of items 302 to 353, wherein x of formula (XII-ii) is about 68.
358. The compound or pharmaceutically acceptable salt thereof of any one of items 302 to 353, wherein x of formula (XII-ii) is about 91.
359. The compound or pharmaceutically acceptable salt thereof of any one of items 302 to 353, wherein x of formula (XII-ii) is about 136.
360. The compound or pharmaceutically acceptable salt thereof of any one of items 302 to 353, wherein x of formula (XII-ii) is about 160.
361. The compound or pharmaceutically acceptable salt thereof of any one of items 302 to 353, wherein x of formula (XII-ii) is about 182.
362. The compound or pharmaceutically acceptable salt thereof of any one of items 302 to 353, wherein x of formula (XII-ii) is about 204.
363. The compound or pharmaceutically acceptable salt thereof of any one of items 302 to 353, wherein x of formula (XII-ii) is about 227.
364. The compound or pharmaceutically acceptable salt thereof of any one of items 302 to 353, wherein x of formula (XII-ii) is about 250.
365. The compound or pharmaceutically acceptable salt thereof of any one of items 302 to 353, wherein x of formula (XII-ii) is about 340.
366. The compound or pharmaceutically acceptable salt thereof of any one of items 302 to 353, wherein x of formula (XII-ii) is about 455.
367. The compound or pharmaceutically acceptable salt thereof of any one of items 302 to 353, wherein x of formula (XII-ii) is about 910.
368. The compound or pharmaceutically acceptable salt thereof of any one of items 1 to 367, wherein -D- has the sequence of SEQ ID NO:1.
369. The compound or pharmaceutically acceptable salt thereof of any one of items 1 to 367, wherein -D- comprises one or more amino acid changes compared to SEQ ID NO: 1.
370. The compound or a pharmaceutically acceptable salt thereof of item 369, wherein -D- has a maximum of three amino acid changes compared to SEQ ID NO:1.
371. The compound or pharmaceutically acceptable salt thereof of any one of items 1 to 367, wherein -D- has the sequence of SEQ ID NO:2.
372. The compound or pharmaceutically acceptable salt thereof of any one of items 1 to 367, wherein -D- has the sequence of SEQ ID NO:3.
373. The compound or pharmaceutically acceptable salt thereof of any one of items 1 to 367, wherein -D- has the sequence of SEQ ID NO:4.
374. The compound or pharmaceutically acceptable salt thereof of any one of items 1 to 367, wherein -D- has the sequence of SEQ ID NO:5.
375. The compound or pharmaceutically acceptable salt thereof of any one of items 1 to 367, wherein -D- has the sequence of SEQ ID NO:6.
376. The compound or pharmaceutically acceptable salt thereof of any one of items 1 to 367, wherein -D- has the sequence of SEQ ID NO:7.
377. The compound or pharmaceutically acceptable salt thereof of any one of items 1 to 367, wherein -D- has the sequence of SEQ ID NO:8.
378. The compound or pharmaceutically acceptable salt thereof of any one of items 1 to 367, wherein -D- has the sequence of SEQ ID NO:9.
379. The compound or pharmaceutically acceptable salt thereof of any one of items 1 to 367, wherein -D- has the sequence of SEQ ID NO:10.
380. The compound or pharmaceutically acceptable salt thereof of any one of items 1 to 367, wherein -D- has the sequence of SEQ ID NO:11.
381. The compound or pharmaceutically acceptable salt thereof of any one of items 1 to 367, wherein -D- has the sequence of SEQ ID NO:12.
382. The compound or pharmaceutically acceptable salt thereof of any one of items 1 to 367, wherein -D- has the sequence of SEQ ID NO:13.
383. The compound or pharmaceutically acceptable salt thereof of any one of items 1 to 367, wherein -D- has the sequence of SEQ ID NO:14.
384. The compound or pharmaceutically acceptable salt thereof of any one of items 1 to 367, wherein -D- has the sequence of SEQ ID NO:15.
385. The compound or pharmaceutically acceptable salt thereof of any one of items 1 to 367, wherein -D- has the sequence of SEQ ID NO:16.
386. The compound or pharmaceutically acceptable salt thereof of any one of items 1 to 367, wherein -D- has the sequence of SEQ ID NO:17.
387. The compound or pharmaceutically acceptable salt thereof of any one of items 1 to 367, wherein -D- has the sequence of SEQ ID NO:18.
388. The compound or pharmaceutically acceptable salt thereof of any one of items 1 to 367, wherein -D- has the sequence of SEQ ID NO:19.
389. The compound or pharmaceutically acceptable salt thereof of any one of items 1 to 367, wherein -D- has the sequence of SEQ ID NO:20.
390. The compound or pharmaceutically acceptable salt thereof of any one of items 1 to 367, wherein -D- has the sequence of SEQ ID NO:21.
391. The compound or pharmaceutically acceptable salt thereof of any one of items 1 to 367, wherein -D- has the sequence of SEQ ID NO:22.
392. The compound or pharmaceutically acceptable salt thereof of any one of items 1 to 367, wherein -D- has the sequence of SEQ ID NO:23.
393. The compound or pharmaceutically acceptable salt thereof of any one of items 1 to 367, wherein -D- has the sequence of SEQ ID NO:24.
394. The compound or pharmaceutically acceptable salt thereof of any one of items 1 to 367, wherein -D- has the sequence of SEQ ID NO:25.
395. The compound or pharmaceutically acceptable salt thereof of any one of items 1 to 367, wherein -D- has the sequence of SEQ ID NO:26.
396. The compound or pharmaceutically acceptable salt thereof of any one of items 1 to 367, wherein -D- has the sequence of SEQ ID NO:27.
397. The compound or pharmaceutically acceptable salt thereof of any one of items 1 to 367, wherein -D- has the sequence of SEQ ID NO:28.
398. The compound or pharmaceutically acceptable salt thereof of any one of items 1 to 367, wherein -D- has the sequence of SEQ ID NO:29.
399. The compound or pharmaceutically acceptable salt thereof of any one of items 1 to 367, wherein -D- has the sequence of SEQ ID NO:30.
400. The compound or pharmaceutically acceptable salt thereof of any one of items 1 to 367, wherein -D- has the sequence of SEQ ID NO:31.
401. The compound or pharmaceutically acceptable salt thereof of any one of items 1 to 367, wherein -D- has the sequence of SEQ ID NO:32.
402. The compound or pharmaceutically acceptable salt thereof of any one of items 1 to 367, wherein -D- has the sequence of SEQ ID NO:33.
403. The compound or pharmaceutically acceptable salt thereof of any one of items 1 to 367, wherein -D- has the sequence of SEQ ID NO:54.
404. The compound or pharmaceutically acceptable salt thereof of any one of items 1 to 367, wherein -D- has the sequence of SEQ ID NO:55.
405. The compound or pharmaceutically acceptable salt thereof of any one of items 1 to 367, wherein -D- has the sequence of SEQ ID NO:56.
406. The compound or pharmaceutically acceptable salt thereof of any one of items 1 to 367, wherein -D- has the sequence of SEQ ID NO:57.
407. The compound or pharmaceutically acceptable salt thereof of any one of items 1 to 406, wherein -AB² is of formula (A).
408. The compound or pharmaceutically acceptable salt thereof of item 407, wherein —F⁰ of formula (A) is selected from the group consisting of formulas (a-4), (a-5), (a-6), (a-7), (a-8), (a-9), (a-10), (a-11), (a-12), (a-13), (a-14), (a-15), (a-16), (a-17), (a-18), (a-19), (a-20), (a-21), (a-22), (a-23), (a-24), (a-25), (a-26), (a-27), (a-28), (a-29), (a-30), (a-31), (a-32), (a-33), (a-34), (a-35), (a-36), (a-37), (a-38) and (a-39).
409. The compound or pharmaceutically acceptable salt thereof of item 407 or 408, wherein —F⁰ of formula (A) is of formula (a-4).
410. The compound or pharmaceutically acceptable salt thereof of item 407 or 408, wherein —F⁰ of formula (A) is of formula (a-5).
411. The compound or pharmaceutically acceptable salt thereof of item 407 or 408, wherein —F⁰ of formula (A) is of formula (a-6).
412. The compound or pharmaceutically acceptable salt thereof of item 407 or 408, wherein —F⁰ of formula (A) is of formula (a-7).
413. The compound or pharmaceutically acceptable salt thereof of item 407 or 408, wherein —F⁰ of formula (A) is of formula (a-8).
414. The compound or pharmaceutically acceptable salt thereof of item 407 or 408, wherein —F⁰ of formula (A) is of formula (a-9).
415. The compound or pharmaceutically acceptable salt thereof of item 407 or 408, wherein —F⁰ of formula (A) is of formula (a-10).
416. The compound or pharmaceutically acceptable salt thereof of item 407 or 408, wherein —F⁰ of formula (A) is of formula (a-11).
417. The compound or pharmaceutically acceptable salt thereof of item 407 or 408, wherein —F⁰ of formula (A) is of formula (a-12).
418. The compound or pharmaceutically acceptable salt thereof of item 407 or 408, wherein —F⁰ of formula (A) is of formula (a-13).
419. The compound or pharmaceutically acceptable salt thereof of item 407 or 408, wherein —F⁰ of formula (A) is of formula (a-14).
420. The compound or pharmaceutically acceptable salt thereof of item 407 or 408, wherein —F⁰ of formula (A) is of formula (a-15).
421. The compound or pharmaceutically acceptable salt thereof of item 407 or 408. wherein —F⁰ of formula (A) is of formula (a-16).
422. The compound or pharmaceutically acceptable salt thereof of item 407 or 408, wherein —F⁰ of formula (A) is of formula (a-17).
423. The compound or pharmaceutically acceptable salt thereof of item 407 or 408, wherein —F⁰ of formula (A) is of formula (a-18).
424. The compound or pharmaceutically acceptable salt thereof of item 407 or 408, wherein —F⁰ of formula (A) is of formula (a-19).
425. The compound or pharmaceutically acceptable salt thereof of item 407 or 408, wherein —F⁰ of formula (A) is of formula (a-20).
426. The compound or pharmaceutically acceptable salt thereof of item 407 or 408, wherein —F⁰ of formula (A) is of formula (a-21).
427. The compound or pharmaceutically acceptable salt thereof of item 407 or 408, wherein —F⁰ of formula (A) is of formula (a-22).
428. The compound or pharmaceutically acceptable salt thereof of item 407 or 408, wherein —F⁰ of formula (A) is of formula (a-23).
429. The compound or pharmaceutically acceptable salt thereof of item 407 or 408, wherein —F⁰ of formula (A) is of formula (a-24).
430. The compound or pharmaceutically acceptable salt thereof of item 407 or 408, wherein —F⁰ of formula (A) is of formula (a-25).
431. The compound or pharmaceutically acceptable salt thereof of item 407 or 408, wherein —F⁰ of formula (A) is of formula (a-26).
432. The compound or pharmaceutically acceptable salt thereof of item 407 or 408, wherein —F⁰ of formula (A) is of formula (a-27).
433. The compound or pharmaceutically acceptable salt thereof of item 407 or 408, wherein —F⁰ of formula (A) is of formula (a-28).
434. The compound or pharmaceutically acceptable salt thereof of item 407 or 408, wherein —F⁰ of formula (A) is of formula (a-29).
435. The compound or pharmaceutically acceptable salt thereof of item 407 or 408, wherein —F⁰ of formula (A) is of formula (a-30).
436. The compound or pharmaceutically acceptable salt thereof of item 407 or 408, wherein —F⁰ of formula (A) is of formula (a-31).
437. The compound or pharmaceutically acceptable salt thereof of item 407 or 408, wherein —F⁰ of formula (A) is of formula (a-32).
438. The compound or pharmaceutically acceptable salt thereof of item 407 or 408. wherein —F⁰ of formula (A) is of formula (a-33).
439. The compound or pharmaceutically acceptable salt thereof of item 407 or 408, wherein —F⁰ of formula (A) is of formula (a-34).
440. The compound or pharmaceutically acceptable salt thereof of item 407 or 408, wherein —F⁰ of formula (A) is of formula (a-35).
441. The compound or pharmaceutically acceptable salt thereof of item 407 or 408, wherein —F⁰ of formula (A) is of formula (a-36).
442. The compound or pharmaceutically acceptable salt thereof of item 407 or 408, wherein —F⁰ of formula (A) is of formula (a-37).
443. The compound or pharmaceutically acceptable salt thereof of item 407 or 408, wherein —F⁰ of formula (A) is of formula (a-38).
444. The compound or pharmaceutically acceptable salt thereof of item 407 or 408, wherein —F⁰ of formula (A) is of formula (a-39).
445. The compound or pharmaceutically acceptable salt thereof of any one of items 407 to 444, wherein -L^{A}- of formula (A) is absent.
446. The compound or pharmaceutically acceptable salt thereof of any one of items 407 to 444, wherein -L^{A}- of formula (A) is selected from the group consisting of formulas (a-40), (a-41), (a-42), (a-43), (a-44), (a-45), (a-46), (a-47), (a-48), (a-49), (a-50), (a-51), (a-52), (a-53), (a-54), (a-55), (a-56), (a-57), (a-58), (a-59), (a-60), (a-61), (a-62), (a-63), (a-64), (a-65), (a-66), (a-67), (a-68), (a-69), (a-70), (a-71), (a-72), (a-73), (a-74), (a-75), (a-76), (a-77), (a-78), (a-79), (a-80), (a-81) and (a-82).
447. The compound or pharmaceutically acceptable salt thereof of any one of items 407 to 444 or 446 wherein -L^{A}- of formula (A) is of formula (a-40).
448. The compound or pharmaceutically acceptable salt thereof of any one of items 407 to 444 or 446, wherein -L^{A}- of formula (A) is of formula (a-41).
449. The compound or pharmaceutically acceptable salt thereof of any one of items 407 to 444 or 446, wherein -L^{A}- of formula (A) is of formula (a-42).
450. The compound or pharmaceutically acceptable salt thereof of any one of items 407 to 444 or 446, wherein -L^{A}- of formula (A) is of formula (a-43).
451. The compound or pharmaceutically acceptable salt thereof of any one of items 407 to 444 or 446, wherein -L^{A}- of formula (A) is of formula (a-44).
452. The compound or pharmaceutically acceptable salt thereof of any one of items 407 to 444 or 446, wherein -L^{A}- of formula (A) is of formula (a-45).
453. The compound or pharmaceutically acceptable salt thereof of any one of items 407 to 444 or 446, wherein -L^{A}- of formula (A) is of formula (a-46).
454. The compound or pharmaceutically acceptable salt thereof of any one of items 407 to 444 or 446, wherein -L^{A}- of formula (A) is of formula (a-47).
455. The compound or pharmaceutically acceptable salt thereof of any one of items 407 to 444 or 446, wherein -L^{A}- of formula (A) is of formula (a-48).
456. The compound or pharmaceutically acceptable salt thereof of any one of items 407 to 444 or 446, wherein -L^{A}- of formula (A) is of formula (a-49).
457. The compound or pharmaceutically acceptable salt thereof of any one of items 407 to 444 or 446, wherein -L^{A}- of formula (A) is of formula (a-50).
458. The compound or pharmaceutically acceptable salt thereof of any one of items 407 to 444 or 446, wherein -L^{A}- of formula (A) is of formula (a-51).
459. The compound or pharmaceutically acceptable salt thereof of any one of items 407 to 444 or 446, wherein -L^{A}- of formula (A) is of formula (a-52).
460. The compound or pharmaceutically acceptable salt thereof of any one of items 407 to 444 or 446, wherein -L^{A}- of formula (A) is of formula (a-53).
461. The compound or pharmaceutically acceptable salt thereof of any one of items 407 to 444 or 446, wherein -L^{A}- of formula (A) is of formula (a-54).
462. The compound or pharmaceutically acceptable salt thereof of any one of items 407 to 444 or 446, wherein -L^{A}- of formula (A) is of formula (a-55).
463. The compound or pharmaceutically acceptable salt thereof of any one of items 407 to 444 or 446, wherein -L^{A}- of formula (A) is of formula (a-56).
464. The compound or pharmaceutically acceptable salt thereof of any one of items 407 to 444 or 446, wherein -L^{A}- of formula (A) is of formula (a-57).
465. The compound or pharmaceutically acceptable salt thereof of any one of items 407 to 444 or 446, wherein -L^{A}- of formula (A) is of formula (a-58).
466. The compound or pharmaceutically acceptable salt thereof of any one of items 407 to 444 or 446, wherein -L^{A}- of formula (A) is of formula (a-59).
467. The compound or pharmaceutically acceptable salt thereof of any one of items 407 to 444 or 446, wherein -L^{A}- of formula (A) is of formula (a-60).
468. The compound or pharmaceutically acceptable salt thereof of any one of items 407 to 444 or 446, wherein -L^{A}- of formula (A) is of formula (a-61).
469. The compound or pharmaceutically acceptable salt thereof of any one of items 407 to 444 or 446, wherein -L^{A}- of formula (A) is of formula (a-62).
470. The compound or pharmaceutically acceptable salt thereof of any one of items 407 to 444 or 446, wherein -L^{A}- of formula (A) is of formula (a-63).
471. The compound or pharmaceutically acceptable salt thereof of any one of items 407 to 444 or 446, wherein -L^{A}- of formula (A) is of formula (a-64).
472. The compound or pharmaceutically acceptable salt thereof of any one of items 407 to 444 or 446, wherein -L^{A}- of formula (A) is of formula (a-65).
473. The compound or pharmaceutically acceptable salt thereof of any one of items 407 to 444 or 446, wherein -L^{A}- of formula (A) is of formula (a-66).
474. The compound or pharmaceutically acceptable salt thereof of any one of items 407 to 444 or 446 wherein -L^{A}- of formula (A) is of formula (a-67).
475. The compound or pharmaceutically acceptable salt thereof of any one of items 407 to 444 or 446, wherein -L^{A}- of formula (A) is of formula (a-68).
476. The compound or pharmaceutically acceptable salt thereof of any one of items 407 to 444 or 446, wherein -L^{A}- of formula (A) is of formula (a-69).
477. The compound or pharmaceutically acceptable salt thereof of any one of items 407 to 444 or 446, wherein -L^{A}- of formula (A) is of formula (a-70).
478. The compound or pharmaceutically acceptable salt thereof of any one of items 407 to 444 or 446, wherein -L^{A}- of formula (A) is of formula (a-71).
479. The compound or pharmaceutically acceptable salt thereof of any one of items 407 to 444 or 446, wherein -L^{A}- of formula (A) is of formula (a-72).
480. The compound or pharmaceutically acceptable salt thereof of any one of items 407 to 444 or 446, wherein -L^{A}- of formula (A) is of formula (a-73).
481. The compound or pharmaceutically acceptable salt thereof of any one of items 407 to 444 or 446, wherein -L^{A}- of formula (A) is of formula (a-74).
482. The compound or pharmaceutically acceptable salt thereof of any one of items 407 to 444 or 446, wherein -L^{A}- of formula (A) is of formula (a-75).
483. The compound or pharmaceutically acceptable salt thereof of any one of items 407 to 444 or 446, wherein -L^{A}- of formula (A) is of formula (a-76).
484. The compound or pharmaceutically acceptable salt thereof of any one of items 407 to 444 or 446, wherein -L^{A}- of formula (A) is of formula (a-77).
485. The compound or pharmaceutically acceptable salt thereof of any one of items 407 to 444 or 446, wherein -L^{A}- of formula (A) is of formula (a-78).
486. The compound or pharmaceutically acceptable salt thereof of any one of items 407 to 444 or 446, wherein -L^{A}- of formula (A) is of formula (a-79).
487. The compound or pharmaceutically acceptable salt thereof of any one of items 407 to 444 or 446, wherein -L^{A}- of formula (A) is of formula (a-80).
488. The compound or pharmaceutically acceptable salt thereof of any one of items 407 to 444 or 446, wherein -L^{A}- of formula (A) is of formula (a-81).
489. The compound or pharmaceutically acceptable salt thereof of any one of items 407 to 444 or 446, wherein -L^{A}- of formula (A) is of formula (a-82).
490. The compound or pharmaceutically acceptable salt thereof of any one of items 407 to 489, wherein -L^{B}- of formula (A) is absent.
491. The compound or pharmaceutically acceptable salt thereof of any one of items 407 to 489, wherein -L^{B}- of formula (A) is selected from the group consisting of formulas (a-83), (a-84), (a-85), (a-86), (a-87), (a-88), (a-89), (a-90), (a-91), (a-92), (a-93) and (a-94).
492. The compound or pharmaceutically acceptable salt thereof of any one of items 407 to 489 or 491, wherein -L^{B}- of formula (A) is of formula (a-83).
493. The compound or pharmaceutically acceptable salt thereof of any one of items 407 to 489 or 491, wherein -L^{B}- of formula (A) is of formula (a-84).
494. The compound or pharmaceutically acceptable salt thereof of item 493, wherein q of formula (a-84) is an integer ranging from 3 to 45.
495. The compound or pharmaceutically acceptable salt thereof of item 493 or 494, wherein q of formula (a-84) is an integer ranging from 4 to 40.
496. The compound or pharmaceutically acceptable salt thereof of any one of items 493 to 495, wherein q of formula (a-84) is an inter ranging from 5 to 35.
497. The compound or pharmaceutically acceptable salt thereof of any one of items 493 to 496, wherein q of formula (a-84) is an inter ranging from 6 to 30.
498. The compound or pharmaceutically acceptable salt thereof of any one of items 493 to 497, wherein q of formula (a-84) is an inter ranging from 7 to 25.
499. The compound or pharmaceutically acceptable salt thereof of any one of items 493 to 498, wherein q of formula (a-84) is an inter ranging from 10 to 20.
500. The compound or pharmaceutically acceptable salt thereof of any one of items 493 to 499, wherein q of formula (a-84) is 23.
501. The compound or pharmaceutically acceptable salt thereof of any one of items 407 to 489 or 491, wherein -L^{B}- of formula (A) is of formula (a-85).
502. The compound or pharmaceutically acceptable salt thereof of any one of items 407 to 489 or 491, wherein -L^{B}- of formula (A) is of formula (a-86).
503. The compound or pharmaceutically acceptable salt thereof of any one of items 407 to 489 or 491, wherein -L^{B}- of formula (A) is of formula (a-87).
504. The compound or pharmaceutically acceptable salt thereof of any one of items 407 to 489 or 491, wherein -L^{B}- of formula (A) is of formula (a-88).
505. The compound or pharmaceutically acceptable salt thereof of any one of items 407 to 489 or 491, wherein -L^{B}- of formula (A) is of formula (a-89).
506. The compound or pharmaceutically acceptable salt thereof of any one of items 407 to 489 or 491, wherein -L^{B}- of formula (A) is of formula (a-90).
507. The compound or pharmaceutically acceptable salt thereof of any one of items 407 to 489 or 491, wherein -L^{B}- of formula (A) is of formula (a-91).
508. The compound or pharmaceutically acceptable salt thereof of any one of items 407 to 489 or 491, wherein -L^{B}- of formula (A) is of formula (a-92).
509. The compound or pharmaceutically acceptable salt thereof of any one of items 407 to 489 or 491, wherein -L^{B}- of formula (A) is of formula (a-93).
510. The compound or pharmaceutically acceptable salt thereof of any one of items 407 to 489 or 491, wherein -L^{B}- of formula (A) is of formula (a-84).
511. The compound or pharmaceutically acceptable salt thereof of any one of items 1 to 510, wherein -AB² is of formula (i).
512. The compound or pharmaceutically acceptable salt thereof of item 511, wherein n of formula (i) is 14.
513. The compound or pharmaceutically acceptable salt thereof of item 511, wherein n of formula (i) is 15.
514. The compound or pharmaceutically acceptable salt thereof of item 511, wherein n of formula (i) is 16.
515. The compound or pharmaceutically acceptable salt thereof of item 511, wherein n of formula (i) is 17.
516. The compound or pharmaceutically acceptable salt thereof of item 511, wherein n of formula (i) is 18.
517. The compound or pharmaceutically acceptable salt thereof of item 511, wherein n of formula (i) is 19.
518. The compound or pharmaceutically acceptable salt thereof of item 511, wherein n of formula (i) is 20.
519. The compound or pharmaceutically acceptable salt thereof of item 511, wherein n of formula (i) is 21.
520. The compound or pharmaceutically acceptable salt thereof of item 511, wherein n of formula (i) is 22.
521. The compound or pharmaceutically acceptable salt thereof of any one of items 511 to 520, wherein the stereocenter marked with the asterisk is in R-configuration.
522. The compound or pharmaceutically acceptable salt thereof of any one of items 511 to 520, wherein the stereocenter marked with the asterisk is in S-configuration.
523. The compound or pharmaceutically acceptable salt thereof of any one of items 1 to 406, wherein -AB² is of formula

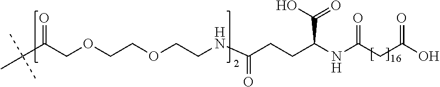

wherein the dashed line indicates attachment to -L²-.
524. The compound or pharmaceutically acceptable salt thereof of any one of items 1 to 406, wherein -AB² is of formula

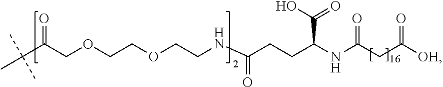

wherein the dashed line indicates attachment to -L²-.
525. The compound or pharmaceutically acceptable salt thereof of any one of item 1 to 367, wherein -D-AB² is semaglutide.
526. The compound or pharmaceutically acceptable salt thereof of any one of item 1 to 367, wherein -D-AB² is liraglutide.
527. The compound or pharmaceutically acceptable salt thereof of any one of item 1 to 367, wherein -D-AB² is ecnoglutide.
528. The compound or pharmaceutically acceptable salt thereof of any one of item 1 to 367, wherein -D-AB² is GZR18.
529. The compound or pharmaceutically acceptable salt thereof of any one of item 1 to 367, wherein -D-AB² is GL0034.
530. The compound or pharmaceutically acceptable salt thereof of any one of item 1 to 367, wherein -D-AB² is tirzepatide.
531. The compound or pharmaceutically acceptable salt thereof of any one of item 1 to 367, wherein -D-AB² is cotadutide.
532. The compound or pharmaceutically acceptable salt thereof of any one of item 1 to 367, wherein -D-AB² is BI-456906.
533. The compound or pharmaceutically acceptable salt thereof of any one of item 1 to 367, wherein -D-AB² is pemvidutide.
534. The compound or pharmaceutically acceptable salt thereof of any one of item 1 to 367, wherein -D-AB² is mazdutide.
535. The compound or pharmaceutically acceptable salt thereof of any one of item 1 to 367, wherein -D-AB² is dapiglutide.
536. The compound or pharmaceutically acceptable salt thereof of any one of item 1 to 367, wherein -D-AB² is retatrutide.
537. The compound or pharmaceutically acceptable salt thereof of any one of item 1 to 367, wherein -D-AB² is noiiglutide.
538. The compound or pharmaceutically acceptable salt thereof of any one of item 1 to 367, wherein -D-AB² is ZT002.
539. The compound or pharmaceutically acceptable salt thereof of any one of item 1 to 367, wherein -D-AB² is of formula (b-1).
540. The compound or pharmaceutically acceptable salt thereof of item 539, wherein X¹—of formula (b-1) is

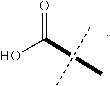

541. The compound or pharmaceutically acceptable salt thereof of item 539, wherein X¹—of formula (b-1) is

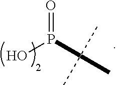

542. The compound or pharmaceutically acceptable salt thereof of item 539, wherein X¹—of formula (b-1) is

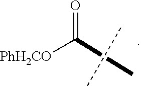

543. The compound or pharmaceutically acceptable salt thereof of item 539, wherein X¹—of formula (b-1) is

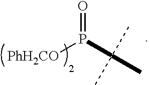

544. The compound or pharmaceutically acceptable salt thereof of any one of items 539 to 543, wherein —X² of formula (b-1) is

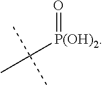

545. The compound or pharmaceutically acceptable salt thereof of any one of items 539 to 543, wherein —X² of formula (b-1) is

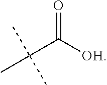

546. The compound or pharmaceutically acceptable salt thereof of any one of items 539 to 543, wherein —X² of formula (b-1) is

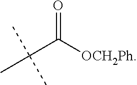

547. The compound or pharmaceutically acceptable salt thereof of any one of items 539 to 543, wherein —X² of formula (b-1) is

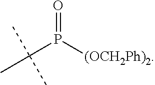

548. A pharmaceutical composition comprising at least one compound or pharmaceutically acceptable salt thereof of any one of items 1 to 547.
549. The compound or pharmaceutically acceptable salt thereof of any one of items 1 to 547 or the pharmaceutical composition of item 548 for use as a medicament.
550. The compound or pharmaceutically acceptable salt thereof or the pharmaceutical composition for use of item 549, wherein the medicament is for the treatment of a disease selected from the group consisting of (i) all forms of diabetes, (ii) obesity, (iii) non-alcoholic fatty liver disease (NAFLD) and non-alcoholic steatohepatitis (NASH), (iv) cardiovascular disease, (v) neurodegenerative disorders, (vi) chronic kidney disease (CKD), (vii) diabetic kidney disease (DKD), (viii) peripheral arterial disease (PAD), and/or (ix) heart failure (HF).
551. The compound or pharmaceutically acceptable salt thereof or the pharmaceutical composition for use of item 549, wherein the medicament is for the treatment of a disease selected from the group consisting of syndrome X, atherosclerosis, myocardial infarction, coronary heart disease, reperfusion injury, stroke, cerebral ischemia, an early cardiac or early cardiovascular disease, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (atherosclerosis obliterans), diastolic dysfunction, and/or systolic dysfunction; and reduction of blood pressure, such as reduction of systolic blood pressure.
552. The compound or pharmaceutically acceptable salt thereof or the pharmaceutical composition for use of item 549, wherein the medicament is for the treatment of a disease selected from the group consisting of dyslipidemia and/or diseases where one or more of the following clinical outcomes are the treatment goal: lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein A (apo(A)).
553. The compound or pharmaceutically acceptable salt thereof or the pharmaceutical composition for use of item 549, wherein the medicament is for the treatment of a disease selected from the group consisting of Alzheimer's disease and Parkinson's disease.
554. The compound or pharmaceutically acceptable salt thereof or the pharmaceutical composition for use of item 549, wherein the medicament is for the treatment of a disease selected from the group consisting of heart failure with reduced ejection fraction (HFrEF), heart failure with mid-range ejection fraction (HFmrEF) and heart failure with preserved ejection fraction (HFpEF).
555. The compound or pharmaceutically acceptable salt thereof or the pharmaceutical composition for use of item 549, wherein the medicament is for the treatment of a disease selected from the group consisting of non-alcoholic fatty liver disease (NAFLD) and non-alcoholic steatohepatitis (NASH).
556. The compound or pharmaceutically acceptable salt thereof or the pharmaceutical composition for use of item 549, wherein the medicament is for the treatment of a disease selected from the group consisting of is obesity and/or eating disorders, where one or more of the following clinical outcomes are the treatment goal: decreasing food intake, increasing energy expenditure, reducing body weight, suppressing appetite, inducing satiety.
557. The compound or pharmaceutically acceptable salt thereof or the pharmaceutical composition for use of any one of items 549 to 556, wherein the treatment wherein the treatment is a co-treatment with one or more additional drugs.
558. The compound or pharmaceutically acceptable salt thereof or the pharmaceutical composition for use of item 557, wherein the one or more additional drugs is selected from the group consisting of inotropes, beta adrenergic receptor blockers, HMG-CoA reductase inhibitors, angiotensin II receptor antagonists, angiotensin converting enzyme inhibitors, calcium channel blockers, endothelin antagonists, renin inhibitors, diuretics, aldosterone receptor blockers, endothelin receptor blockers, aldosterone synthase inhibitors, CETP inhibitor, relaxin, PCSK9 inhibitors, BNP and NEP inhibitors, GLP-1 analogues, insulin, sulphonylureas, biguanides, meglitinides, glucosidase inhibitors, glucagon antagonists, DPP-IV inhibitors, SGLT2 inhibitors.
559. The compound or pharmaceutically acceptable salt thereof for use of any one of items 550 to 558, wherein the treatment is combined with heart surgery.
560. The compound or pharmaceutically acceptable salt thereof or the pharmaceutical composition of any one of items 1 to 547 or the pharmaceutical composition of item 548 for use in the treatment of a disease that can be treated with a GLP-1 receptor agonist.
561. The compound or pharmaceutically acceptable salt thereof or the pharmaceutical composition for use of item 560, wherein the disease is selected from the group consisting of (i) all forms of diabetes, (ii) obesity, (iii) non-alcoholic fatty liver disease (NAFLD) and non-alcoholic steatohepatitis (NASH), (iv) cardiovascular disease, (v) neurodegenerative disorders, (vi) chronic kidney disease (CKD), (vii) diabetic kidney disease (DKD), (viii) peripheral arterial disease (PAD), and/or (ix) heart failure (HF).
562. The compound or pharmaceutically acceptable salt thereof or the pharmaceutical composition for use of item 560, wherein the disease is disease selected from the group consisting of (i) all forms of diabetes, (ii) obesity, (iii) non-alcoholic fatty liver disease (NAFLD) and non-alcoholic steatohepatitis (NASH), (iv) cardiovascular disease, (v) neurodegenerative disorders, (vi) chronic kidney disease (CKD), (vii) diabetic kidney disease (DKD), (viii) peripheral arterial disease (PAD), and/or (ix) heart failure (HF).
563. The compound or pharmaceutically acceptable salt thereof or the pharmaceutical composition for use of item 560, wherein the disease is selected from the group consisting of syndrome X, atherosclerosis, myocardial infarction, coronary heart disease, reperfusion injury, stroke, cerebral ischemia, an early cardiac or early cardiovascular disease, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (atherosclerosis obliterans), diastolic dysfunction, and/or systolic dysfunction; and reduction of blood pressure, such as reduction of systolic blood pressure.
564. The compound or pharmaceutically acceptable salt thereof or the pharmaceutical composition for use of item 560, wherein the disease is selected from the group consisting of dyslipidemia and/or diseases where one or more of the following clinical outcomes are the treatment goal: lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein A (apo(A)).
565. The compound or pharmaceutically acceptable salt thereof or the pharmaceutical composition for use of item 560, wherein the disease is selected from the group consisting of Alzheimer's disease and Parkinson's disease.
566. The compound or pharmaceutically acceptable salt thereof or the pharmaceutical composition for use of item 560, wherein the disease is selected from the group consisting of heart failure with reduced ejection fraction (HFrEF), heart failure with mid-range ejection fraction (HFmrEF) and heart failure with preserved ejection fraction (HFpEF).
567. The compound or pharmaceutically acceptable salt thereof or the pharmaceutical composition for use of item 560, wherein the disease is selected from the group consisting of non-alcoholic fatty liver disease (NAFLD) and non-alcoholic steatohepatitis (NASH).
568. The compound or pharmaceutically acceptable salt thereof or the pharmaceutical composition for use of item 560, wherein the disease is selected from the group consisting of is obesity and/or eating disorders, where one or more of the following clinical outcomes are the treatment goal: decreasing food intake, increasing energy expenditure, reducing body weight, suppressing appetite, inducing satiety.
569. The compound or pharmaceutically acceptable salt thereof or the pharmaceutical composition for use of any one of items 560 to 568, wherein the disease is a co-treatment with one or more additional drugs.
570. The compound or pharmaceutically acceptable salt thereof or the pharmaceutical composition for use of item 569, wherein the one or more additional drugs is selected from the group consisting of inotropes, beta adrenergic receptor blockers, HMG-CoA reductase inhibitors, angiotensin II receptor antagonists, angiotensin converting enzyme inhibitors, calcium channel blockers, endothelin antagonists, renin inhibitors, diuretics, aldosterone receptor blockers, endothelin receptor blockers, aldosterone synthase inhibitors, CETP inhibitor, relaxin, PCSK9 inhibitors, BNP and NEP inhibitors, GLP-1 analogues, insulin, sulphonylureas, biguanides, meglitinides, glucosidase inhibitors, glucagon antagonists, DPP-IV inhibitors, SGLT2 inhibitors.
571. The compound or pharmaceutically acceptable salt thereof for use of any one of items 560 to 568, wherein, wherein the treatment is combined with heart surgery.
572. The compound or pharmaceutically acceptable salt thereof of any one of items 1 to 547, wherein the distance between a moiety AB¹- and -AB² is such that they are capable of binding to a binding domain of two different albumins or to two different binding domains of the same albumin.
573. The compound or pharmaceutically acceptable salt thereof of any one of items 1 to 547, wherein the distance between a moiety AB¹- and -AB² is such that they are capable of binding to a binding domain of two different albumins.
574. The compound or pharmaceutically acceptable salt thereof of any one of items 1 to 547, wherein the distance between a moiety AB¹- and -AB² is such that they are capable of binding to two different binding domains of the same albumin.

### EXAMPLES

### Materials and Methods

All materials were obtained from commercial vendors except where stated otherwise. 17-((S)-1-tert-butoxycarbonyl-3-{2-[2-({2-[2-(2,5-dioxopyrrolidin-1-yloxycarbonylmeth-oxy)-ethoxy]ethylcarbamoyl}methoxy)ethoxy]ethylcarbamoyl}propylcarbamoyl)hepta-decanoic acid tert-butyl ester (alternative name: tert-butyl octadecanediol-Glu(OEG-OEG-OSu)-OtBu) is prepared as described in WO2009022005 (example 4).

Maleimide-functionalized PEG-amines MAL-PEG-NH₂, MW 1k (alternative name: Mal-PEG(1 kDa)-amine) and MAL-PEG-NH₂, MW 5k (alternative name: Mal-PEG(5 kDa)-amine) were purchased from Creative PEGWorks.

Side-chain protected [Aib8, Arg34]GLP-(7-37) on wang resin is prepared via solid phase peptide synthesis using a standard Fmoc protecting group strategy with the lysine in position 26 protected using the Mtt group as described in WO2022096636 (see General Methods for preparation of the compounds of the invention, pages 36-39). Prior to use the N-terminal Fmoc protecting group is removed by treating the resin 2×10 min with piperidine/DMF 1/4 (v/v).

### Reactions

Generally, reactions were stirred at room temperature and monitored by LCMS.

### RP-HPLC Purification

Preparative RP-HPLC purifications were performed with a Waters 600 controller with a 2487 Dual Absorbance Detector, an Agilent 1260 Infinity II preparative system, or a Knauer Azura Preparative gradient system. A Waters XBridge BEH300 Prep C18 10 μm, 150×30 mm column or XSelect CSH Prep C18 10 μm 150×30 mm column were used as stationary phase. Products were detected at 215 nm, 254 nm or 280 nm. Linear gradients of solvent system A (water containing 0.1% TFA v/v) and solvent system B (acetonitrile containing 0.1% TFA v/v) were used. HPLC fractions containing product were pooled and lyophilized if not stated otherwise.

### UPLC-MS Analysis

Analytical ultra-performance LC (UPLC)-MS was performed on a Waters Acquity system or an Agilent 1290 Infinity II equipped with a Waters BEH300 C18 column (2.1×50 mm, 1.7 μm particle size or 2.1×100 mm, 1.7 μm particle size) or with a XSelect CSH C18 (2.1×50 mm, 2.5 μm particle size); solvent A: water containing 0.04% TFA (v/v), solvent B: acetonitrile containing 0.05% TFA (v/v) coupled to a Waters Micromass ZQ, Waters SQ Detector 2 or to an Agilent Single Quad MS system.

### Example 1

### Preparation of Compound 2

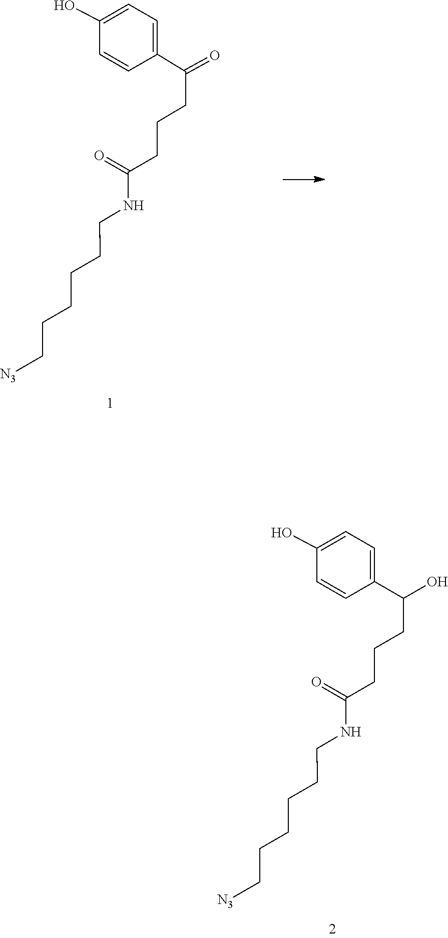 [SMILES: [N-]=[N+]=NCCCCCCNC(=O)CCCC(=O)c1ccc(O)cc1 ; [N-]=[N+]=NCCCCCCNC(=O)CCCC(O)c1ccc(O)cc1]

The acid 5-(4-hydroxyphenyl)-5-oxopentanoic acid is prepared as described in WO2009133137 (example 4) and combined with 6-azido-1-hexanamine (1 eq.) and Oxyma (1.2 eq.) in DMF. A solution of DCC (1.2 eq.) in DCM is added to the mixture, which is stirred for 3 h. The mixture is filtered, and the filtrate diluted with DCM before washing sequentially with sat. aq. NaHCO₃ then brine. The volatiles are removed in vacuo, the material is purified by normal-phase flash chromatography to give 1.

Compound 1 is dissolved in THF and under cooling in an ice-bath treated with LiBH₄ (2 eq.). After stirring for 1 h, the reaction mixture is quenched with AcOH. The solution is washed sequentially with sat. aq. NaHCO₃ and brine. After drying over Na₂SO₄ the volatiles are removed in vacuo to give 2.

### Preparation of Compound 4

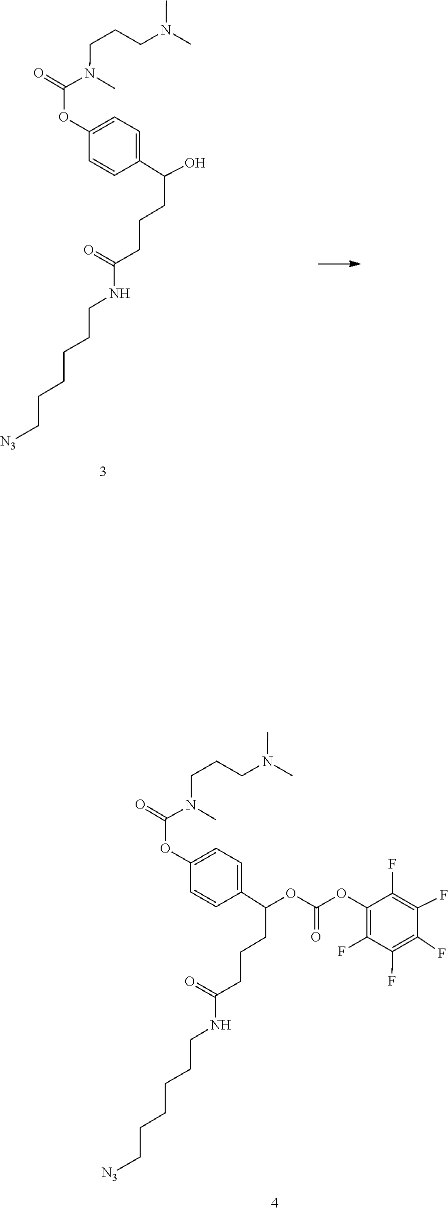 [SMILES: CN(C)CCCN(C)C(=O)Oc1ccc(C(O)CCCC(=O)NCCCCCCN=[N+]=[N-])cc1 ; CN(C)CCCN(C)C(=O)Oc1ccc(C(CCCC(=O)NCCCCCCN=[N+]=[N-])OC(=O)Oc2c(F)c(F)c(F)c(F)c2F)cc1]

To the phenol 2 in DCM is added DIPEA (2.0 eq.) and 4-nitrophenyl chloroformate (1.5 eq.). After stirring for 45 mins, N,N,N′-trimethyl-1,3-propanediamine (1.25 eq.) is added and the mixture is left to stir for 1 h. The reaction is quenched with TFA, the solvent is removed in vacuo, and the crude material is purified by prep-HPLC to give 3.

The alcohol 3 is dissolved in acetonitrile and the solution cooled in an ice-bath. DIPEA (5.0 eq.), bis(pentafluorophenyl) carbonate (2.0 eq.), and DMAP (0.1 eq.) are then added to the solution. After 90 mins, the reaction is quenched with TFA, the solvent is removed in vacuo, and the crude material is purified by prep-HPLC to give 4.

### Example 2

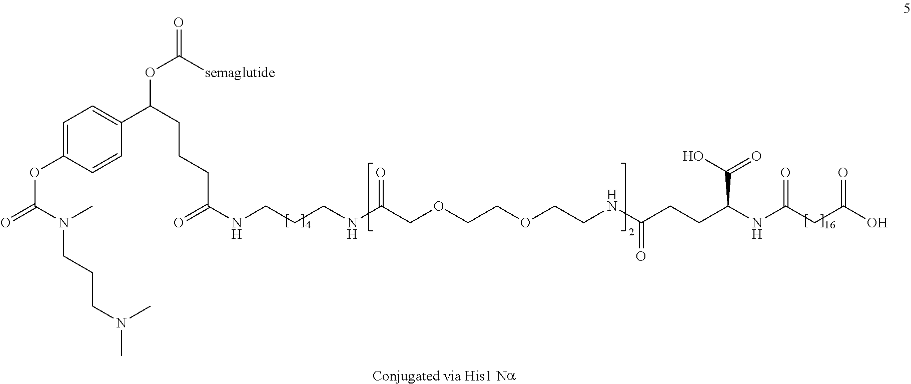

Side-chain protected [Aib8, Arg34]GLP-(7-37) on wang resin is suspended with 4 (1.5 eq.), and DIPEA (10 eq.) in DMF. The mixture is shaken at rt for 2 h then washed with DMF (5×). The azide group is reduced with TCEP (3.0 eq.) in DMF for 2 h, and the resin is shaken with 1:3 water:DMF for 1 h. The primary amine is then functionalized with tert-butyl octadecanediol-Glu(OEG-OEG-OSu)-OtBu using standard solid phase peptide coupling chemistry. The Mtt protecting group on Lysine at position 26 is selectively removed by treatment with HFIP/TIPS/DCM (75:2.5:22.5) (2×20 min), the resin is washed with DCM then DMF, then treated with tert-butyl octadecanediol-Glu(OEG-OEG-OSu)-OtBu (2.0 eq.) and DIPEA (5 eq.) in DMF. After shaking for 2 h, the peptide is cleaved with TFA/TIPS/water/DTT (95:2:2:1) for 2 h. The cleavage solution is added to ice-cold diethyl ether and centrifuged. The precipitate is collected and purified by prep-HPLC to give 5.

### Example 3

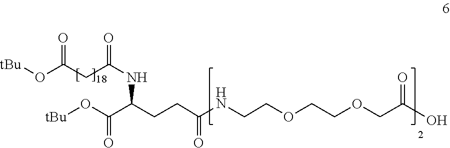

Tert-butyl protected C20-diacid-γGlu-OEG-OEG-COOH 6 was prepared according to the procedure described in WO2020159949 (see preparation 6 synthesis by method 2).

### Example 4

### Preparation of Compound 7

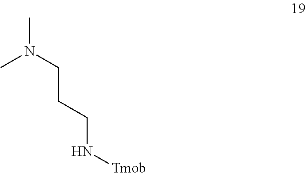

### Compound 19:

19 was prepared following the procedure described in WO2020064847 (example 11).

MS: m/z, 283.15=[M+H]⁺, (calculated [M+H]⁺=283.20).

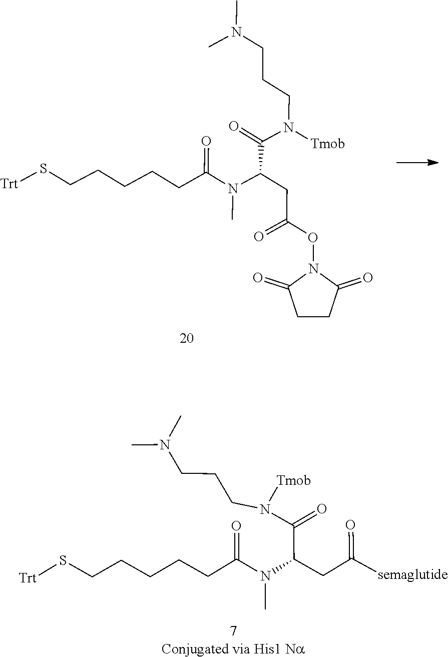

### Compound 20:

20 was prepared as the TFA salt from 19 according to the procedure described in WO2020064847 (example 11).

MS: m/z, 881.36=[M+H]⁺, (calculated [M+H]⁺=881.42).

### Compound 7:

Semaglutide (5 mg, 1.0 eq., 1.22 μmol) was dissolved in a mixture of 100 mM pH 8 borate buffer (0.5 mL) and acetonitrile (0.5 mL). To this was added 20 (7.3 mg, 6.0 eq., 7.29 μmol) dissolved in acetonitrile (73 μL). After stirring at rt for 21 h, the reaction was quenched by addition of 10% aq. TFA (20 μL) and purified directly by preparative RP-HPLC to give 7 as the TFA salt.

Yield: 1.6 mg (25%, TFA salt)

MS: m/z 1627.74=[M+3H]³⁺, (calculated [M+3H]³⁺=1627.17).

### Example 5

### Preparation of Compound 9

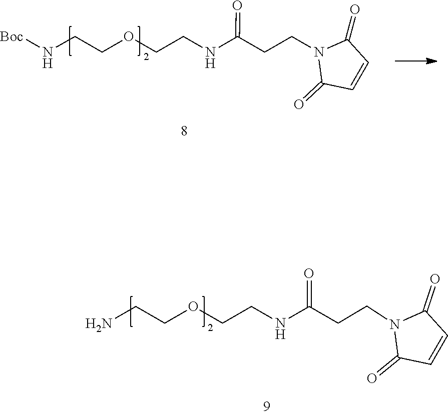

### Compound 8:

To a mixture of 3-(Maleimido)propionic acid NHS ester (59.0 mg, 1.1 eq, 221 μmol) and N-Boc-2,2′-(ethylenedioxy)diethylamine (50.0 mg, 1.0 eq, 201 μmol) in acetonitrile (1.0 mL) was added DIPEA (28.6 mg, 38.6 μL, 1.1 Eq, 221 μmol). After stirring at rt for 35 min the reaction was quenched by addition of TFA (30 μL) and water (1 mL). The product was purified by preparative RP-HPLC to give 8.

Yield 76.4 mg (95%)

MS: m/z 400.43=[M+H]⁺, (calculated [M+H]⁺=400.45)

### Compound 9:

To a solution of 8 (153.1 mg, 1.0 eq, 0.383 mmol) in DCM (2.0 mL) was added TFA (1.0 mL). After stirring at rt for 2 h the reaction was diluted with DCM (4.0 mL) and the solvent was removed under reduced pressure. The crude was mixed with 1:1 acetonitrile/water (4.0 mL) and freeze-dried to give 9 as TFA salt.

Yield 164 mg (103%, TFA salt)

MS: m/z 300.32=[M+H]⁺, (calculated [M+H]⁺=300.33)

### Example 6

### Preparation of Compounds 13-15

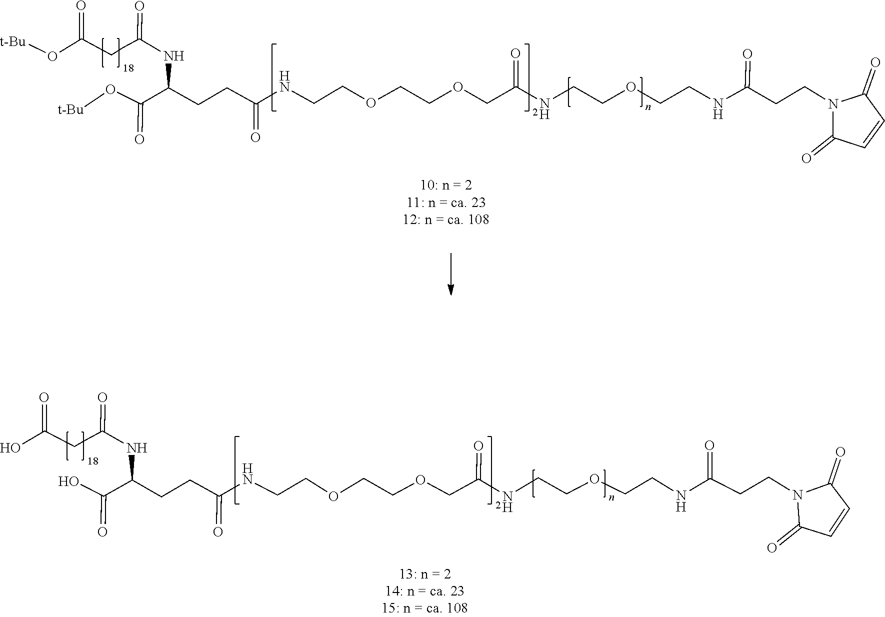

### Compounds 10 and 11:

To a solution of the fatty acid 6 in acetonitrile was added DIC and DMAP. After 5 min the respective Mal-PEG-amine (1.0 eq) was added as solution in acetonitrile. The reaction solution was shaken at rt and monitored by LCMS. Upon completion the product was purified by preparative RP-HPLC.

Synthesis of 10: 9 (6.5 mg), 6 (2.8 eq), DIC (4.1 eq), DMAP (0.5 eq): yield: 5.7 mg (31%)

MS: m/z 1178.09=[M+Na]⁺, (calculated [M+Na]⁺=1178.47)

Synthesis of 11: Mal-PEG(1 kDa)-amine (21.5 mg), 6 (1.2 eq), DIC (2.0 eq), DMAP (0.3 eq): yield: 11.3 mg (31%)

### Compound 12:

To a solution of the fatty acid 6 (79.5 mg, 0.091 mmol, 3.0 eq) in acetonitrile (1.0 mL) was added TSTU (27.4 mg, 0.091 mmol, 3.0 eq) and DIPEA (21.1 μL, 15.7 mg, 0.121 mmol, 4.0 eq). After 70 min Mal-PEG(5 kDa)-amine (150 mg, 0.030 mmol, 1.0 eq) and DIPEA (15.8 μL, 11.8 mg, 0.091 mmol, 3.0 eq) were added. After a further 70 min the reaction was quenched by addition of TFA (16.4 μL) and purified by preparative HPLC to give 12.

Yield: 99.5 mg (57%)

### Compounds 13-15:

The respective starting material was dissolved in DCM and the same volume of TFA was added. The reaction solution was shaken at rt and monitored by LCMS. Upon completion the solvent was removed under reduces pressure and the product was purified by preparative RP-HPLC.

Synthesis of 13: 10 (5.7 mg): yield: 3.9 mg (76%, TFA salt)

MS: m/z 1044.02=[M+H]⁺, (calculated [M+H]⁺=1044.26)

Synthesis of 14: 11 (11.3 mg): yield: 7.1 mg (66%, TFA salt)

Synthesis of 15: 12 (99.5 mg): yield: 70.1 mg (72%, TFA salt)

### Example 7

### Preparation of Compounds 16-18

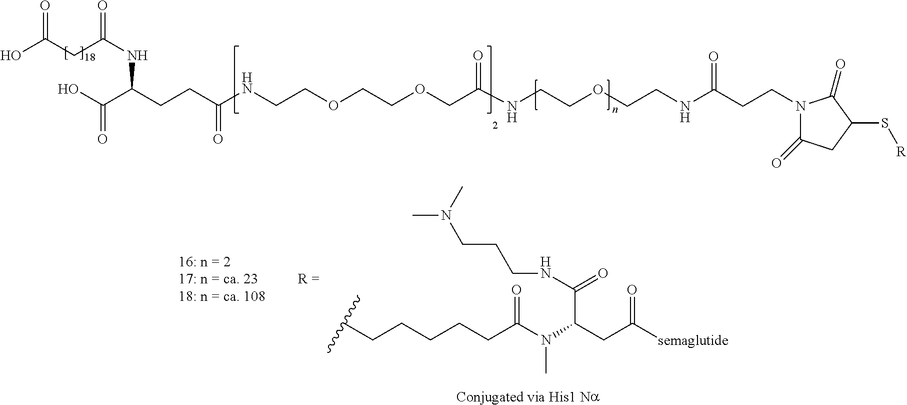

An excess of the respective maleimide in acetonitrile/water was added to a solution of 7 (1.0 eq) in acetonitrile/water. The pH of the reaction was adjusted by addition of pH 7.4 sodium phosphate buffer. The reaction solution was shaken at rt and monitored by LCMS. Upon completion the reaction was acidified with TFA. The product was purified by preparative RP-HPLC.

Synthesis of 16: 1.5 eq 13 (3.9 mg): yield: 13.3 mg (91%, TFA salt)

MS: m/z 1376.63=[M+4H]⁴⁺, (calculated [M+4H]⁴⁺=1376.10)

Synthesis of 17: 1.2 eq 14 (5.8 mg): yield: 15.8 mg (94%, TFA salt)

Synthesis of 18: 1.5 eq 15 (22.1 mg): yield: 21.8 mg (80%, TFA salt)

### Example 8: PK Study in Sprague Dawley Rats

The objective of the study was to determine the PK characteristics of 16, 17 and 18 when administered once by the IV route to the adult male Sprague Dawley rat.

The Study design is shown in Table 1. Blood samples were processed to plasma and provided for PK analysis of released semaglutide.

TABLE 1
Design of Study

| | | | | Blood sampling |
|---|---|---|---|---|
| | Test | | Dose | timepoints |
| Group | item | n | (mg/kg) | (hours post dose) |
| 1 | 16 | 3 | 0.45 | 0.25, 3, 8, 24, 36, 48, 60, |
| | | | | 72, 120, 168 and 264 |
| 2 | 17 | 3 | 0.45 | 0.25, 3, 8, 24, 36, 48, 60, |
| | | | | 72, 120, 168 and 264 |
| 3 | 18 | 3 | 0.45 | 0.25, 3, 8, 24, 36, 48, 60, |
| | | | | 72, 120, 168 and 264 |

(Dose is in mg semaglutide equivalents/kg)

### Example 9: PK Analysis of Semaglutide Released from Compounds 16, 17 and 18 in Sprague Dawley Rats

The PK of semaglutide released from 16, 17 and 18 was determined after IV administration of 16, 17 and 18 to rats according to the study described in example 8.

Blood samples were collected from all animals according to Table 1. Plasma samples were analyzed for semaglutide.

Semaglutide released from 16, 17 and 18 was quantified in Sprague Dawley rat K₃EDTA plasma via LC-MS/MS after plasma protein precipitation. Samples containing semaglutide were pipetted into the wells of a 96-well plate, followed by the addition of internal standard (d₈-semaglutide). Plasma protein precipitation was carried out using a mixture of acetonitrile and water (85/15, v/v). The precipitate was centrifuged at 4° C. and a fraction of the supernatant was transferred to a new 96-well plate, evaporated to dryness under a stream of heated nitrogen and reconstituted in water containing 0.2% formic acid.

Chromatography was performed on a Waters Acquity UPLC Peptide BEH C₁₈ analytical column (1.7 μm particle size; pore size 300 Å; column dimensions 50×2.1 mm). Water (UPLC grade) containing 0.1% formic acid (v/v) was used as mobile phase A and acetonitrile (UPLC grade) with 0.1% formic acid as mobile phase B. Multiple reaction monitoring (MRM) was performed for semaglutide and the internal standard and quantification was based on peak area and linear regression with 1/x² weighting.

A summary of the mean PK parameters for semaglutide released from 16, 17 and 18 in rat plasma are shown below in Table 2.

TABLE 2
Summary of the Mean PK Parameters of semaglutide released from
16, 17 and 18 in rat plasma in study described in example 8.

| | | | Dose | | | | | |
|---|---|---|---|---|---|---|---|---|
| Dose | Test | | level | Cₘₐₓ | tₘₐₓ | tₗₐₛₜ | AUCₗₐₛₜ | t_{1/2} |
| group | Item | ROA | (μg/kg) | (ng/mL)^{a} | (h)^{b} | (h)^{b} | (h*ng/mL)^{a} | (h)^{a} |
| 1 | 16 | IV | 450 | 49 ± 1.5 | 24 | 120 | 3,200 ± 380 | 31 ± 3.0 |
| 2 | 17 | IV | 450 | 54 ± 3.1 | 24 | 120 | 3,500 ± 82 | 33 ± 1.6 |
| 3 | 18 | IV | 450 | 72 ± 6.8 | 24 | 168 | 5,400 ± 310 | 41 ± 1.1 |

^{a}mean value ± SD;
^{b}median value

### ABBREVIATIONS

Ac acetyl
Aib 2-aminoisobutyric acid
aq aqueous
Boc tert-butyloxycarbonyl
DCC N,N′-dicyclohexylcarbodiimide
DCM dichloromethane
DIC N,N′-diisopropylcarbodiimide
DIPEA diisopropylethylamine
DMAP 4-dimethylaminopyridine
DMF dimethylformamide
DTT dithiothreitol
eq equivalent
FA fatty acid
h hour(s)
HPLC high performance liquid chromatography
HFIP hexafluoroisopropanol
LCMS liquid chromatography mass spectrometry
Mal maleimide
MS mass spectrometry
Mtt 4-methyltrityl
NHS N-hydroxysuccinimide
Oxyma cyano-hydroxyimino-acetic acid ethyl ester
PEG polyethylene glycol
prep-HPLC preparative high performance liquid chromatography
ROA route of administration
RP reversed phase
rt room temperature
sat saturated
TCEP tris(2-carboxyethyl)phosphine
TFA trifluoroacetic acid
THF tetrahydrofuran
TIPS triisopropylsilane
TSTU O—[N-Succinimidyl)-1,1,3,3-tetramethyluronium tetrafluoroborate
UPLC-MS ultra performance liquid chromatography coupled to mass spectrometry

1-26. (canceled)

27. A compound of formula (XIII) (XIII); 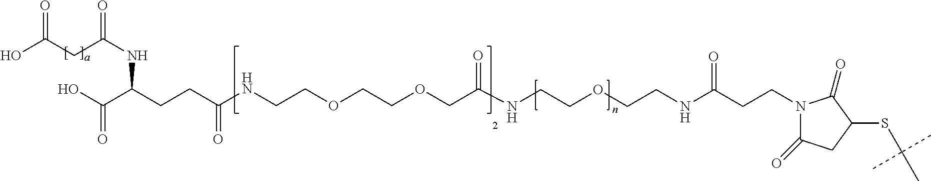
wherein
a is 18;
n is about 108;
the dashed line indicates attachment to a moiety 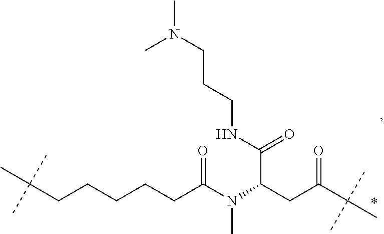
wherein
the unmarked dashed line indicates attachment to the dashed line in formula (XIII);
the dashed line marked with the asterisk indicates attachment to the N-terminal amine functional group of semaglutide; and
“about” indicates variation of ±10%.

28. A method of treating or controlling in a patient one or more diseases that can be treated with semaglutide, the method comprising the step of administering to the patient a therapeutically effective amount of the compound of claim 26.

29. The method of claim 28, wherein the disease is selected from the group consisting of (i) all forms of diabetes, (ii) obesity, (iii) non-alcoholic fatty liver disease (NAFLD) and non-alcoholic steatohepatitis (NASH), (iv) cardiovascular disease, (v) neurodegenerative disorders, (vi) chronic kidney disease (CKD), (vii) diabetic kidney disease (DKD), (viii) peripheral arterial disease (PAD), and/or (ix) heart failure (HF).

30. The method of claim 29, wherein the compound is administered once a month.

31. The method of claim 30, wherein the administration is via subcutaneous administration.

32. The method of claim 28, wherein the disease is selected from the group consisting of obesity and eating disorders, where one or more of the following clinical outcomes are the treatment goal: decreasing food intake, increasing energy expenditure, reducing body weight, suppressing appetite, inducing satiety.

33. The method of claim 32, wherein the compound is administered once a month.

34. The method of claim 33, wherein the administration is via subcutaneous administration.

35. The method of claim 28, wherein the disease is diabetes.

36. The method of claim 35, wherein the compound is administered once a month.

37. The method of claim 36, wherein the administration is via subcutaneous administration.

38. A pharmaceutical composition comprising the compound of claim 26 and at least one excipient.

39. A method of treating or controlling in a patient one or more diseases that can be treated with semaglutide, the method comprising the step of administering to the patient a therapeutically effective amount of the pharmaceutical composition of claim 38.

40. The method of claim 39, wherein the disease is selected from the group consisting of (i) all forms of diabetes, (ii) obesity, (iii) non-alcoholic fatty liver disease (NAFLD) and non-alcoholic steatohepatitis (NASH), (iv) cardiovascular disease, (v) neurodegenerative disorders, (vi) chronic kidney disease (CKD), (vii) diabetic kidney disease (DKD), (viii) peripheral arterial disease (PAD), and/or (ix) heart failure (HF).

41. The method of claim 40, wherein the pharmaceutical composition is administered once a month.

42. The method of claim 41, wherein the administration is via subcutaneous administration.

43. The method of claim 39, wherein the disease is selected from the group consisting of obesity and eating disorders, where one or more of the following clinical outcomes are the treatment goal: decreasing food intake, increasing energy expenditure, reducing body weight, suppressing appetite, inducing satiety.

44. The method of claim 43, wherein the pharmaceutical composition is administered once a month.

45. The method of claim 44, wherein the administration is via subcutaneous administration.

46. The method of claim 39, wherein the disease is diabetes.

47. The method of claim 47, wherein the pharmaceutical composition is administered once a month.

48. The method of claim 48, wherein the administration is via subcutaneous administration.
